SEQUENCE LISTING (18 sequences)

SEQ ID NO: 1 - protein, 5 aa, Artificial Sequence
  KIT protein sequence
EVQWK

SEQ ID NO: 2 - protein, 18 aa, Artificial Sequence
  KIT protein sequence
EVQWKVVEEINGNNYVYI

SEQ ID NO: 3 - protein, 9 aa, Artificial Sequence
  KIT protein sequence
KPMYEVQWK

SEQ ID NO: 4 - protein, 8 aa, Artificial Sequence
  KIT protein sequence
KPMYEVQW

SEQ ID NO: 5 - protein, 6 aa, Artificial Sequence
  KIT protein sequence
MYEVQW

SEQ ID NO: 6 - protein, 4 aa, Artificial Sequence
  KIT protein sequence
PMYE

SEQ ID NO: 7 - protein, 5 aa, Artificial Sequence
  KIT protein sequence
WKVVE

SEQ ID NO: 8 - protein, 4 aa, Artificial Sequence
  KIT protein sequence
WKVV

SEQ ID NO: 9 - protein, 6 aa, Artificial Sequence
  KIT protein sequence
YEVQWK

SEQ ID NO: 10 - protein, 5 aa, Artificial Sequence
  PDGFRA protein sequence
RVIES

SEQ ID NO: 11 - protein, 6 aa, Artificial Sequence
  PDGFRA protein sequence
SPDGHE

SEQ ID NO: 12 - protein, 5 aa, Artificial Sequence
  PDGFRA protein sequence
YDSRW

SEQ ID NO: 13 - protein, 4 aa, Artificial Sequence
  PDGFRA protein sequence
DIMH

SEQ ID NO: 14 - protein, 4 aa, Artificial Sequence
  PDGFRA protein sequence
IMHD

SEQ ID NO: 15 - protein, 4 aa, Artificial Sequence
  PDGFRA protein sequence
MHDS

SEQ ID NO: 16 - protein, 5 aa, Artificial Sequence
  PDGFRA protein sequence
DIMHD

SEQ ID NO: 17 - protein, 5 aa, Artificial Sequence
  PGDFRA protein sequence
IMHDS

SEQ ID NO: 18 - protein, 4 aa, Artificial Sequence
  PDGFRA protein sequence
HDSN

(19) United States
(12) Patent Application Publication
(10) Pub. No.: US 2024/0254130 A1
(43) Pub. Date: Aug. 1, 2024

(54) SALT AND SOLID FORMS OF A KINASE INHIBITOR

(71) Applicant: Blueprint Medicines Corporation, Cambridge, MA (US)

(72) Inventors: Erika Butler, Halifx (CA); Nimita Dave, Cambridge, MA (US); Tuan Dong Si, Cambridge, MA (US); Brian Heinrich, Cambridge, MA (US); Caitlin N. Kinkema, Cambridge, MA (US); Christopher Lee, Cambridge, MA (US); Hui Li, Cambridge, MA (US); Lauren MacEachern, Dartmouth (CA); Clare Aubrey Medendorp, Cambridge, MA (US)

(21) Appl. No.: 18/549,769

(22) PCT Filed: Mar. 10, 2022
(86) PCT No.: PCT/US2022/019776

§ 371 Date: Sep. 8, 2023

Related U.S. Application Data
(60) Provisional application No. 63/208,641, filed on Jun. 9, 2021.
(60) Provisional application No. 63/159,107, filed on Mar. 10, 2021.

(51) Int. Cl. C07D 487/04; A61K 31/53; A61P 35/00

(52) U.S. Cl. CPC C07D 487/04; A61K 31/53; A61P 35/00

(57) ABSTRACT

Various salt forms and free base solid forms of Compound (I) represented by the following formula are disclosed. Pharmaceutical compositions comprising the same, methods of treating disorders and conditions associated with oncogenic KIT and PDGFRA alterations using the same, and methods for making the salt forms of Compound (I) and crystalline forms thereof are also disclosed.

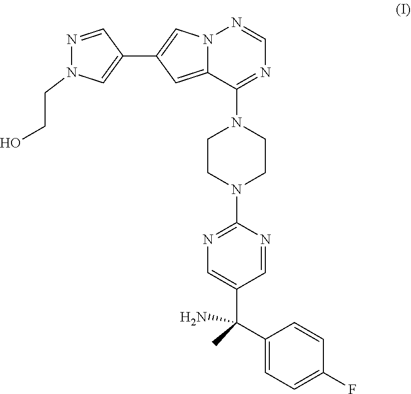 [SMILES: C[C@](N)(c1ccc(F)cc1)c1cnc(N2CCN(c3ncnn4cc(-c5cnn(CCO)c5)cc34)CC2)nc1]

### CROSS-REFERENCE TO RELATED APPLICATIONS

This application claims priority to U.S. Provisional Application No. 63/159,107, filed Mar. 10, 2021 and U.S. Provisional Application No. 63/208,641, filed Jun. 9, 2021. The entire contents of the aforementioned applications are incorporated herein by reference.

### BACKGROUND

The enzyme KIT (also called CD117) is a receptor tyrosine kinase expressed on a wide variety of cell types. The KIT receptor protein belongs to the class III receptor tyrosine kinase (RTK) family that also includes the structurally related proteins PDGFRα (platelet-derived growth factor receptor A), PDGFRβ, FLT3 (FMS-like tyrosine kinase 3), and CSF1R (colony-stimulating factor 1 receptor). The KIT molecule contains a long extracellular domain, a transmembrane segment, and an intracellular portion. The ligand for KIT is stem cell factor (SCF). Normally, stem cell factor (SCF) binds to and activates KIT by inducing dimerization, autophosphorylation, and initiation of downstream signaling. In several tumor types, however, somatic activating mutations in KIT drive ligand-independent constitutive activity.

KIT mutations generally occur in the DNA encoding the juxtumembrane domain (exon 11). KIT mutations also occur, with less frequency, in exons 7, 8, 9, 13, 14, 17, and 18. Mutations make KIT function independent of activation by SCF, leading to a high cell division rate and possibly genomic instability. Mutant KIT has been implicated in the pathogenesis of several disorders and conditions, e.g., mastocytosis, gastrointestinal stromal tumors (GIST), acute myeloid leukemia (AML), melanoma, and seminoma.

The structurally related platelet-derived growth factor receptors (PDGFR) are cell surface tyrosine kinase receptors for members of the platelet-derived growth factor (PDGF) family. PDGF subunits-α and -β regulate cell proliferation, cellular differentiation, cell growth, and cellular development. Alterations in PDGF subunit-α and -β (e.g., mutations) are associated with many diseases, including some cancers. For example, an exon 18 PDGFRα D842V mutation has been found in a distinct subset of GIST, typically from the stomach. The D842V mutation is also associated with tyrosine kinase inhibitor resistance. In addition, other exon 18 mutations such as PDGFRα D842I and PDGFRα D842Y are associated with ligand-independent, constitutive activation of PDGFRα. In GIST, gain of function mutations (such as, e.g., PDGFRα D842I, D842V, and D842Y) that confer ligand-independent constitutive activation of PDGFRα signaling have been identified as drivers of disease.

U.S. Pat. No. 10,829,493, the entire teachings of which are incorporated herein by reference, discloses potent, highly selective inhibitors of KIT, including exon 17 mutant and/or PDGFRα exon 18 mutant proteins. The structure of one of the inhibitors disclosed in U.S. Pat. No. 10,829,493, referred to herein as “Compound (I)” is shown below:

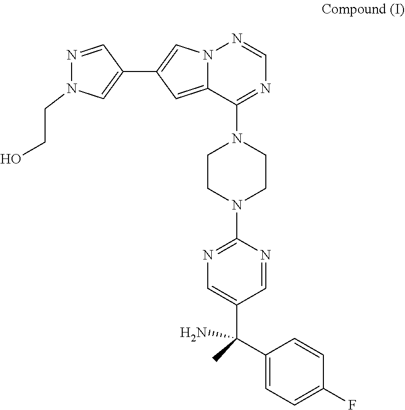 [SMILES: C[C@](N)(c1ccc(F)cc1)c1cnc(N2CCN(c3ncnn4cc(-c5cnn(CCO)c5)cc34)CC2)nc1]

Compound (I) is a potent, selective, small molecule inhibitor of the KIT exon 17 mutant enzyme, KIT D816V. Its potency is demonstrated in vitro in both the biochemical (dissociation constant, Kd=0.24 nM) and cellular (half-maximal inhibitory concentration, [IC50]=4.3 nM) settings. Compound (I) has a high degree of selectivity for KIT D816V when compared against other kinases, transmembrane or soluble receptors, ion channels, transporters, and other enzymes. Compound (I) has limited brain penetration potential.

The successful development of pharmaceutically active agents, such as Compound (I), typically requires the identification of a solid form with properties that enable ready isolation and purification following synthesis, that are amendable to large scale manufacture, that can be stored for extended periods of time with minimal absorption of water, decomposition or transformation into other solid forms, that are suitable for formulation and that can be readily absorbed following administration to the subject (e.g., are soluble in water and in gastric fluids).

### SUMMARY

It has now been found that the free base of Compound (I) has multiple crystalline forms which may interconvert with each other because of different amounts of water residing in the crystal lattice. As a consequence, preparation of a stable crystalline form of the free base of Compound (I) was found to be difficult to reproduce on a production scale.

It has now also been found that the 1:1 phosphate salt, the 2:1 besylate salt, the 1:1 benzoate salt, and the 1:1 sulfate salt can be crystallized under well-defined conditions to provide non-hygroscopic crystalline forms. These four salts demonstrated good thermal behavior with high melting point onsets and are suitable for large scale synthesis. Minimal mass loss was observed during thermogravimetric analysis. The salts disclosed herein also demonstrated similar solubility in simulated fluids and water at 37° C.

In addition, the 1:1 phosphate salt maintained a purity greater than 98%. Among the crystalline forms of the 1:1 phosphate salt, it was found that anhydrous crystalline form A of the phosphate salt is the most stable form. Form A was highly crystalline with a melting onset of 199.7° C. Form A demonstrated low residual solvent (0.45 wt. %), and good solubility in simulated fluids and water. Form A also exhibited low solubility in the organic solvents and solvent mixtures selected, but was soluble in solvents with high boiling points, such as dimethylacetamide (DMAc), n-methyl pyrrolidone (NMP), and dimethyl sulfoxide (DMSO), and in aqueous/organic mixtures, particularly with tetrahydrofuran (THF). Form A was obtained from different crystallization experiments and in slurries with alcohols, acetates, acetone, methyl ethyl ketone (MEK), trifluoroethanol (TFE), dioxane, chlorobenzene, chloroform, anisole and in many aqueous/organic mixtures. Crystallization using 2-MeTHF was advantageous in that it provided crystalline Compound (I) phosphate salt Form A, with a robust control of residual solvent.

The designation “1:1” is the molar ratio between Compound (I) and acid (e.g., sulfuric acid, phosphoric acid or benzoic acid); and the designation “2:1” is the molar ratio between Compound (I) and acid (e.g., benzene sulfonic acid).

In one aspect, the present disclosure provides a phosphate salt of Compound (I) wherein the molar ratio between Compound (I) and phosphoric acid is 1:1.

In another aspect, the present disclosure provides a besylate salt of Compound (I) wherein the molar ratio between Compound (I) and benzene sulfonic acid is 2:1.

In yet another aspect, the present disclosure provides a sulfate salt of Compound (I) wherein the molar ratio between Compound (I) and sulfuric acid is 1:1.

In yet another aspect, the present disclosure provides a benzoate salt of Compound (I) wherein the molar ratio between Compound (I) and benzoic acid is 1:1.

In another aspect, the present disclosure provides a pharmaceutical composition comprising the salt or the free base (including both amorphous form and crystalline form) of Compound (I) disclosed herein and a pharmaceutically acceptable carrier or diluent.

The present disclosure provides a method of treating disorders and conditions associated with oncogenic KIT and PDGFRA alterations, comprising administering to a patient in need thereof the salt or the free base (including both amorphous form and crystalline form) of Compound (I) disclosed herein, or the corresponding pharmaceutical composition thereof.

The present disclosure provides a method of treating a disease or condition in a patient in need thereof, comprising administering to a patient in need thereof the salt or the free base (including both amorphous form and crystalline form) of Compound (I) disclosed herein, or the corresponding pharmaceutical composition thereof, wherein the disease or condition is chosen from systemic mastocytosis, gastrointestinal stromal tumors, acute myeloid leukemia, melanoma, seminoma, intercranial germ cell tumors, mediastinal B-cell lymphoma, Ewing's sarcoma, diffuse large B cell lymphoma, dysgerminoma, myelodysplastic syndrome, nasal NK/T-cell lymphoma, chronic myelomonocytic leukemia, and brain cancer. In one embodiment, the disease or condition is chosen from systemic mastocytosis, gastrointestinal stromal tumors, acute myeloid leukemia, melanoma, seminoma, mediastinal B-cell lymphoma, Ewing's sarcoma, diffuse large B cell lymphoma, dysgerminoma, myelodysplastic syndrome, nasal NK/T-cell lymphoma, and chronic myelomonocytic leukemia. In one embodiment, the disease or condition is systemic mastocytosis. In one embodiment, the systemic mastocytosis is chosen from indolent systemic mastocytosis and smoldering systemic mastocytosis.

The present disclosure also provides a use of the salt or freebase of Compound (I) of the disclosure or a pharmaceutical composition thereof comprising the same for the treatment of any of the disease recited in the previous paragraph. In one embodiment, provided is the salt or freebase of the disclosure or a pharmaceutical composition thereof comprising the same for use in any of the method of the disclosure described herein. In another embodiment, provided is use of the salt of the disclosure or freebase or a pharmaceutical composition thereof comprising the same for the manufacture of a medicament for any of the method of the disclosure described.

### BRIEF DESCRIPTION OF THE DRAWINGS

FIG. 1A shows the X-ray Powder Diffraction (XRPD) pattern of Form A of phosphate salt of Compound (I), wherein the molar ratio between Compound (I) and phosphoric acid is 1:1.

FIG. 1B shows the Thermogravimetric Analysis (TGA) and Differential Scanning Calorimetry Analysis (DSC) (5 mW) thermograms of Form A of phosphate salt of Compound (I), wherein the molar ratio between Compound (I) and phosphoric acid is 1:1.

FIG. 1C shows the Differential Scanning Calorimetry Analysis (DSC) (10 mW) thermogram of Form A of phosphate salt of Compound (I), wherein the molar ratio between Compound (I) and phosphoric acid is 1:1.

FIGS. 2A and 2B (zoom on y-axis) show the X-ray Powder Diffraction (XRPD) pattern of Form G of phosphate salt of Compound (I), wherein the molar ratio between Compound (I) and phosphoric acid is 1:1.

FIG. 2C shows the Thermogravimetric Analysis (TGA) and Differential Scanning Calorimetry Analysis (DSC) thermograms of Form G of phosphate salt of Compound (I), wherein the molar ratio between Compound (I) and phosphoric acid is 1:1.

FIG. 2D shows the Differential Scanning Calorimetry Analysis (DSC) thermogram of Form G of phosphate salt of Compound (I), wherein the molar ratio between Compound (I) and phosphoric acid is 1:1.

FIG. 3 shows the X-ray Powder Diffraction (XRPD) pattern of Form O of phosphate salt of Compound (I), wherein the molar ratio between Compound (I) and phosphoric acid is 1:1.

FIG. 4A shows the X-ray Powder Diffraction (XRPD) pattern of Form 1-A of a besylate salt of Compound (I), wherein the molar ratio between Compound (I) and benzene sulfonic acid is 1:2.

FIG. 4B shows the Thermogravimetric Analysis (TGA) and Differential Scanning Calorimetry Analysis (DSC) thermograms of Form 1-A of a besylate salt of Compound (I), wherein the molar ratio between Compound (I) and benzene sulfonic acid is 1:2.

FIG. 4C shows the Differential Scanning Calorimetry Analysis (DSC) thermogram of Form 1-A of a besylate salt of Compound (I), wherein the molar ratio between Compound (I) and benzene sulfonic acid is 1:2.

FIG. 5 shows the X-ray Powder Diffraction (XRPD) pattern of Form 1-B of a besylate salt of Compound (I), wherein the molar ratio between Compound (I) and benzene sulfonic acid is 1:2.

FIG. 6A shows the X-ray Powder Diffraction (XRPD) pattern of Form 2-A of a benzoate salt of Compound (I), wherein the molar ratio between Compound (I) and benzoic acid is 1:1.

FIG. 6B shows the Thermogravimetric Analysis (TGA) and Differential Scanning Calorimetry Analysis (DSC) thermograms of Form 2-A of a benzoate salt of Compound (I), wherein the molar ratio between Compound (I) and benzoic acid is 1:1.

FIG. 6C shows the Differential Scanning Calorimetry Analysis (DSC) thermogram of Form 2-A of a benzoate salt of Compound (I), wherein the molar ratio between Compound (I) and benzoic acid is 1:1.

FIG. 7 shows the X-ray Powder Diffraction (XRPD) pattern of Form 2-B of a benzoate salt of Compound (I), wherein the molar ratio between Compound (I) and benzoic acid is 1:1.

FIG. 8A shows the X-ray Powder Diffraction (XRPD) pattern of Form 9-A of a sulfate salt of Compound (I), wherein the molar ratio between Compound (I) and sulfuric acid is 1:1.

FIG. 8B shows the Differential Scanning Calorimetry Analysis (DSC) thermogram of Form 9-A of a sulfate salt of Compound (I), wherein the molar ratio between Compound (I) and sulfuric acid is 1:1.

FIG. 9A shows the X-ray Powder Diffraction (XRPD) pattern of Form FB-A-0 of free base of Compound (I).

FIG. 9B shows the Thermogravimetric Analysis (TGA) and Differential Scanning Calorimetry Analysis (DSC) thermograms of crystalline free base of Compound (I).

FIG. 9C shows the Differential Scanning Calorimetry Analysis (DSC) thermogram of crystalline free base of Compound (I).

FIG. 10 shows the X-ray Powder Diffraction (XRPD) pattern of Form FB-A-1 of free base of Compound (I).

FIG. 11 shows the X-ray Powder Diffraction (XRPD) pattern of Form FB-A-2 of free base of Compound (I).

FIG. 12 shows the X-ray Powder Diffraction (XRPD) pattern of amorphous phosphate salt of Compound (I).

FIG. 13 shows DSC thermogram of amorphous phosphate salt of Compound (I), lyophilized.

FIG. 14 shows the X-ray Powder Diffraction (XRPD) pattern of amorphous Compound (I) free base prepared from the lyophilization of crystalline solid in various solvent systems. (1) Amorphous from ACN:water (7:3 vol.); (2) Amorphous (L.C) from t-BuOH; (3) Amorphous from t-BuOH:water (9:1 vol.).

FIG. 15 shows the X-ray Powder Diffraction (XRPD) pattern of free base of Compound (I) prepared from Example 10. Instrument: Bruker D8 Advance X-Ray Diffractometer, X-ray Source: Cu/K, ( [symbol] =1.54056 Å), Working electricity: 40 kV, 40 mA, Starting angle: 4°; stop angle: 40°, Increment: 0.05 deg./step, Scanspeed: 0.5 sec/step.

### DETAILED DESCRIPTION

The present disclosure is directed to: i) pharmaceutically acceptable salts of Compound (I); ii) novel solid forms of the free base of Compound (I) and novel solid forms of pharmaceutically acceptable salts of Compound (I), including unsolvated forms, solvated forms, amorphous forms, and crystalline forms (hereinafter collectively referred to as “Salt and Solid Forms of the disclosure”); and iii) methods of use and preparation of the Salt and Solid Forms of the disclosure.

### Phosphate Salt of Compound (I)

In one aspect, the present disclosure provides a phosphate salt of Compound (I), wherein the molar ratio between Compound (I) and phosphoric acid is 1:1.

In some embodiments, the phosphonate salt is crystalline. In some embodiments, the phosphate salt is in a single crystalline form.

In some embodiments, the phosphate salt is unsolvated. In other embodiments, the phosphate salt is solvated.

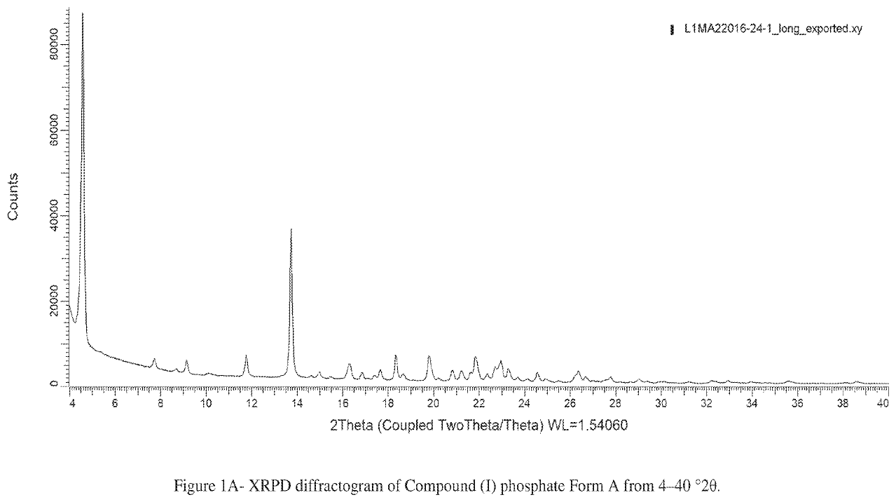

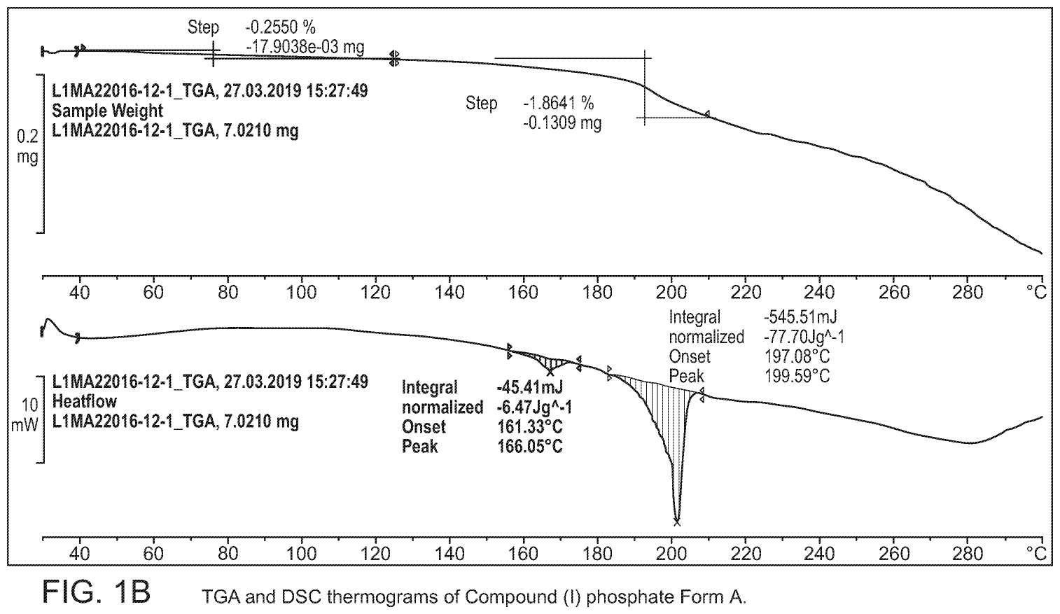

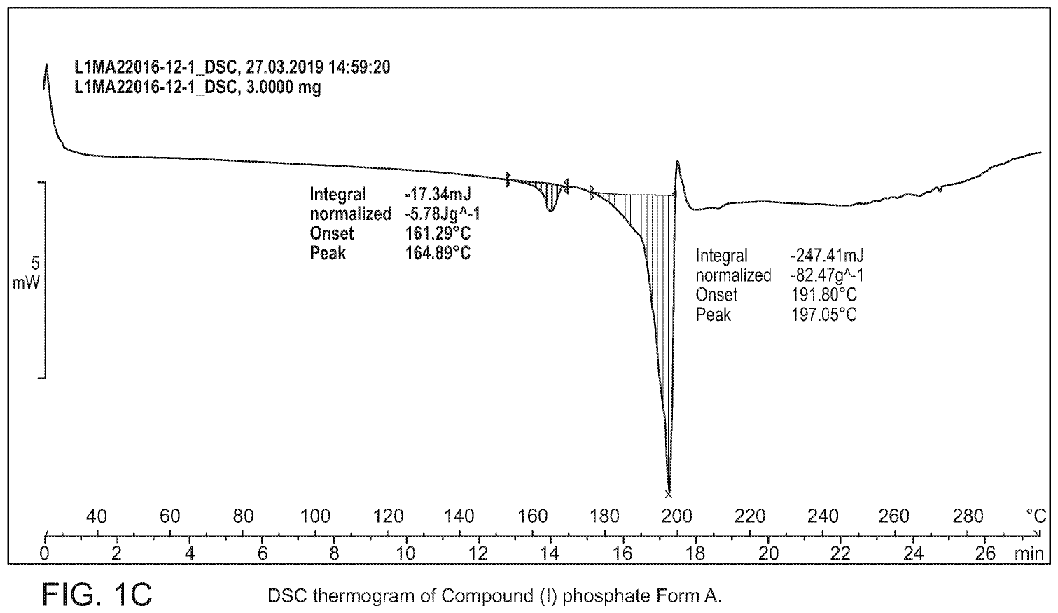

In some embodiments, the present disclosure provides crystalline Form A of phosphate salt of Compound (I). The XRPD pattern and peaks are shown in FIG. 1A. The Thermogravimetric Analysis (TGA) and Differential Scanning Calorimetry Analysis (DSC) thermograms are shown in FIG. 1B.

TABLE 1
Peak list for Compound (I) phosphate Form A.

| 2-theta (deg) | d-Spacing (ang.) | Relative Intensity |
|---|---|---|
| 4.58 | 19.29 | 100 |
| 7.72 | 11.44 | 3 |
| 9.15 | 9.66 | 4 |
| 11.76 | 7.52 | 7 |
| 13.73 | 6.44 | 47 |
| 16.30 | 5.43 | 5 |
| 17.66 | 5.02 | 3 |
| 18.34 | 4.83 | 8 |
| 19.82 | 4.48 | 7 |
| 20.84 | 4.26 | 3 |
| 21.22 | 4.18 | 3 |
| 21.84 | 4.07 | 7 |
| 22.71 | 3.91 | 4 |
| 22.96 | 3.87 | 6 |
| 23.29 | 3.82 | 4 |
| 24.55 | 3.62 | 3 |
| 26.37 | 3.38 | 3 |

Only peaks with relative intensity 3 or greater reported.

TABLE 2
Condensed peak list #1 for Compound (I) phosphate Form A.

| 2-theta (deg) | d-Spacing (ang.) | Relative Intensity |
|---|---|---|
| 4.58 | 19.29 | 100 |
| 9.15 | 9.66 | 4 |
| 11.76 | 7.52 | 7 |
| 13.73 | 6.44 | 47 |
| 16.30 | 5.43 | 5 |
| 18.34 | 4.83 | 8 |
| 19.82 | 4.48 | 7 |
| 21.84 | 4.07 | 7 |

TABLE 3
Condensed peak list #2 for Compound (I) phosphate Form A.

| 2-theta (deg) | d-Spacing (ang.) | Relative Intensity |
|---|---|---|
| 4.58 | 19.29 | 100 |
| 11.76 | 7.52 | 7 |
| 13.73 | 6.44 | 47 |
| 18.34 | 4.83 | 8 |
| 19.82 | 4.48 | 7 |
| 21.84 | 4.07 | 7 |

TABLE 4
Condensed peak list #3 for Compound (I) phosphate Form A.

| 2-theta (deg) | d-Spacing (ang.) | Relative Intensity |
|---|---|---|
| 4.58 | 19.29 | 100 |
| 11.76 | 7.52 | 7 |
| 13.73 | 6.44 | 47 |
| 18.34 | 4.83 | 8 |
| 19.82 | 4.48 | 7 |

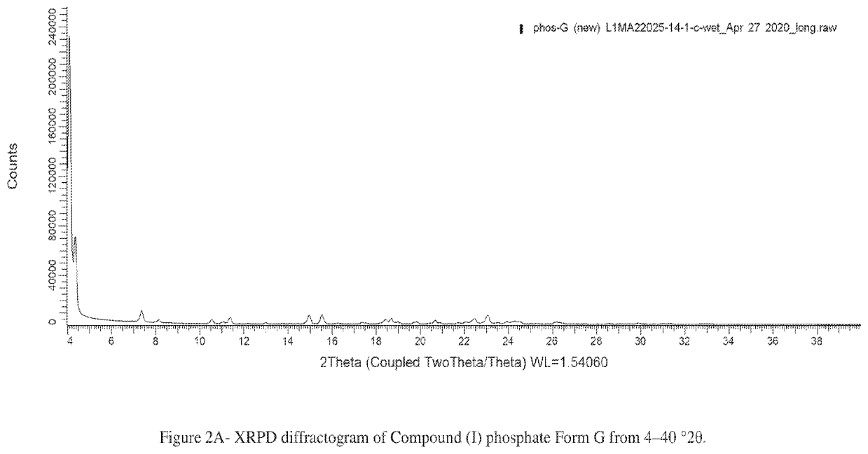

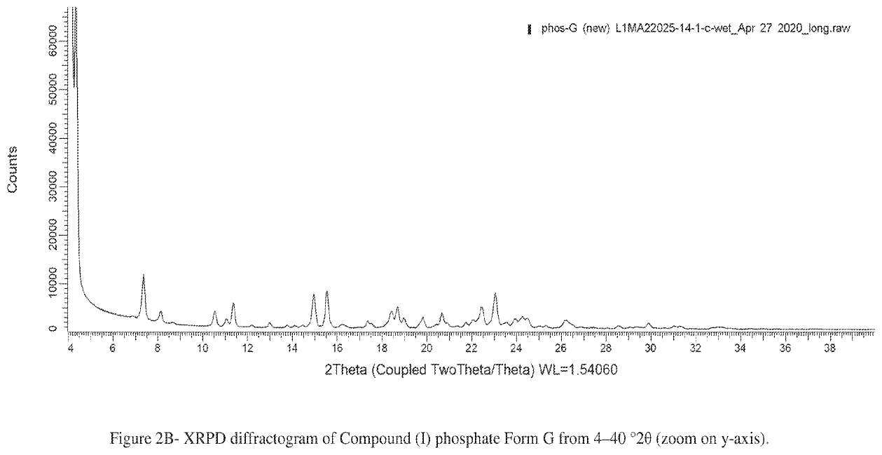

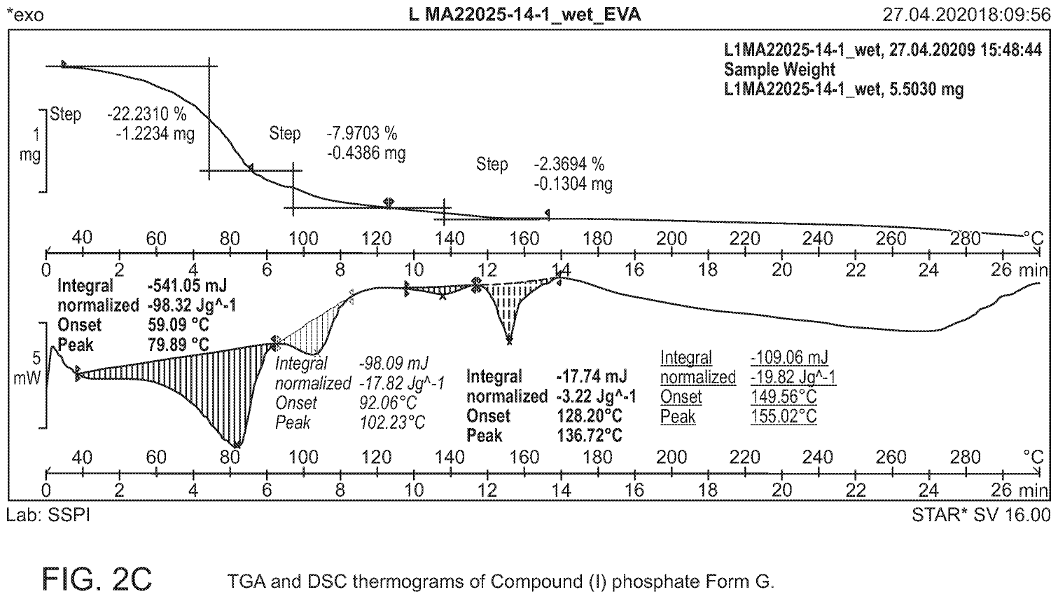

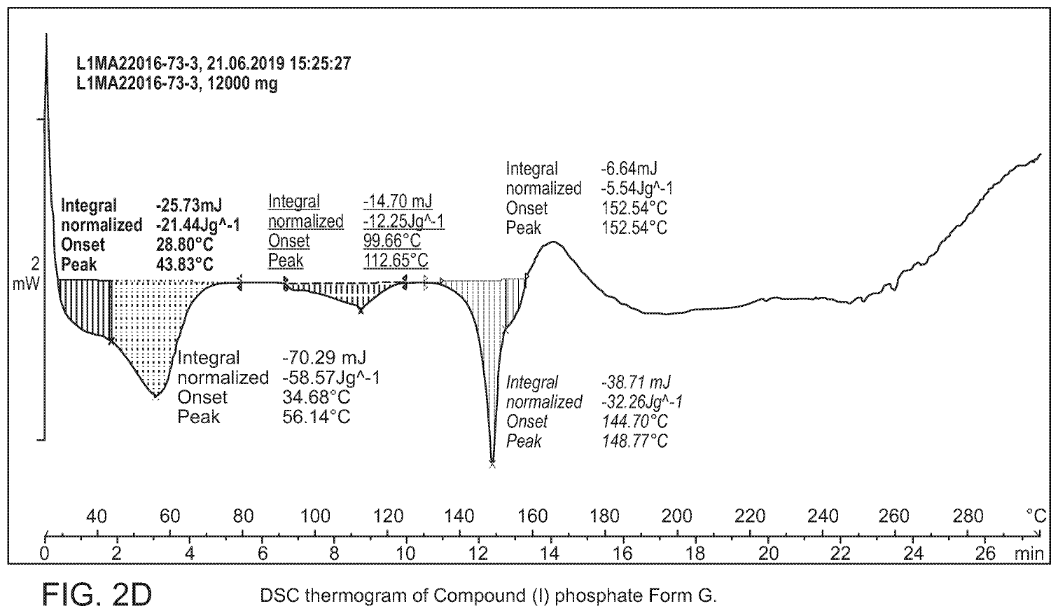

In some embodiments, the present disclosure provides crystalline Form G of phosphate salt of Compound (I). The XRPD pattern and peaks are shown in FIGS. 2A and 2B. The Thermogravimetric Analysis (TGA) and Differential Scanning Calorimetry Analysis (DSC) thermograms are shown in FIGS. 2C and 2D.

TABLE 5
Peak list for Compound (I) phosphate Form G.

| 2-theta (deg) | d-Spacing (ang.) | Relative Intensity |
|---|---|---|
| 4.09 | 21.60 | 100.0 |
| 4.35 | 20.31 | 27.4 |
| 7.37 | 11.99 | 4.4 |
| 8.13 | 10.87 | 1.0 |
| 10.55 | 8.38 | 1.5 |
| 11.06 | 7.99 | 0.7 |
| 11.37 | 7.77 | 2.3 |
| 13.00 | 6.81 | 0.5 |
| 14.97 | 5.91 | 3.2 |
| 15.55 | 5.69 | 3.6 |
| 18.69 | 4.74 | 2.0 |
| 19.01 | 4.67 | 0.9 |
| 19.84 | 4.47 | 1.0 |
| 20.67 | 4.29 | 1.4 |
| 22.45 | 3.96 | 1.9 |
| 23.01 | 3.85 | 3.3 |
| 23.95 | 3.71 | 0.8 |
| 24.33 | 3.65 | 0.8 |
| 26.21 | 3.40 | 0.8 |
| 29.90 | 2.99 | 0.5 |

Only peaks with relative intensity 0.5 or greater reported.

TABLE 6
Condensed peak list #1 for Compound (I) phosphate Form G.

| 2-theta (deg) | d-Spacing (ang.) | Relative Intensity |
|---|---|---|
| 4.09 | 21.60 | 100 |
| 4.35 | 20.31 | 27 |
| 7.37 | 11.99 | 4 |
| 8.13 | 10.87 | 1 |
| 10.55 | 8.38 | 2 |
| 11.37 | 7.77 | 2 |
| 14.97 | 5.91 | 3 |
| 15.55 | 5.69 | 4 |
| 18.69 | 4.74 | 2 |
| 20.67 | 4.29 | 1 |
| 22.45 | 3.96 | 2 |
| 23.01 | 3.85 | 3 |

Only peaks with relative intensity 0.5 or greater reported.

TABLE 7
Condensed peak list #2 for Compound (I) phosphate Form G.

| 2-theta (deg) | d-Spacing (ang.) | Relative Intensity |
|---|---|---|
| 4.09 | 21.60 | 100 |
| 4.35 | 20.31 | 27 |
| 7.37 | 11.99 | 4 |
| 14.97 | 5.91 | 3 |
| 15.55 | 5.69 | 4 |
| 18.69 | 4.74 | 2 |
| 22.45 | 3.96 | 2 |
| 23.01 | 3.85 | 3 |

TABLE 8
Condensed peak list #3 for Compound (I) phosphate Form G.

| 2-theta (deg) | d-Spacing (ang.) | Relative Intensity |
|---|---|---|
| 4.09 | 21.60 | 100 |
| 4.35 | 20.31 | 27 |
| 7.37 | 11.99 | 4 |
| 15.55 | 5.69 | 4 |
| 23.01 | 3.85 | 3 |

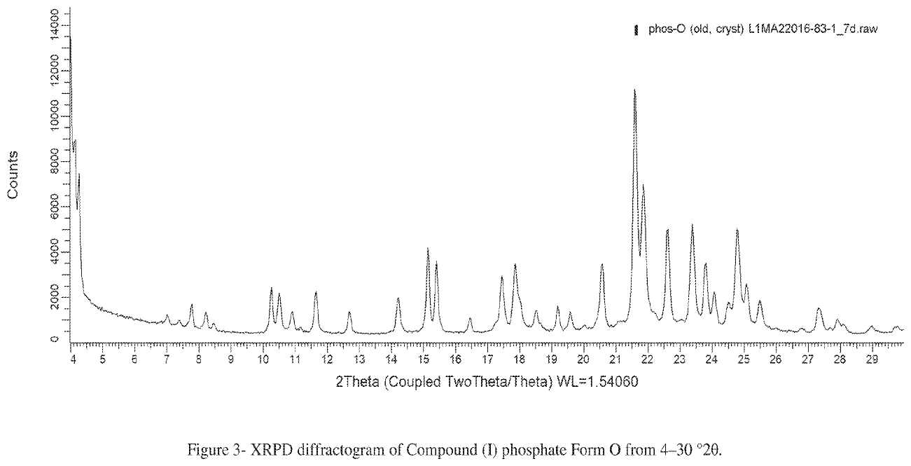

In some embodiments. the present disclosure provides crystalline Form O of a phosphate salt of Compound (I). The XRPD pattern and peaks are shown in FIG. 3.

TABLE 9
Peak list for Compound (I) phosphate Form O.

| 2-theta (deg) | d-Spacing (ang.) | Relative Intensity |
|---|---|---|
| 4.15 | 21.29 | 64 |
| 4.27 | 20.69 | 52 |
| 7.78 | 11.36 | 8 |
| 8.22 | 10.74 | 6 |
| 10.26 | 8.61 | 17 |
| 10.51 | 8.41 | 15 |
| 10.91 | 8.10 | 8 |
| 11.65 | 7.59 | 16 |
| 12.70 | 6.97 | 8 |
| 14.22 | 6.22 | 14 |
| 15.15 | 5.84 | 35 |
| 15.41 | 5.74 | 30 |
| 16.46 | 5.38 | 6 |
| 17.45 | 5.08 | 23 |
| 17.86 | 4.96 | 27 |
| 18.52 | 4.79 | 8 |
| 19.20 | 4.62 | 10 |
| 19.59 | 4.53 | 7 |
| 20.57 | 4.31 | 27 |
| 21.60 | 4.11 | 100 |
| 21.86 | 4.06 | 58 |
| 22.10 | 4.02 | 6 |
| 22.61 | 3.93 | 43 |
| 23.39 | 3.80 | 44 |
| 23.80 | 3.74 | 27 |
| 24.08 | 3.69 | 15 |
| 24.52 | 3.63 | 11 |
| 24.79 | 3.59 | 42 |
| 25.08 | 3.55 | 19 |
| 25.50 | 3.49 | 12 |
| 27.34 | 3.26 | 10 |
| 27.92 | 3.19 | 6 |

Only peaks with relative intensity 5 or greater reported.

TABLE 10
Condensed peak list #1 for Compound (I) phosphate Form O.

| 2-theta (deg) | d-Spacing (ang.) | Relative Intensity |
|---|---|---|
| 4.15 | 21.29 | 64 |
| 4.27 | 20.69 | 52 |
| 11.65 | 7.59 | 16 |
| 15.15 | 5.84 | 35 |
| 15.41 | 5.74 | 30 |
| 17.86 | 4.96 | 27 |
| 20.57 | 4.31 | 27 |
| 21.60 | 4.11 | 100 |
| 21.86 | 4.06 | 58 |
| 22.61 | 3.93 | 43 |
| 23.39 | 3.80 | 44 |
| 24.79 | 3.59 | 42 |

TABLE 11
Condensed peak list #2 for Compound (I) phosphate Form O.

| 2-theta (deg) | d-Spacing (ang.) | Relative Intensity |
|---|---|---|
| 4.15 | 21.29 | 64 |
| 4.27 | 20.69 | 52 |
| 15.15 | 5.84 | 35 |
| 15.41 | 5.74 | 30 |
| 21.60 | 4.11 | 100 |
| 21.86 | 4.06 | 58 |
| 22.61 | 3.93 | 43 |
| 24.79 | 3.59 | 42 |

TABLE 12
Condensed peak list #3 for Compound (I) phosphate Form O.

| 2-theta (deg) | d-Spacing (ang.) | Relative Intensity |
|---|---|---|
| 4.15 | 21.29 | 64 |
| 15.15 | 5.84 | 35 |
| 21.60 | 4.11 | 100 |
| 21.86 | 4.06 | 58 |
| 22.61 | 3.93 | 43 |

### Besylate Salt of Compound (I)

In one aspect, the present disclosure provides a besylate salt of Compound (1), wherein the molar ratio between Compound (1) and benzene sulfonic acid is 1:2.

In some embodiments, the besylate salt is crystalline. In some embodiments, the besylate salt is in a single crystalline form.

In some embodiments, the besylate salt is unsolvated. In other embodiments, the besylate salt is solvated.

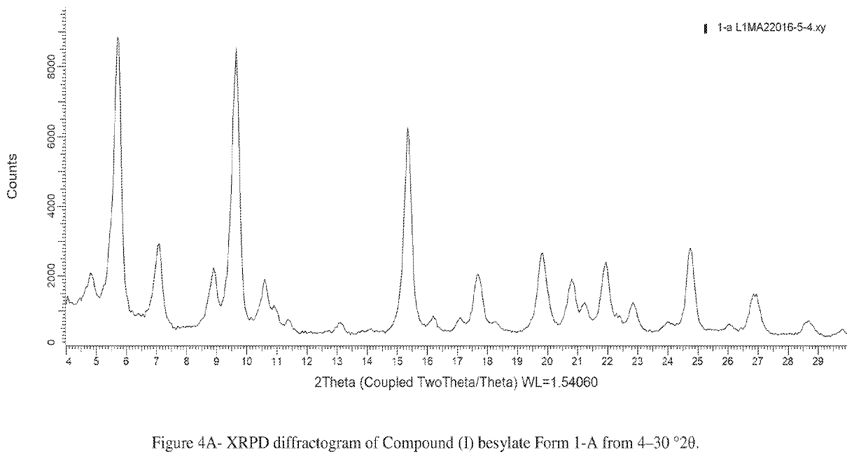

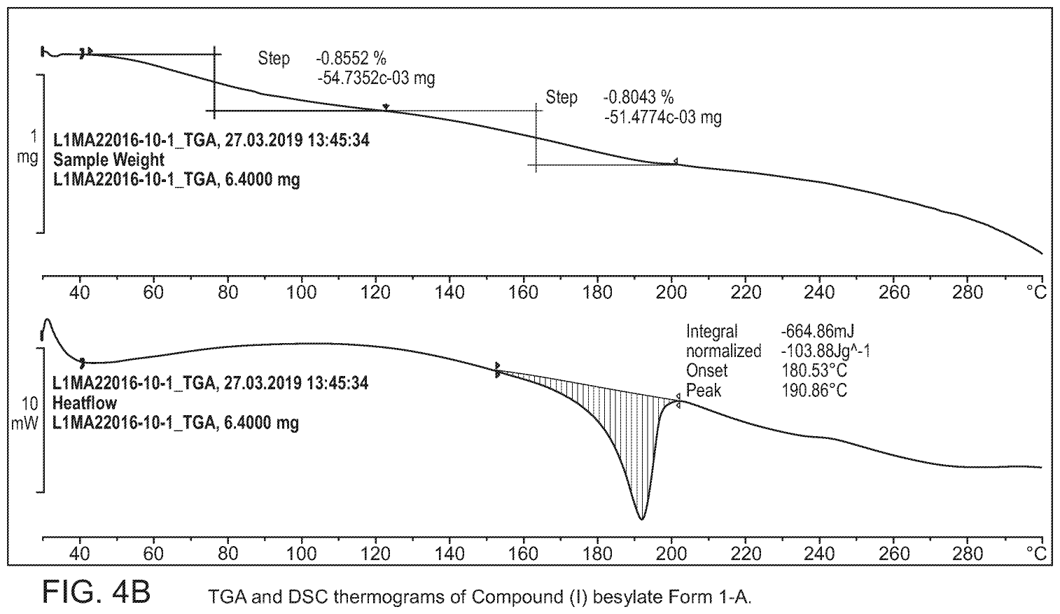

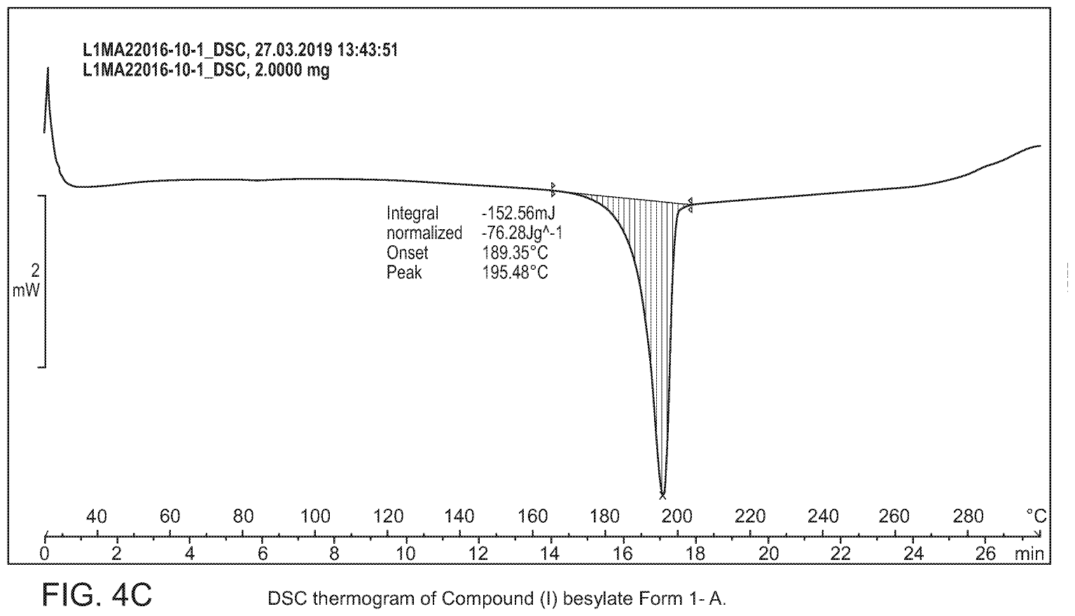

In some embodiments, the present disclosure provides crystalline Form 1-A of a besylate salt of Compound (I). The XRPD pattern and peaks are shown in FIG. 4A. The Thermogravimetric Analysis (TGA) and Differential Scanning Calorimetry Analysis (DSC) thermograms are shown in FIGS. 4B and 4C.

TABLE 13
Peak list for Compound (I) besylate Form 1-A.

| 2-theta (deg) | d-Spacing (ang.) | Relative Intensity |
|---|---|---|
| 4.83 | 18.28 | 12 |
| 5.72 | 15.43 | 99 |
| 7.08 | 12.48 | 28 |
| 8.90 | 9.93 | 21 |
| 9.65 | 9.16 | 100 |
| 10.60 | 8.34 | 18 |
| 10.88 | 8.12 | 9 |
| 15.36 | 5.76 | 72 |
| 16.22 | 5.46 | 5 |
| 17.69 | 5.01 | 20 |
| 19.82 | 4.47 | 28 |
| 20.82 | 4.26 | 18 |
| 21.23 | 4.18 | 9 |
| 21.94 | 4.05 | 24 |
| 22.85 | 3.89 | 10 |
| 24.76 | 3.59 | 30 |
| 26.91 | 3.31 | 13 |
| 28.68 | 3.11 | 5 |

Only peaks with relative intensity 5 or greater reported.

TABLE 14
Condensed peak list #1 for Compound (I) besylate Form 1-A.

| 2-theta (deg) | d-Spacing (ang.) | Relative Intensity |
|---|---|---|
| 5.72 | 15.43 | 99 |
| 7.08 | 12.48 | 28 |
| 8.90 | 9.93 | 21 |
| 9.65 | 9.16 | 100 |
| 10.60 | 8.34 | 18 |
| 15.36 | 5.76 | 72 |
| 17.69 | 5.01 | 20 |
| 19.82 | 4.47 | 28 |
| 20.82 | 4.26 | 18 |
| 21.94 | 4.05 | 24 |
| 22.85 | 3.89 | 10 |
| 24.76 | 3.59 | 30 |

TABLE 15
Condensed peak list #2 for Compound (I) besylate Form 1-A.

| 2-theta (deg) | d-Spacing (ang.) | Relative Intensity |
|---|---|---|
| 5.72 | 15.43 | 99 |
| 7.08 | 12.48 | 28 |
| 8.90 | 9.93 | 21 |
| 9.65 | 9.16 | 100 |
| 15.36 | 5.76 | 72 |
| 19.82 | 4.47 | 28 |
| 21.94 | 4.05 | 24 |
| 24.76 | 3.59 | 30 |

TABLE 16
Condensed peak list #3 for Compound (I) besylate Form 1-A.

| 2-theta (deg) | d-Spacing (ang.) | Relative Intensity |
|---|---|---|
| 5.72 | 15.43 | 99 |
| 7.08 | 12.48 | 28 |
| 9.65 | 9.16 | 100 |
| 15.36 | 5.76 | 72 |
| 24.76 | 3.59 | 30 |

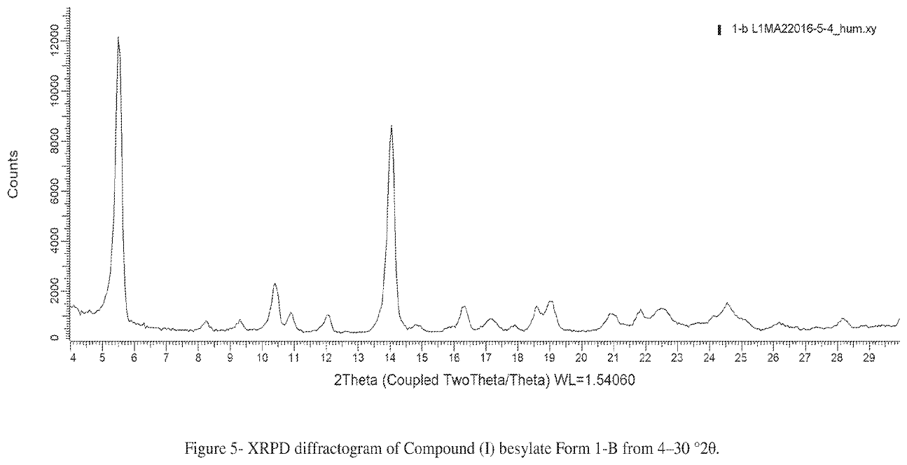

In some embodiments, the present disclosure provides crystalline Form 1-B of a besylate salt of Compound (I). The XRPD pattern and peaks are shown in FIG. 5.

TABLE 17
Peak list for Compound (I) besylate Form 1-B.

| 2-theta (deg) | d-Spacing (ang.) | Relative Intensity |
|---|---|---|
| 5.51 | 16.02 | 100 |
| 10.41 | 8.49 | 17 |
| 10.90 | 8.11 | 7 |
| 12.05 | 7.34 | 6 |
| 14.04 | 6.30 | 73 |
| 16.31 | 5.43 | 8 |
| 18.61 | 4.76 | 8 |
| 19.03 | 4.66 | 10 |
| 20.93 | 4.24 | 6 |
| 21.84 | 4.07 | 7 |
| 22.50 | 3.95 | 7 |
| 24.57 | 3.62 | 9 |
| 24.57 | 3.62 | 9 |

Only peaks with relative intensity 5 or greater reported.

TABLE 18
Condensed peak list #1 for Compound (I) besylate Form 1-B.

| 2-theta (deg) | d-Spacing (ang.) | Relative Intensity |
|---|---|---|
| 5.51 | 16.02 | 100 |
| 10.41 | 8.49 | 17 |
| 10.90 | 8.11 | 7 |
| 14.04 | 6.30 | 73 |
| 16.31 | 5.43 | 8 |
| 18.61 | 4.76 | 8 |
| 19.03 | 4.66 | 10 |
| 20.93 | 4.24 | 6 |
| 21.84 | 4.07 | 7 |
| 24.57 | 3.62 | 9 |

TABLE 19
Condensed peak list #2 for Compound (I) besylate Form 1-B.

| 2-theta (deg) | d-Spacing (ang.) | Relative Intensity |
|---|---|---|
| 5.51 | 16.02 | 100 |
| 10.41 | 8.49 | 17 |
| 14.04 | 6.30 | 73 |
| 16.31 | 5.43 | 8 |
| 18.61 | 4.76 | 8 |
| 19.03 | 4.66 | 10 |

TABLE 20
Condensed peak list #3 for Compound (I) besylate Form 1-B.

| 2-theta (deg) | d-Spacing (ang.) | Relative Intensity |
|---|---|---|
| 5.51 | 16.02 | 100 |
| 10.41 | 8.49 | 17 |
| 14.04 | 6.30 | 73 |
| 16.31 | 5.43 | 8 |

### Benzoate Salt of Compound (I)

In one aspect, the present disclosure provides a benzoate salt of Compound (I), wherein the molar ratio between Compound (I) and benzoic acid is 1:1.

In some embodiments, the benzoate salt is crystalline. In some embodiments, the benzoate salt is in a single crystalline form.

In some embodiments, the benzoate salt is unsolvated. In other embodiments, the benzoate salt is solvated.

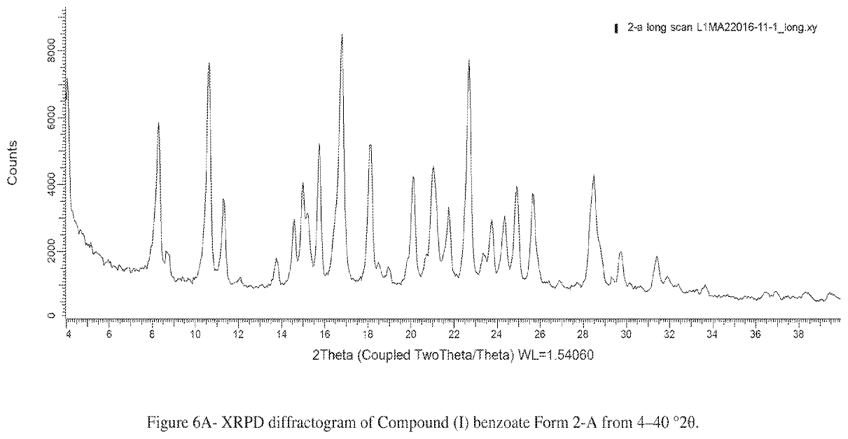

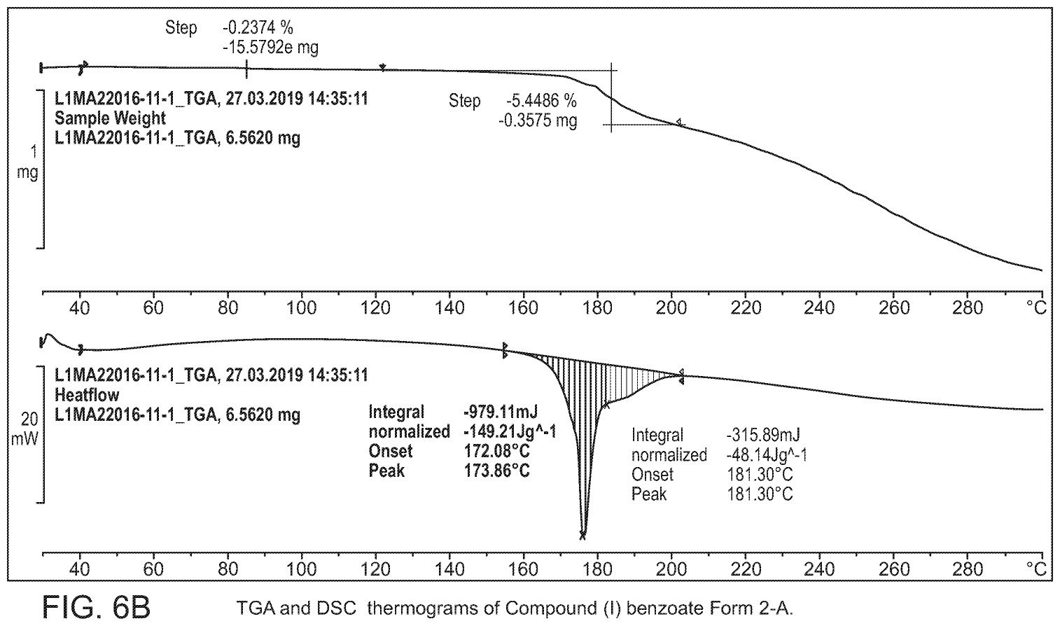

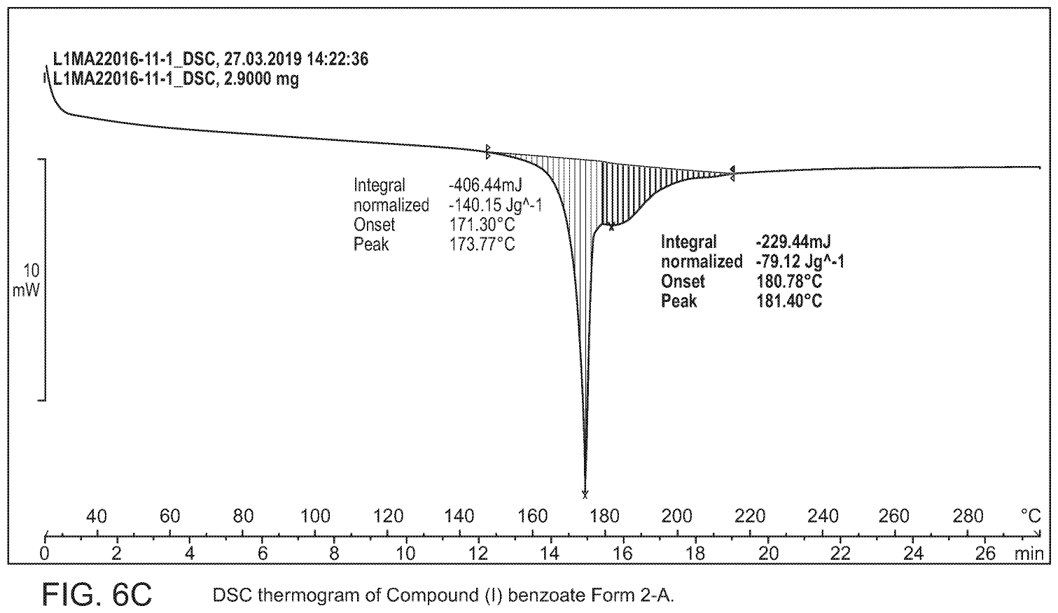

In some embodiments, the present disclosure provides crystalline Form 2-A of benzoate salt of Compound (I). The XRPD pattern and peaks are shown in FIG. 6A. The Thermogravimetric Analysis (TGA) and Differential Scanning Calorimetry Analysis (DSC) thermograms are shown in FIGS. 6B and 6C.

TABLE 21
Peak list for Compound (I) benzoate Form 2-A.

| 2-theta (deg) | d-Spacing (ang.) | Relative Intensity |
|---|---|---|
| 4.05 | 21.82 | 68 |
| 8.29 | 10.66 | 53 |
| 8.70 | 10.15 | 7 |
| 10.63 | 8.31 | 80 |
| 11.32 | 7.81 | 31 |
| 13.77 | 6.43 | 9 |
| 14.59 | 6.07 | 24 |
| 14.99 | 5.90 | 39 |
| 15.21 | 5.82 | 27 |
| 15.76 | 5.62 | 55 |
| 16.80 | 5.27 | 100 |
| 18.13 | 4.89 | 56 |
| 20.12 | 4.41 | 44 |
| 20.75 | 4.28 | 11 |
| 21.04 | 4.22 | 47 |
| 21.76 | 4.08 | 32 |
| 22.70 | 3.91 | 94 |
| 23.74 | 3.74 | 26 |
| 24.35 | 3.65 | 28 |
| 24.91 | 3.57 | 41 |
| 25.67 | 3.47 | 39 |
| 28.49 | 3.13 | 48 |
| 29.74 | 3.00 | 16 |
| 31.41 | 2.85 | 15 |
| 31.90 | 2.80 | 7 |
| 33.64 | 2.66 | 5 |

Only peaks with relative intensity 5 or greater reported.

TABLE 22
Condensed peak list #1 for Compound (I) benzoate Form 2-A.

| 2-theta (deg) | d-Spacing (ang.) | Relative Intensity |
|---|---|---|
| 4.05 | 21.82 | 68 |
| 8.29 | 10.66 | 53 |
| 10.63 | 8.31 | 80 |
| 14.99 | 5.90 | 39 |
| 15.76 | 5.62 | 55 |
| 16.80 | 5.27 | 100 |
| 18.13 | 4.89 | 56 |
| 20.12 | 4.41 | 44 |
| 21.04 | 4.22 | 47 |
| 22.70 | 3.91 | 94 |
| 25.67 | 3.47 | 39 |
| 28.49 | 3.13 | 48 |

TABLE 23
Condensed peak list #2 for Compound (I) benzoate Form 2-A.

| 2-theta (deg) | d-Spacing (ang.) | Relative Intensity |
|---|---|---|
| 4.05 | 21.82 | 68 |
| 8.29 | 10.66 | 53 |
| 10.63 | 8.31 | 80 |
| 16.80 | 5.27 | 100 |
| 18.13 | 4.89 | 56 |
| 22.70 | 3.91 | 94 |
| 28.49 | 3.13 | 48 |

TABLE 24
Condensed peak list #3 for Compound (I) benzoate Form 2-A.

| 2-theta (deg) | d-Spacing (ang.) | Relative Intensity |
|---|---|---|
| 4.05 | 21.82 | 68 |
| 10.63 | 8.31 | 80 |
| 16.80 | 5.27 | 100 |
| 18.13 | 4.89 | 56 |
| 22.70 | 3.91 | 94 |

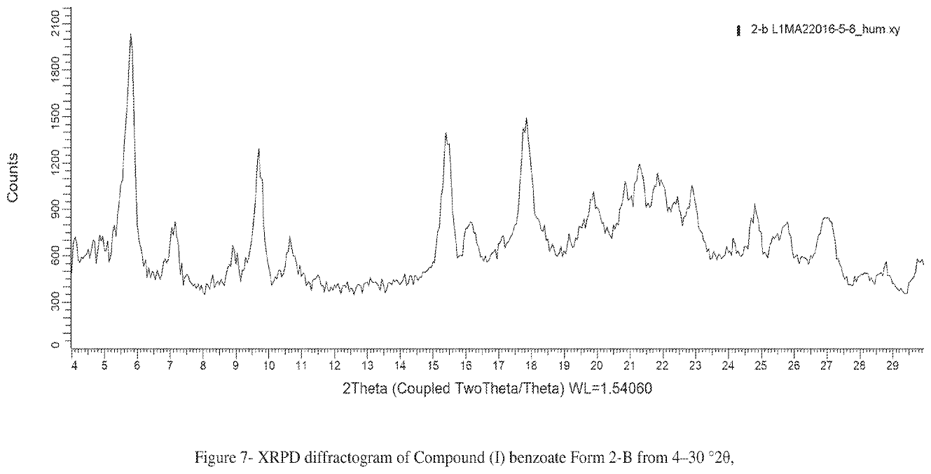

In some embodiments, the present disclosure provides crystalline Form 2-B of benzoate salt of Compound (I). The XRPD pattern and peaks are shown in FIG. 7.

TABLE 25
Peak list for Compound (I) benzoate Form 2-B.

| 2-theta (deg) | d-Spacing (ang.) | Relative Intensity |
|---|---|---|
| 5.79 | 15.24 | 100 |
| 7.11 | 12.43 | 21 |
| 8.91 | 9.92 | 17 |
| 9.71 | 9.10 | 56 |
| 10.65 | 8.30 | 21 |
| 15.43 | 5.74 | 54 |
| 16.14 | 5.49 | 18 |
| 17.82 | 4.97 | 54 |
| 19.86 | 4.47 | 22 |
| 20.86 | 4.25 | 28 |
| 21.30 | 4.17 | 36 |
| 21.88 | 4.06 | 29 |
| 22.41 | 3.96 | 20 |
| 22.90 | 3.88 | 28 |
| 24.81 | 3.59 | 23 |
| 25.75 | 3.46 | 17 |
| 26.99 | 3.30 | 24 |
| 28.79 | 3.10 | 11 |

Only peaks with relative intensity 5 or greater reported.

TABLE 26
Condensed peak list #1 for Compound (I) benzoate Form 2-B.

| 2-theta (deg) | d-Spacing (ang.) | Relative Intensity |
|---|---|---|
| 5.79 | 15.24 | 100 |
| 7.11 | 12.43 | 21 |
| 9.71 | 9.10 | 56 |
| 10.65 | 8.30 | 21 |
| 15.43 | 5.74 | 54 |
| 16.14 | 5.49 | 18 |
| 17.82 | 4.97 | 54 |
| 22.90 | 3.88 | 28 |
| 24.81 | 3.59 | 23 |
| 26.99 | 3.30 | 24 |

TABLE 27
Condensed peak list #2 for Compound (I) benzoate Form 2-B.

| 2-theta (deg) | d-Spacing (ang.) | Relative Intensity |
|---|---|---|
| 5.79 | 15.24 | 100 |
| 7.11 | 12.43 | 21 |
| 9.71 | 9.10 | 56 |
| 10.65 | 8.30 | 21 |
| 15.43 | 5.74 | 54 |
| 17.82 | 4.97 | 54 |
| 22.90 | 3.88 | 28 |

TABLE 28
Condensed peak list #3 for Compound (I) benzoate Form 2-B.

| 2-theta (deg) | d-Spacing (ang.) | Relative Intensity |
|---|---|---|
| 5.79 | 15.24 | 100 |
| 9.71 | 9.10 | 56 |
| 15.43 | 5.74 | 54 |
| 17.82 | 4.97 | 54 |

### Sulfate Salt of Compound (I)

In one aspect, the present disclosure provides a sulfate salt of Compound (I), wherein the molar ratio between Compound (I) and sulfuric acid is 1:1.

In some embodiments, the sulfate salt is crystalline. In some embodiments, the sulfate salt is in a single crystalline form.

In some embodiments, the sulfate salt is unsolvated. In other embodiments, the sulfate salt is solvated.

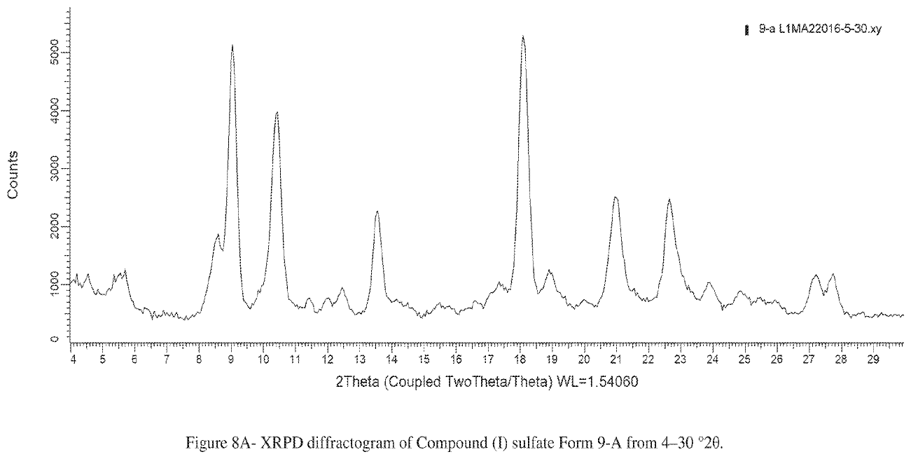

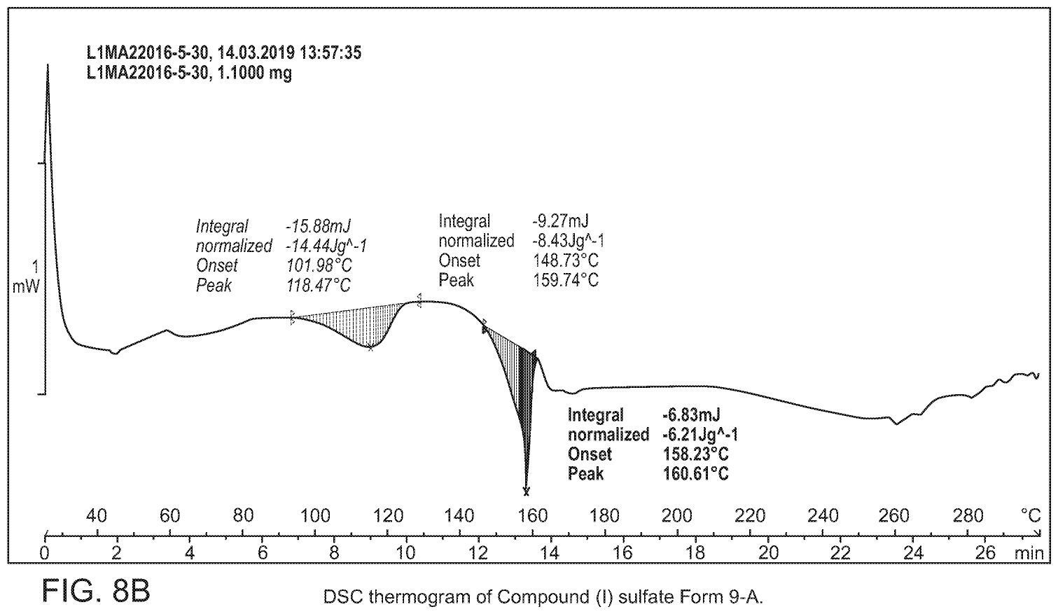

In some embodiments, the present disclosure provides crystalline Form 9-A of a sulfate salt of Compound (I). The XRPD pattern and peaks are shown in FIG. 8A. The Differential Scanning Calorimetry Analysis (DSC) thermogram is shown in FIG. 8B.

TABLE 29
Peak list for Compound (I) sulfate Form 9-A.

| 2-theta (deg) | d-Spacing (ang.) | Relative Intensity |
|---|---|---|
| 4.53 | 19.48 | 5 |
| 5.55 | 15.90 | 10 |
| 8.58 | 10.30 | 29 |
| 9.04 | 9.77 | 99 |
| 10.43 | 8.48 | 74 |
| 11.43 | 7.74 | 5 |
| 12.01 | 7.36 | 5 |
| 12.45 | 7.10 | 9 |
| 13.56 | 6.53 | 38 |
| 17.37 | 5.10 | 10 |
| 18.10 | 4.90 | 100 |
| 18.93 | 4.68 | 13 |
| 20.99 | 4.23 | 39 |
| 22.66 | 3.92 | 39 |
| 23.89 | 3.72 | 9 |
| 24.86 | 3.58 | 6 |
| 27.21 | 3.27 | 14 |
| 27.73 | 3.21 | 14 |

Only peaks with relative intensity 5 or greater reported.

TABLE 30
Condensed peak list #1 for Compound (I) sulfate Form 9-A.

| 2-theta (deg) | d-Spacing (ang.) | Relative Intensity |
|---|---|---|
| 8.58 | 10.30 | 29 |
| 9.04 | 9.77 | 99 |
| 10.43 | 8.48 | 74 |
| 13.56 | 6.53 | 38 |
| 17.37 | 5.10 | 10 |
| 18.10 | 4.90 | 100 |
| 18.93 | 4.68 | 13 |
| 20.99 | 4.23 | 39 |
| 22.66 | 3.92 | 39 |
| 23.89 | 3.72 | 9 |
| 27.21 | 3.27 | 14 |
| 27.73 | 3.21 | 14 |

TABLE 31
Condensed peak list #2 for Compound (I) sulfate Form 9-A.

| 2-theta (deg) | d-Spacing (ang.) | Relative Intensity |
|---|---|---|
| 8.58 | 10.30 | 29 |
| 9.04 | 9.77 | 99 |
| 10.43 | 8.48 | 74 |
| 13.56 | 6.53 | 38 |
| 18.10 | 4.90 | 100 |
| 20.99 | 4.23 | 39 |
| 22.66 | 3.92 | 39 |
| 27.73 | 3.21 | 14 |

TABLE 32
Condensed peak list #3 for Compound (I) sulfate Form 9-A.

| 2-theta (deg) | d-Spacing (ang.) | Relative Intensity |
|---|---|---|
| 9.04 | 9.77 | 99 |
| 10.43 | 8.48 | 74 |
| 13.56 | 6.53 | 38 |
| 18.10 | 4.90 | 100 |
| 20.99 | 4.23 | 39 |

### Free Base of Compound (I)

In one aspect, the present disclosure provides free base of Compound (I).

In some embodiments, the free base of Compound (I) is in an amorphous form.

In some embodiments, the free base of Compound (I) is crystalline. In some embodiments, the free base of Compound (I) is in a single crystalline form.

In some embodiments, the free base of Compound (I) is unsolvated. In other embodiments, the free base of Compound (I) is solvated.

In some embodiments, the present disclosure provides a crystalline family of Form FB-A of free base of Compound (I), which comprises Form FB-A-0 (monohydrate), Form FB-A-1 (metastable hydrate) and Form FB-A-2 (dehydrate).

As described herein, a crystalline family is a group of crystalline forms including the same crystalline form which can be described as “interconverting” because of different amounts of water residing in crystal lattice. The different amount of water results in slight peak shifts in the XRPD.

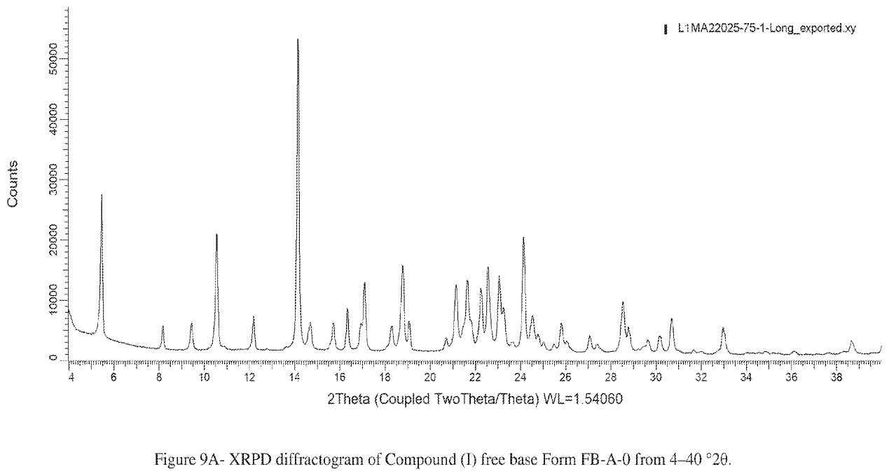

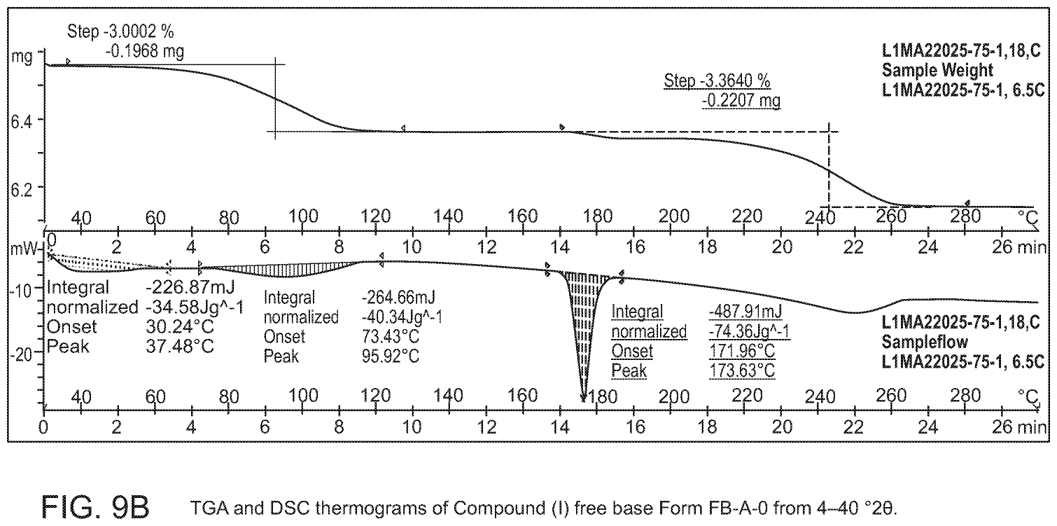

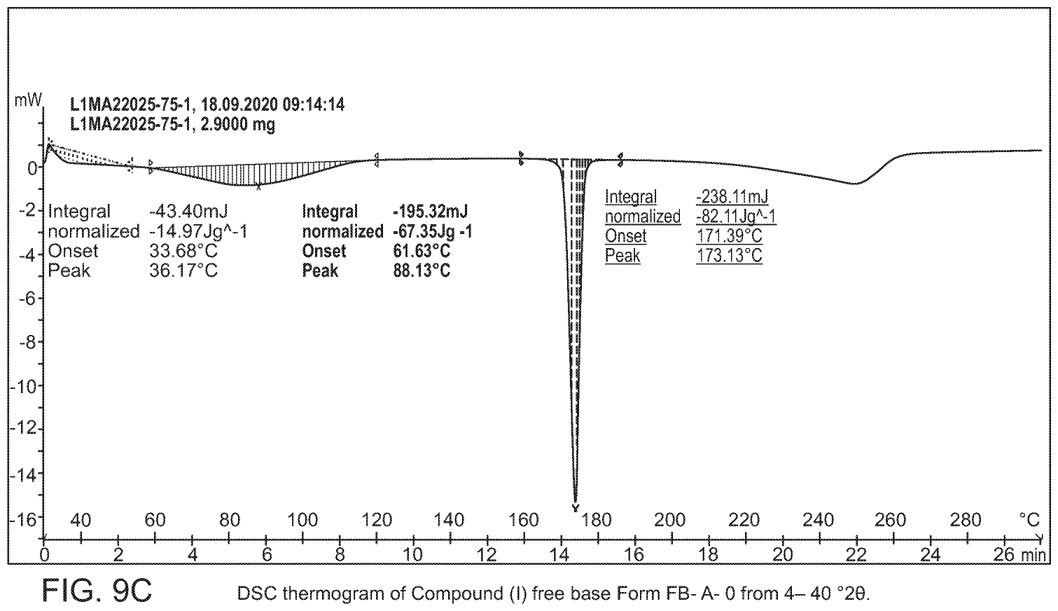

The crystalline family of Form FB-A-0 is found to be a channel hydrate, which can accommodate different amounts of water in the crystal lattice depending on the ambient humidity. The XRPD pattern and peaks are shown in FIG. 9A.

TABLE 33
Peak list for Compound (I) free base Form FB-A-0.

| 2-theta (deg) | d-Spacing (ang.) | Relative Intensity |
|---|---|---|
| 5.45 | 16.20 | 46 |
| 8.16 | 10.83 | 8 |
| 9.44 | 9.36 | 9 |
| 10.55 | 8.38 | 38 |
| 12.18 | 7.26 | 11 |
| 14.15 | 6.25 | 100 |
| 14.69 | 6.02 | 9 |
| 15.71 | 5.64 | 9 |
| 16.34 | 5.42 | 14 |
| 16.94 | 5.23 | 9 |
| 17.09 | 5.18 | 22 |
| 18.30 | 4.84 | 8 |
| 18.78 | 4.72 | 28 |
| 19.07 | 4.65 | 9 |
| 21.15 | 4.20 | 21 |
| 21.65 | 4.10 | 23 |
| 22.25 | 3.99 | 20 |
| 22.56 | 3.94 | 27 |
| 23.09 | 3.85 | 22 |
| 23.25 | 3.82 | 14 |
| 24.14 | 3.68 | 37 |
| 24.54 | 3.63 | 11 |
| 24.78 | 3.59 | 5 |
| 25.81 | 3.45 | 9 |
| 27.06 | 3.29 | 5 |
| 28.54 | 3.13 | 16 |
| 28.78 | 3.10 | 8 |
| 30.18 | 2.96 | 5 |
| 30.69 | 2.91 | 11 |
| 32.97 | 2.71 | 9 |

Only peaks with relative intensity 5 or greater reported.

TABLE 34
Condensed peak list # 1 for Compound (I) free base Form FB-A-0.

| 2-theta (deg) | d-Spacing (ang.) | Relative Intensity |
|---|---|---|
| 5.45 | 16.20 | 46 |
| 10.55 | 8.38 | 38 |
| 12.18 | 7.26 | 11 |
| 14.15 | 6.25 | 100 |
| 17.09 | 5.18 | 22 |
| 18.78 | 4.72 | 28 |
| 21.15 | 4.20 | 21 |
| 22.25 | 3.99 | 20 |
| 22.56 | 3.94 | 27 |
| 23.09 | 3.85 | 22 |
| 24.14 | 3.68 | 37 |
| 28.54 | 3.13 | 16 |

TABLE 35
Condensed peak list # 2 for Compound (I) free base Form FB-A-0.

| 2-theta (deg) | d-Spacing (ang.) | Relative Intensity |
|---|---|---|
| 5.45 | 16.20 | 46 |
| 10.55 | 8.38 | 38 |
| 14.15 | 6.25 | 100 |
| 17.09 | 5.18 | 22 |
| 18.78 | 4.72 | 28 |
| 21.15 | 4.20 | 21 |
| 23.09 | 3.85 | 22 |
| 24.14 | 3.68 | 37 |

TABLE 36
Condensed peak list # 3 for Compound (I) free base Form FB-A-0.

| 2-theta (deg) | d-Spacing (ang.) | Relative Intensity |
|---|---|---|
| 5.45 | 16.20 | 46 |
| 10.55 | 8.38 | 38 |
| 14.15 | 6.25 | 100 |
| 18.78 | 4.72 | 28 |
| 24.14 | 3.68 | 37 |

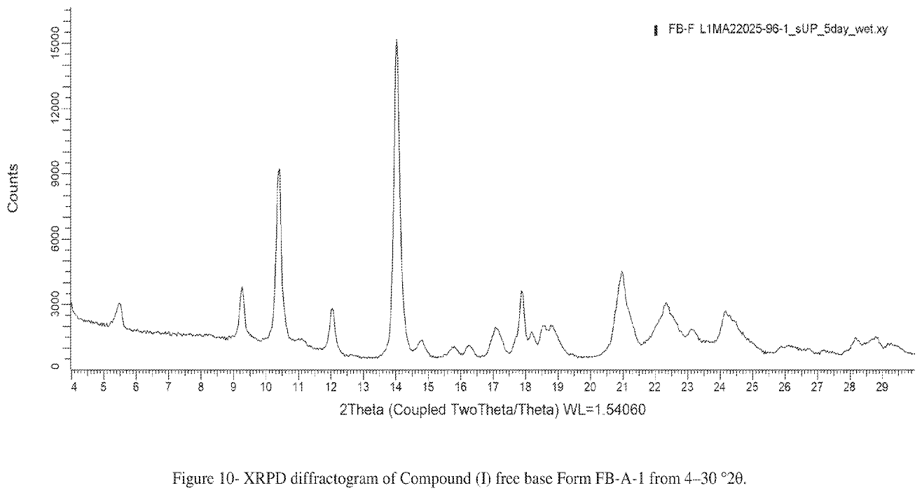

In some embodiments, the present disclosure provides crystalline Form FB-A-1 of free base of Compound (I). The XRPD pattern and peaks are shown in FIG. 10.

TABLE 37
Peak list for Compound (I) free base Form FB-A-1.

| 2-theta (deg) | d-Spacing (ang.) | Relative Intensity |
|---|---|---|
| 5.49 | 16.09 | 8 |
| 9.27 | 9.53 | 17 |
| 10.41 | 8.49 | 56 |
| 11.00 | 8.04 | 3 |
| 12.04 | 7.34 | 14 |
| 14.03 | 6.31 | 100 |
| 14.79 | 5.98 | 5 |
| 15.78 | 5.61 | 3 |
| 16.26 | 5.45 | 3 |
| 17.09 | 5.18 | 9 |
| 17.88 | 4.96 | 20 |
| 18.20 | 4.87 | 8 |
| 18.57 | 4.77 | 10 |
| 18.82 | 4.71 | 9 |
| 20.98 | 4.23 | 26 |
| 22.07 | 4.02 | 9 |
| 22.34 | 3.98 | 16 |
| 23.16 | 3.84 | 7 |
| 24.16 | 3.68 | 13 |
| 24.41 | 3.64 | 10 |
| 28.19 | 3.16 | 5 |
| 28.81 | 3.10 | 5 |

Only peaks with relative intensity 3 or greater reported.

TABLE 38
Condensed peak list # 1 for Compound (I) free base Form FB-A-1.

| 2-theta (deg) | d-Spacing (ang.) | Relative Intensity |
|---|---|---|
| 5.49 | 16.09 | 8 |
| 9.27 | 9.53 | 17 |
| 10.41 | 8.49 | 56 |
| 12.04 | 7.34 | 14 |
| 14.03 | 6.31 | 100 |
| 17.09 | 5.18 | 9 |
| 17.88 | 4.96 | 20 |
| 20.98 | 4.23 | 26 |

TABLE 39
Condensed peak list # 2 for Compound (I) free base Form FB-A-1.

| 2-theta (deg) | d-Spacing (ang.) | Relative Intensity |
|---|---|---|
| 5.49 | 16.09 | 8 |
| 9.27 | 9.53 | 17 |
| 10.41 | 8.49 | 56 |
| 12.04 | 7.34 | 14 |
| 14.03 | 6.31 | 100 |
| 20.98 | 4.23 | 26 |

TABLE 40
Condensed peak list # 3 for Compound (I) free base Form FB-A-1.

| 2-theta (deg) | d-Spacing (ang.) | Relative Intensity |
|---|---|---|
| 9.27 | 9.53 | 17 |
| 10.41 | 8.49 | 56 |
| 12.04 | 7.34 | 14 |
| 14.03 | 6.31 | 100 |

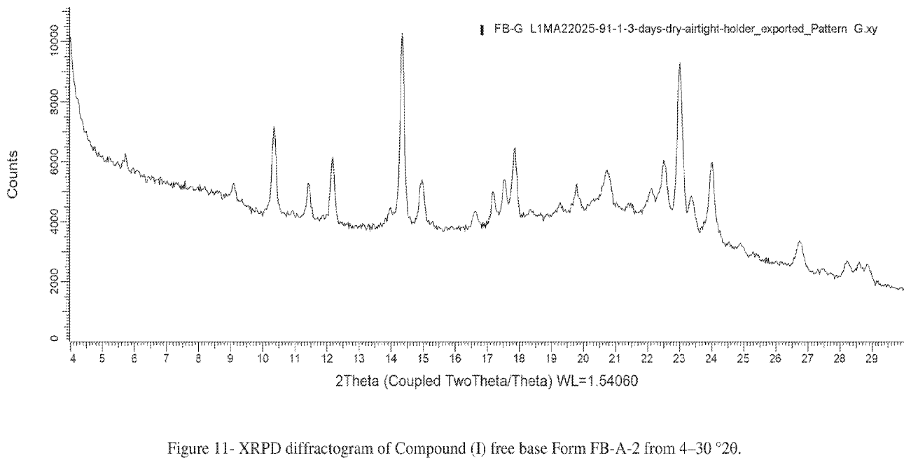

In some embodiments, the present disclosure provides crystalline Form FB-A-2 of free base of Compound (I). The XRPD pattern and peaks are shown in FIG. 11.

TABLE 41
Peak list for Compound (I) free base Form FB-A-2.

| 2-theta (deg) | d-Spacing (ang.) | Relative Intensity |
|---|---|---|
| 9.09 | 9.72 | 11 |
| 10.36 | 8.53 | 45 |
| 11.43 | 7.73 | 18 |
| 12.18 | 7.26 | 34 |
| 13.99 | 6.33 | 11 |
| 14.35 | 6.17 | 100 |
| 14.97 | 5.91 | 25 |
| 16.64 | 5.32 | 9 |
| 17.19 | 5.16 | 20 |
| 17.54 | 5.05 | 26 |
| 17.86 | 4.96 | 42 |
| 18.34 | 4.83 | 12 |
| 19.28 | 4.60 | 16 |
| 19.79 | 4.48 | 26 |
| 20.74 | 4.28 | 35 |
| 21.41 | 4.15 | 19 |
| 22.12 | 4.01 | 28 |
| 22.52 | 3.95 | 44 |
| 23.01 | 3.86 | 95 |
| 23.37 | 3.80 | 28 |
| 24.01 | 3.70 | 46 |
| 24.90 | 3.57 | 7 |
| 26.74 | 3.33 | 15 |
| 28.23 | 3.16 | 9 |
| 28.60 | 3.12 | 9 |
| 28.86 | 3.09 | 9 |

Only peaks with relative intensity 5 or greater reported.

TABLE 42
Condensed peak list #1 for Compound (I) free base Form FB-A-2.

| 2-theta (deg) | d-Spacing (ang.) | Relative Intensity |
|---|---|---|
| 10.36 | 8.53 | 45 |
| 11.43 | 7.73 | 18 |
| 12.18 | 7.26 | 34 |
| 14.35 | 6.17 | 100 |
| 14.97 | 5.91 | 25 |
| 16.64 | 5.32 | 9 |
| 17.19 | 5.16 | 20 |
| 17.54 | 5.05 | 26 |
| 17.86 | 4.96 | 42 |
| 22.52 | 3.95 | 44 |
| 23.01 | 3.86 | 95 |
| 24.01 | 3.70 | 46 |

TABLE 43
Condensed peak list #2 for Compound (I) free base Form FB-A-2.

| 2-theta (deg) | d-Spacing (ang.) | Relative Intensity |
|---|---|---|
| 10.36 | 8.53 | 45 |
| 11.43 | 7.73 | 18 |
| 12.18 | 7.26 | 34 |
| 14.35 | 6.17 | 100 |
| 14.97 | 5.91 | 25 |
| 17.86 | 4.96 | 42 |
| 23.01 | 3.86 | 95 |
| 24.01 | 3.70 | 46 |

TABLE 44
Condensed peak list #3 for Compound (I) free base Form FB-A-2.

| 2-theta (deg) | d-Spacing (ang.) | Relative Intensity |
|---|---|---|
| 10.36 | 8.53 | 45 |
| 12.18 | 7.26 | 34 |
| 14.35 | 6.17 | 100 |
| 23.01 | 3.86 | 95 |
| 24.01 | 3.70 | 46 |

In some embodiments, the present disclosure provides a crystalline form of Compound (I) free base represented by Formula:

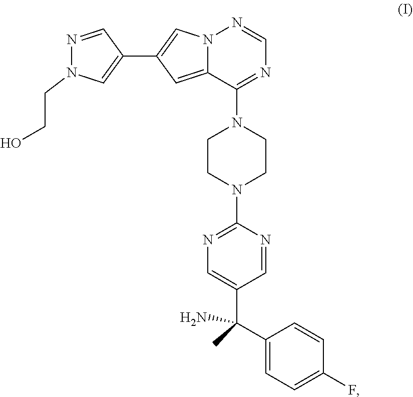 [SMILES: C[C@](N)(c1ccc(F)cc1)c1cnc(N2CCN(c3ncnn4cc(-c5cnn(CCO)c5)cc34)CC2)nc1]

wherein the crystalline form comprises the Form A family of crystalline forms selected from Form FB-A-0, Form FB-A-1 and Form FB-A-2 characterized by at least one of the following:
(a) an X-ray powder diffraction pattern (XRPD) substantially the same as shown in FIGS. 9A, 10, or 11;
(b) an X-ray powder diffraction pattern (XRPD) which comprises at least three peaks chosen from 10.6°, 12.2°, 14.2°, 14.8°, 17.1°, 18.3°, 23.1°, and 24.1;
(c) a thermogravimetric analysis (TGA) substantially similar to FIG. 9B.
(d) a DSC thermogram substantially similar to FIG. 9C.
(e) a DSC thermogram with an endotherm having an onset at 61.6±2° C. and 171.4±2° C.; or
(f) a combination thereof.

### Pharmaceutical Compositions

Some embodiments of the disclosure relate to a pharmaceutical composition comprising a Salt or Solid Form of the disclosure and a pharmaceutically acceptable excipient. Some embodiments of the disclosure relate to a pharmaceutical composition comprising: a pharmaceutically acceptable excipient; and a phosphate salt of Compound (I), wherein the molar ratio between Compound (I) and phosphoric acid is 1:1. In some embodiments, the phosphonate salt is crystalline. In some embodiments, the phosphate salt is in a single crystalline form. In some embodiments, the phosphate salt is unsolvated. In other embodiments, the phosphate salt is solvated. In some embodiments, the phosphate salt of Compound (I) is crystalline Form A. In some embodiments, the phosphate salt of Compound (I) is crystalline Form G. In some embodiments, the phosphate salt of Compound (I) is crystalline Form O.

Some embodiments of the disclosure relate to a pharmaceutical composition comprising: a pharmaceutically acceptable excipient; and a besylate salt of Compound (I), wherein the molar ratio between Compound (I) and benzene sulfonic acid is 1:2. In some embodiments, the besylate salt is crystalline. In some embodiments, the besylate salt is in a single crystalline form. In some embodiments, the besylate salt is unsolvated. In other embodiments, the besylate salt is solvated. In some embodiments, the besylate salt of Compound (I) is crystalline Form 1-A. In some embodiments, the besylate salt of Compound (I) is crystalline Form 1-B.

Some embodiments of the disclosure relate to a pharmaceutical composition comprising: a pharmaceutically acceptable excipient; and a benzoate salt of Compound (I), wherein the molar ratio between Compound (I) and benzoic acid is 1:1. In some embodiments, the benzoate salt is crystalline. In some embodiments, the benzoate salt is in a single crystalline form. In some embodiments, the benzoate salt is unsolvated. In other embodiments, the benzoate salt is solvated. In some embodiments, the benzoate salt of Compound (I) is crystalline Form 2-A. In some embodiments, the benzoate salt of Compound (I) is crystalline Form 2-B.

Some embodiments of the disclosure relate to a pharmaceutical composition comprising: a pharmaceutically acceptable excipient; and a sulfate salt of Compound (I), wherein the molar ratio between Compound (I) and sulfuric acid is 1:1. In some embodiments, the sulfate salt is crystalline. In some embodiments, the sulfate salt is in a single crystalline form. In some embodiments, the sulfate salt is unsolvated. In other embodiments, the sulfate salt is solvated. In some embodiments, the sulfate salt of Compound (I) is crystalline Form 9-A.

Some embodiments of the disclosure relate to a pharmaceutical composition comprising: a pharmaceutically acceptable excipient; and Compound (I) free base. In some embodiments, the free base is in amorphous form. In some embodiments, the free base is crystalline. In some embodiments, the free base is in a single crystalline form. In some embodiments, the free base is unsolvated. In other embodiments, the free base is solvated. In some embodiments, the free base of Compound (I) is crystalline Form FB-A-0. In some embodiments, the free base of Compound (I) is crystalline Form FB-A-1. In some embodiments, the free base of Compound (I) is crystalline Form FB-A-2.

Salts or Solid Forms of the disclosure may be formulated for administration in any convenient way for use in human or veterinary medicine. In some embodiments, the compound or salt included in the pharmaceutical compositions may be active itself, or may be a prodrug, e.g., capable of being converted to an active compound in a physiological setting.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Examples of pharmaceutically acceptable carriers/excipients include: (1) sugars, such as, e.g., lactose, glucose, and sucrose; (2) starches, such as, e.g., corn starch and potato starch; (3) cellulose and its derivatives, such as, e.g., sodium carboxymethyl cellulose, ethyl cellulose, and cellulose acetate; (4) powdered tragacanth; (5) malt; (6) gelatin; (7) talc; (8) excipients, such as, e.g., cocoa butter and suppository waxes; (9) oils, such as, e.g., peanut oil, cottonseed oil, safflower oil, sesame oil, olive oil, corn oil, and soybean oil; (10) glycols, such as, e.g., propylene glycol; (11) polyols, such as, e.g., glycerin, sorbitol, mannitol, and polyethylene glycol; (12) esters, such as, e.g., ethyl oleate and ethyl laurate; (13) agar; (14) buffering agents, such as, e.g., magnesium hydroxide and aluminum hydroxide; (15) alginic acid; (16) pyrogen-free water; (17) isotonic saline; (18) Ringer's solution; (19) ethyl alcohol; (20) phosphate buffer solutions; (21) cyclodextrins, such as, e.g., Captisol®; and (22) other non-toxic compatible substances employed in pharmaceutical formulations.

Examples of pharmaceutically acceptable antioxidants include: (1) water soluble antioxidants, such as, e.g., ascorbic acid, cysteine hydrochloride, sodium bisulfate, sodium metabisulfite, sodium sulfite, and the like; (2) oil-soluble antioxidants, such as, e.g., ascorbyl palmitate, butylated hydroxyanisole (BHA), butylated hydroxytoluene (BHT), lecithin, propyl gallate, alpha-tocopherol, and the like; and (3) metal chelating agents, such as, e.g., citric acid, ethylenediamine tetraacetic acid (EDTA), sorbitol, tartaric acid, phosphoric acid, and the like.

Solid dosage forms (e.g., capsules, tablets, pills, dragees, powders, granules, and the like) can include one or more pharmaceutically acceptable carriers, such as, e.g., sodium citrate or dicalcium phosphate, and/or any of the following: (1) fillers or extenders, such as, e.g., starches, lactose, sucrose, glucose, mannitol, and/or silicic acid; (2) binders, such as, e.g., carboxymethylcellulose, alginates, gelatin, polyvinyl pyrrolidone, sucrose, and/or acacia; (3) humectants, such as, e.g., glycerol; (4) disintegrating agents, such as, e.g., agar-agar, calcium carbonate, potato or tapioca starch, alginic acid, certain silicates, and sodium carbonate; (5) solution retarding agents, such as, e.g., paraffin; (6) absorption accelerators, such as, e.g., quaternary ammonium compounds; (7) wetting agents, such as, e.g., cetyl alcohol and glycerol monostearate; (8) absorbents, such as, e.g., kaolin and bentonite clay; (9) lubricants, such as, e.g., talc, calcium stearate, magnesium stearate, solid polyethylene glycols, sodium lauryl sulfate, and mixtures thereof; and (10) coloring agents.

Liquid dosage forms can include pharmaceutically acceptable emulsions, microemulsions, solutions, suspensions, syrups, and elixirs. In addition to the active ingredient, the liquid dosage forms may contain inert diluents commonly used in the art, such as, e.g., water or other solvents, solubilizing agents, and emulsifiers, such as, e.g., ethyl alcohol, isopropyl alcohol, ethyl carbonate, ethyl acetate, benzyl alcohol, benzyl benzoate, propylene glycol, 1,3-butylene glycol, oils (such as, e.g., cottonseed, groundnut, corn, germ, olive, castor, and sesame oils), glycerol, tetrahydrofuryl alcohol, polyethylene glycols and fatty acid esters of sorbitan, and mixtures thereof.

Suspensions, in addition to the active compound, may contain suspending agents as, e.g., ethoxylated isostearyl alcohols, polyoxyethylene sorbitol and sorbitan esters, microcrystalline cellulose, aluminum metahydroxide, bentonite, agar-agar, tragacanth, and mixtures thereof.

Ointments, pastes, creams and gels may contain, in addition to an active compound, excipients, such as, e.g., animal and vegetable fats, oils, waxes, paraffins, starch, tragacanth, cellulose derivatives, polyethylene glycols, silicones, bentonites, silicic acid, talc, zinc oxide, or mixtures thereof.

Powders and sprays can contain, in addition to an active compound, excipients such as, e.g., lactose, talc, silicic acid, aluminum hydroxide, calcium silicates, and polyamide powder, or mixtures of these substances. Sprays can additionally contain customary propellants, such as, e.g., chlorofluorohydrocarbons and volatile unsubstituted hydrocarbons, such as, e.g., butane and propane.

A Salt or Solid Form of the disclosure can be given per se or as a pharmaceutical composition containing, for example, 0.1 to 99.5% (such as 0.5 to 90%) of active ingredient in combination with a pharmaceutically acceptable carrier.

The formulations can be administered topically, orally, transdermally, rectally, vaginally, parentally, intranasally, intrapulmonary, intraocularly, intravenously, intramuscularly, intraarterially, intrathecally, intracapsularly, intradermally, intraperitoneally, subcutaneously, subcuticularly, or by inhalation.

### Methods of Treatment

Some embodiments of the disclosure relate to methods of treating a patient in need of a KIT or PDGFRα inhibitor by administering a therapeutically effective amount of a Salt or Solid Form of the disclosure.

The Salt and Solid Forms of the disclosure are selective KIT inhibitors. In some embodiments, the Salt and Solid Forms of the disclosure are selective D816V KIT inhibitors. In some embodiments, the Salt and Solid Forms of the disclosure are selective PDGFRα inhibitors. In some embodiments, the Salt and Solid Forms of the disclosure are selective PDGFRα exon 18 inhibitors. In some embodiments, the Salt and Solid Forms of the disclosure are selective PDGFRα D842V inhibitors. As used herein, a “selective KIT inhibitor” or a “selective PDGFRα inhibitor” refers to a Salt or Solid Form of the disclosure that selectively inhibits a KIT protein kinase or PDGFRα protein kinase over another protein kinase and exhibits at least a 2-fold selectivity for a KIT protein kinase or a PDGFRα protein kinase over another kinase. For example, a selective KIT inhibitor or a selective PDGFRA inhibitor exhibits at least a 9-fold selectivity, 10-fold selectivity; at least a 15-fold selectivity; at least a 20-fold selectivity; at least a 30-fold selectivity; at least a 40-fold selectivity; at least a 50-fold selectivity; at least a 60-fold selectivity; at least a 70-fold selectivity; at least a 80-fold selectivity; at least a 90-fold selectivity; at least 100-fold, at least 125-fold, at least 150-fold, at least 175-fold, or at least 200-fold selectivity for a KIT protein kinase or a PDGFRα kinase over another kinase. In some embodiments, a selective KIT inhibitor or a selective PDGFRα inhibitor exhibits at least 150-fold selectivity over another kinase, e.g., VEGFR2 (vascular endothelial growth factor receptor 2), SRC (Non-receptor protein tyrosine kinase), and FLT3 (Fms-Like Tyrosine kinase 3). In some embodiments, a selective KIT or a selective PDGFRα inhibitor exhibits selectivity over PDGRFβ, CSF1R (colony stimulating factor receptor 1), and FLT3.

In some embodiments, a selective KIT or a selective PDGFRα inhibitor exhibits selective over LCK (lymphocyte-specific protein kinase), ABL (nuclear protein tyrosine kinase), never-in-mitosis gene A (NIMA)-related kinase 5 (NEK5), and ROCK1 (rho-associated coil-coil-continuing protein kinase-1). In some embodiments, selectivity for a KIT protein kinase or a PDGFRα protein kinase over another kinase is measured in a cellular assay (e.g., a cellular assay). In some embodiments, selectivity for a KIT protein kinase or a PDGFRα protein kinase over another kinase is measured in a biochemical assay (e.g., a biochemical assay).

The Salt and Solid Forms of the disclosure are selective over ion channels. In some embodiments, a selective KIT or a selective PDGFRα inhibitor has limited potential to inhibit human voltage-gated sodium channel (hNav 1.2).

The Salt and Solid Forms of the disclosure are selective for mutant KIT over wild type KIT. In some embodiments, the Salt and Solid Forms of the disclosure are selective for exon 17 mutant KIT over wild type KIT.

The Salt and Solid Forms of the disclosure can be useful for treating diseases or conditions associated with mutant KIT or mutant PDGFRA activity in humans or non-humans. In some embodiments, the Salt and Solid Forms of the disclosure are for use as a medicament. In some embodiments, the Salt and Solid Forms of the disclosure are for use in therapy. In some embodiments, the Salt and Solid Forms of the disclosure are for use in the manufacture of a medicament. In some embodiments, the disclosure provides methods for treating KIT-driven malignancies, include mastocytosis (SM), GIST (gastrointestinal stromal tumors), AML (acute myeloid leukemia), melanoma, seminoma, intracranial germ cell tumors, and/or mediastinal B-cell lymphoma. In addition, mutations in KIT have been linked to Ewing's sarcoma, DLBCL (diffuse large B cell lymphoma), dysgerminoma, MDS (myelodysplastic syndrome), NKTCL (nasal NK/T-cell lymphoma), CMML (chronic myelomonocytic leukemia), and brain cancers. In some embodiments, the disclosure provides methods for treating Ewing's sarcoma, DLBCL, dysgerminoma, MDS, NKTCL, CMML, and/or brain cancers. KIT mutations have also been found in thyroid cancer, colorectal cancer, endometrial cancer, bladder cancer, NSCLC, and breast cancer (AACR Project GENIE). In some embodiments, Salt and Solid Forms of the disclosure can be useful for treating mast cell activation syndrome (MCAS). Salt and Solid Forms of the disclosure can be useful for treating systemic mastocytosis. Salt and Solid Forms of the disclosure can be useful for treating advanced systemic mastocytosis. Salt and Solid Forms of the disclosure can be useful for treating indolent SM and smoldering SM. Salt and Solid Forms of the disclosure can be useful for treating GIST.

Salt and Solid Forms of the disclosure can be useful for treating diseases or conditions associated with the KIT mutations in Exon 9, Exon 11, Exon 14, Exon 17, and/or Exon 18 of the KIT gene sequence. Salt and Solid Forms of the disclosure can be useful for treating diseases or conditions associated with PDGFRA mutations in Exon 12, Exon 14, and/or Exon 18 of the PDGFRA gene sequence. In some embodiments, provided herein are methods for treating a disease or condition associated with at least one KIT mutation in Exon 9, Exon 11, Exon 14, Exon 17, and/or Exon 18 of the KIT gene sequence. In some embodiments, methods for treating a disease or condition associated with at least one PDGFRA mutation in Exon 12, Exon 14, and/or Exon 18 of the PDGFRA gene sequence are provided.

Salt and Solid Forms of the disclosure can be active against one or more KIT protein kinases with mutations in Exon 17 of the KIT gene sequence (e.g., KIT protein mutations D816V, D816Y, D816F, D816K, D816H, D816A, D816G, D816E, D816I, D816F, D820A, D820E, D820G, D820Y, N822K, N822H, V560G, Y823D, and A829P), and much less active against wild-type KIT protein kinase. In some embodiments, provided herein are methods for treating a disease or condition associated with at least one KIT mutation such as those chosen from D816V, D816Y, D816F, D816K, D816H, D816A, D816G, D816E, D816I, D816F, D820A, D820E, D820G, D820Y, N822K, N822H, V560G, Y823D, and A829P. In some embodiments, provided herein are methods for treating a disease or condition associated with at least one KIT mutation such as, e.g., those chosen from C809, C809G, D816H, D820A, D820G, N822H, N822K, and Y823D.

Salt and Solid Forms of the disclosure can be active against one or more KIT protein kinases with mutations in Exon 11 of the KIT gene sequence (e.g., KIT protein mutations del557-559insF, V559G/D). In some embodiments, provided herein are methods for treating a disease or condition associated with at least one KIT mutation, such as, e.g., those chosen from L576P, V559D, V560D, V560G, W557G, Del 554-558EVQWK, del557-559insF, Del EVQWK554-558, Del EVQWKVVEEINGNNYVYI554-571, Del KPMYEVQWK550-558, Del KPMYEVQW550-557FL, Del KV558-559, Del KV558-559N, Del MYEVQW552-557, Del PMYE551-554, Del VV559-560, Del WKVVE557-561, Del WK557-558, Del WKVV557-560C, Del WKVV557-560F, DelYEVQWK553-558, and insertion K558NP.

Salt and Solid Forms of the disclosure can be active against one or more KIT protein kinases with mutations in Exon 11/13 of the KIT gene sequence (e.g., KIT protein mutations V559D/V654A, V560G/D816V, and V560G/822K). In some embodiments, provided here are methods for treating a disease or condition associated with one or more KIT mutations in Exon 11/13).

Salt and Solid Forms of the disclosure can be active against one or more KIT protein kinases with mutations in Exon 9 of the KIT gene sequence. In some embodiments, provided herein are methods for treating a disease or condition associated with at least one KIT mutation in Exon 9.

In some embodiments, Salt and Solid Forms of the disclosure are not active against KIT protein kinases with the mutations V654A, N655T, T670I, and/or N680.

Salt and Solid Forms of the disclosure can be active against one or more PDGFRα protein kinases with mutations. In some embodiments, provided herein are methods for treating a disease or condition associated with at least one PDGFRA mutation in Exon 12 of the PDGFRA gene sequence, such as, e.g., PDGFRα protein mutations V561D, Del RV560-561, Del RVIES560-564, Ins ER561-562, SPDGHE566-571R, SPDGHE566-571K, or Ins YDSRW582-586. In some embodiments, provided herein are methods for treating a disease or condition associated with at least one PDGFRA mutation in Exon 14 of the PDGFRA gene sequence, such as, e.g., PDGFRα protein mutation N659K. In some embodiments, provided herein are methods for treating a disease or condition associated with at least one PDGFRA mutation in Exon 18 of the PDGFRA gene sequence, such as, e.g., PDGFRα protein mutations D842V, D842Y, D842I, DI842-843IM, D846Y, Y849C, Del D842, Del I843, Del RD841-842, Del DIM842-845, Del DIMH842-845, Del IMHD843-846, Del MHDS844-847, RD841-842KI, DIMH842-845A, DIMH842-845V, DIMHD842-846E, DIMHD842-846S, DIMHD842-846N, DIMHD842-846G, IMHDS843-847T, IMHDS8843-847M, or HDSN845-848P.

Salt and Solid Forms of the disclosure can be active against one or more PDGFRα protein kinases with mutations Exon 18 in the PDGFRA gene sequence (e.g., protein mutations PDGFRα D842V, PDGFRα D842I, or PDGFRα D842Y). In some embodiments, provided herein are methods for treating a disease or condition associated with at least one PDGFRA mutation in Exon 18, such as, e.g., protein mutation PDGFR& D842V.

Salt and Solid Forms of the disclosure can be useful for treating an eosinophilic disorder. In some embodiments, the eosinophilic disorder is mediated by mutant KIT or PDGFRα. In some embodiments, that eosinophilic disorder is mediated by wild type KIT or PDGFRα. In some embodiments, provided herein are methods for treating an eosinophilic disorder, comprising administering to a subject a therapeutically effective amount of the Salt and Solid Forms of the disclosure or a pharmaceutically acceptable salt thereof and/or solvate of any of the foregoing. In one embodiment, the eosinophilic disorder is selected from hypereosinophilic syndrome, eosinophilia, eosinophilic enterogastritis, eosinophilic leukemia, eosinophilic granuloma and Kimura's disease.

In some embodiments, eosinophilic disorder is selected from hypereosinophilic syndrome, eosinophilia, eosinophilic enterogastritis, eosinophilic leukemia, eosinophilic granuloma and Kimura's disease. Other eosinophilic disorders include eosinophilic esophagitis, eosinophilic gastroenteritis, eosinophilic fasciitis, and Churg-Strauss syndrome.

In one embodiment, the eosinophilic disorder is hypereosinophilic syndrome. In a specific embodiment, the hypereosinophilic syndrome is idiopathic hypereosinophilic syndrome. In one embodiment, the eosinophilic disorder is eosinophilic leukemia. In a specific embodiment, the eosinophilic leukemia is chronic eosinophilic leukemia. In another embodiment, the eosinophilic disorder is refractory to treatment with imatinib, sunitinib, and/or regorafenib. In a specific embodiment, the eosinophilic disorder is refractory to treatment with imatinib.

Salt and Solid Forms of the disclosure can be useful for reducing the number of eosinophils in a subject in need thereof. In some embodiments, provided herein are methods for reducing the number of eosinophils in a subject in need thereof comprising administering to the subject a therapeutically effective amount of a Salt and Solid Forms of the disclosure or a pharmaceutically acceptable salt thereof and/or a solvate of any of the foregoing.

In one embodiment, the disclosed methods reduce the number of eosinophils in the blood, bone marrow, gastrointestinal tract (e.g., esophagus, stomach, small intestine and colon), or lung. In another embodiment, a method disclosed herein reduces the number of blood eosinophils. In a further embodiment, a method disclosed herein reduces the number of lung eosinophils. In still a further embodiment, a method disclosed herein reduces the number of eosinophil precursor cells.

In another embodiment, the disclosed methods reduce (post-administration) the number of eosinophils by at least about 10%, at least about 20%, at least about 30%, at least about 40%, at least about 50%, at least about 60%, at least about 70%, at least about 80%; at least about 90%, at least about 95% or at least about 99%. In a specific embodiment, a method disclosed herein reduces the number of eosinophils below the limit of detection.

In another embodiment, the disclosed methods reduce (post-administration) the number of eosinophil precursors by at least about 10%, at least about 20%, at least about 30%, at least about 40%, at least about 50%, at least about 60%, at least about 70%, at least about 80%, at least about 90%, at least about 95% or at least about 99%. In a specific embodiment, a method disclosed herein reduces the number of eosinophil precursors below the limit of detection.

Salt and Solid Forms of the disclosure can be useful for treating mast cell disorders. Salt and Solid Forms of the disclosure can be useful for treating mastocytosis. Mastocytosis is subdivided into two groups of disorders: (1) cutaneous mastocytosis (CM) describes forms that are limited to the skin; and (2) systemic mastocytosis (SM) describes forms in which mast cells infiltrate extracutaneous organs, with or without skin involvement. SM is further subdivided into five forms: indolent (ISM); smoldering (SSM); aggressive (ASM); SM with associated hemotologic non-mast cell lineage disease (SM-AHNMD); and mast cell leukemia (MCL).

Diagnosis of SM is based in part on histological and cytological studies of bone marrow showing infiltration by mast cells of often atypical morphology, which frequently abnormally express non-mast cell markers (CD25 and/or CD2). Diagnosis of SM is confirmed when bone marrow mast cell infiltration occurs in the context of one of the following: (1) abnormal mast cell morphology (spindle-shaped cells); (2) elevated level of serum tryptase above 20 ng/mL; or (3) the presence of the activating KIT protein mutations, such as, e.g., exon 17 mutations such as D816 mutations such as D816V.

Activating mutations at the D816 position are found in the vast majority of mastocytosis cases (90-98%), with the most common mutations being D816V, D816H, and D816Y. The D816V mutation is found in the activation loop of the protein kinase domain and leads to constitutive activation of KIT kinase.

No drugs are approved for the non-advanced forms of systemic mastocytosis, ISM or SSM. Current approaches to management of these chronic diseases include nonspecific symptom-directed therapies that have varying degrees of efficacy and no effect on MC burden. Cytoreductive therapies, such as cladribine and interferon alpha, are occasionally used for intractable symptoms. Based on the current treatment landscape, there remains an unmet medical need in patients with ISM and SSM with moderate-to-severe symptoms that cannot be adequately managed by available symptom-directed therapies.

Salt and Solid Forms of the disclosure can be useful for treating ISM or SSM. In some embodiments, the patient with ISM or SSM has symptoms that are inadequately controlled by at least one, at least two, at least three symptomatic treatments. Symptoms can be assessed using a patient reported outcome (PRO) tool e.g. the Indolent Systemic Mastocytosis-Symptom Assessment Form (ISM-SAF) (ISPOR Europe 2019, Copenhagen Denmark, 2-6 Nov. 2019). Salt and Solid Forms of the disclosure can be useful for improving symptoms associated with ISM or SSM e.g., reducing or eliminating pruritus, flushing, headaches, and/or GI events, such as vomiting, diarrhea, and abdominal pain. Improvements in symptoms can be assessed using the ISM-SAF.

Salt and Solid Forms of the disclosure can be useful for treating other mast cell disorders, such as mast cell activation syndrome (MCAS), and hereditary alpha tryptasemia (HAT) (Picard Clin. Ther. 2013, May 35(5) 548; Akin J. Allergy Clin. Immuno. 140(2) 349 62. Salt and Solid Forms of the disclosure can be useful for treating mast cell disorders associated with KIT and PDGFRα mutations. Salt and Solid Forms of the disclosure can be useful for treating mast cell diseases associated with wild type KIT and PDGFRα.

Salt and Solid Forms of the disclosure can be useful for treating mast cell activation syndrome (MCAS), which is an immunological condition in which mast cells inappropriately and excessively release chemical mediators, resulting in a range of chronic symptoms, sometimes including anaphylaxis or near-anaphylaxis attacks. Unlike mastocytosis, where patients have an abnormally increased number of mast cells, patients with MCAS have a normal number of mast cells that do not function properly and are defined as “hyperresponsive.” Types of MCAS include primary MCAS (monoclonal mast cell activation syndrome (MMAS)), secondary MCAS (MCAS that arises from another disease), and idiopathic MCAS (MCAS that rules out primary or secondary MCAS).

Disclosed herein are improved methods for treating indolent systemic mastocytosis (ISM) and monoclonal mast cell activation syndrome in patients with Compound (I) or a pharmaceutically acceptable salt thereof (e.g., Salt and Solid Forms of the disclosure). The disclosure provides dosing regimens of Compound (I) for the treatment of ISM and mMCAS. More specifically, the disclosure provides methods for treating ISM and mMCAS in patients by administering Compound (I) once daily dose in an amount of 15 mg to 200 mg. An object of this disclosure is to provide new methods of treating ISM and mMCAS with a safe and effective once daily dose.

Non-limiting embodiments of the present disclosure include:

Embodiment 1. A method of treating indolent systemic mastocytosis (ISM) or monoclonal mast cell activation syndrome (mMCAS) comprising orally administering to a patient in need thereof an amount of 15 mg to 200 mg of Compound (I)

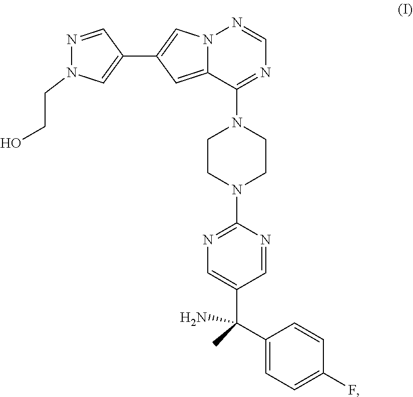 [SMILES: C[C@](N)(c1ccc(F)cc1)c1cnc(N2CCN(c3ncnn4cc(-c5cnn(CCO)c5)cc34)CC2)nc1]

or a pharmaceutically acceptable salt thereof in an amount equivalent to 15 mg to 200 mg of Compound (I), once a day.

Embodiment 2. The method of embodiment 1, wherein the patient has ISM.

Embodiment 3. The method of embodiment 1 or 2, wherein the patient has moderate to servere ISM.

Embodiment 4. The method of embodiment 3, wherein the patient has a Total Symptom Score (TSS)≥28 of the Indolent Systemic Mastocytosis-Symptom Assessment Form (ISM-SAF) at Baseline. In an alternate embodiment, the patient has a TSS<28 of the ISM-SAF at Baseline.

Embodiment 5. The method of any embodiments 1-4, wherein the patient has ≥1 symptom in skin or GI domains of the Indolent Systemic Mastocytosis-Symptom Assessment Form (ISM-SAF) at Baseline.

Embodiment 6. The method of embodiment 1, wherein the patient has mMCAS.

Embodiment 7. The method of any one of embodiments 1-6, wherein the amount is 25 to 100 mg.

Embodiment 8. The method of embodiment 7, wherein the amount is 100 mg.

Embodiment 9. The method of embodiment 7, wherein the amount is 50 mg.

Embodiment 10. The method of embodiment 7, wherein the amount is 25 mg.

Embodiment 11. The method of any one of embodiments 1-10, wherein Compound (I) is administered as the free base.

Embodiment 12. The method of any one of embodiments 1-10, wherein a Compound (I) is administered as the pharmaceutically acceptable salt.

Embodiment 13. The method of embodiment 12, wherein the pharmaceutically acceptable salt of Compound (I) is the salt. In some aspects, the salt is the phosphate salt. In some aspects, the salt is the besylate salt. In some aspects, the salt is the benzoic acid salt. In some aspects, the salt is the sulfate.

Embodiment 14. The method of any one of embodiments 1-13, wherein treating results in a reduction of mast cell burden.

Embodiment 15. The method of any one of embodiments 1-14, wherein treating results in one or more systemic mastocytosis symptoms.

Embodiment 16. The method of any one of embodiments 1-15, wherein treating results in a reduction in analphylatic episodes.

Embodiment 17. The method of any one of embodiments 1-16, wherein treating results in an improves quality of life (QoL) as measured by one or more questionnaires.

Embodiment 18. The method of any one of embodiments 1-17, wherein treating results in a reduction in the patient's TSS score compared to the patient's TSS Baseline score as assessed by the ISM-SAF.

In some embodiments, the patient is administered Compound (I) as free base (including various solid forms of the free base disclosed herein). In some embodiments, the patient is administered Compound (I) as a pharmaceutically acceptable salt (including various solid forms of the pharmaceutically acceptable salt disclosed herein). In some embodiments, the pharmaceutically acceptable salt is phosphate.

In some embodiments, treating a patient with Compound (I) or a pharmaceutically acceptable salt thereof in an amount disclosed herein comprises a reduction of mast cell burden. In some embodiments, objective measures of mast cell burden include serum tryptase levels, bone marrow mast cell numbers, skin mast cell infiltrates, and KIT D816V mutant allele burden in blood. In some embodiments, objective measures of mast cell burden include serum tryptase, bone marrow mast cell numbers, and KIT D816V mutant allele burden in blood. In some embodiments, treating a patient with an amount of 50 mg of Compound (I) or with an equivalent amount of a pharmaceutically acceptable salt thereof decreases the patient's serum tryptase levels. In some embodiments, treating a patient with an amount of 100 mg of Compound (I) or with an equivalent amount of a pharmaceutically acceptable salt thereof decreases the patient's serum tryptase levels.

In some embodiments, treating a patient with Compound (I) or a pharmaceutically acceptable salt thereof in an amount disclosed herein comprises a reduction of one or more systemic mastocytosis symptoms. Systemic mastocytosis symptoms include, but are not limited to, pruritus, flushing, GI cramping, diarrhea, anaphylaxis (especially to bee venom), bone pain, osteoporosis, and urticarial pigmentosa. In some embodiments, an ISM-SAF (self-assessment form) patient reported outcome (PRO) instrument as defined herein is used to assess symptom improvement. In some embodiments, the patient completes the ISM-SAF once a day prior to receiving treatment and the patient also completes the ISM-SAF once a day while on treatment. For example, the patient completes the ISM-SAF for a period of time, e.g., four weeks, beginning at the time of informed consent, during which time best-supportive care (BSC) medications are optimized and stabilized. Once data from the period of time, e.g., four weeks, are collected, the ISM-SAF is completed once a day for an additional period of time, e.g., two weeks (14 days), and patient eligibility is determined based on the ISM-SAF symptom threshold. Patients meeting the ISM-SAF threshold for eligibility then complete the ISM-SAF once a day while screening procedures are completed to assess study eligibility.

Once all screening procedures are completed, baseline symptoms are collected for a period of time, e.g., 14 days, immediately preceding study entry. These data are used as a Baseline TSS. The ISM-SAF is completed by the patient once a day through completion the study. In some embodiments, the primary endpoint for the study is mean change in ISM-SAF TSS from Baseline. In some embodiments, treating with Compound (I) or a pharmaceutically acceptable salt thereof reduces the number of episodes of anaphylaxis. In some embodiments, an “episode of anaphalaxis” is an episode of anaphylaxis treated with epinephrine.

In some embodiments, treating a patient with Compound (I) or a pharmaceutically acceptable salt thereof in an amount disclosed herein improves quality of life (QoL) as measured by one or more questionnaires. Non-limiting examples of QoL questionnaires include the MC-QoL, the PGIS, the SF-12, the PGIC, and the EQ-5D-EL. The MC-QoL is a disease-specific QoL tool developed specifically for use in patients with ISM and CM (Siebenhaar, F. et al., Allergy 71(6):869-77 (2016)). The MC-QoL contains 27 items assessing four domains: symptoms, emotions, social life/functioning, and skin. Items are assessed on a 5-point scale with a recall period of two weeks. The PGIS is a single-item scale that assesses a patient's perception of disease symptoms at a point in time. The PGIS has been widely used to evaluate a patient's overall sense of whether a treatment has been beneficial. The SF-12 was developed for the Medical Outcomes Study, a multiyear study of patients with chronic conditions. The instrument was designed to reduce respondent burden, while achieving minimum standards of precision for purposes of group comparisons involving multiple health dimensions. The questionnaire measures health and wellbeing using 8 health domains from the patient's perspective. The recall period is four weeks. The PGIC is a single-item scale that assesses a patient's perception of change in disease symptoms at a point in time. The EQ-5D-5L is a standardized instrument for measuring generic health status. It is made up of two components: health state description and evaluation. Health status is measured in terms of five dimensions (5D): mobility; self-care; usual activities; pain/discomfort; and anxiety/depression. Respondents self-rate their level of severity for each dimension using a 5-point scale. The recall period is “today” (Whynes, D. K., Health Qual Life Outcomes 6:94 (2008)).

In some embodiments, treating a patient with Compound (I) or a pharmaceutically acceptable salt thereof in an amount disclosed herein improves bone density. Bone density is measured by a dual-energy x-ray absorptiometry scan assessing both lumbar spine and hip. In some embodiments, treatment does not affect bone density.

As used herein, “SD” means stable disease.

As used herein, “CR” means complete response.

As used herein, “PFS” means progression free survival.

As used herein, the “Indolent Systemic Mastocytosis-Symptom Assessment Form” (©Blueprint Medicines Corporation) or “ISM-SAF” (ISPOR Europe 2019, Copenhagen Denmark, 2-6 Nov. 2019) is employed for the daily patient reported outcome (PRO) assessment on e.g., an eDiary. The ISM-SAF is a 12-item PRO developed specifically to assess symptoms in patients with ISM and SSM. Though primarily developed for evaluating treatment efficacy hypotheses, the ISM-SAF can also be used to screen participants into (or out of) clinical studies based on a minimum level of sign and symptom severity. Eleven items shown in the table below are graded on a 10-point scale (0 to 10, none to maximum severity), and 1 item (diarrhea) also assesses frequency.

| Item | Symptom |
|---|---|
| 1 | Bone pain |
| 2 | Abdominal pain |
| 3 | Nausea |
| 4 | Spots |
| 5 | Itching |
| 6 | Flushing |
| 7 | Fatigue |
| 8 | Dizziness |
| 9 | Brain Fog |
| 10 | Headache |
| 11 | Diarrhea frequency |
| 12 | Diarrhea severity |

The ISM-SAF generates scores for each item, for the domains of skin/Skin Symptom Score (SSS), GI/Gastrointestinal Symptom Score (GSS), and nonspecific symptoms, and a Total Symptom Score (TSS). The TSS is the addition of all symptoms together. In one aspect, TSS is items 1-10 and 12. In one aspect, GSS is items 2-3 and 12. In one aspect, SSS is items 4-6. In one aspect, the patient completes the ISM-SAF daily for 4 weeks beginning at the time of informed consent, during which time BSC medications are optimized and stabilized. Once 4 weeks of data have been collected, the ISM-SAF is completed daily for an additional 2 weeks (14 days) to determine patient eligibility based on the ISM-SAF symptom threshold. Patients meeting the ISM-SAF threshold for eligibility complete the ISM-SAF daily while screening procedures are completed to assess study eligibility. Once all screening procedures are completed, Baseline symptoms are collected for the 14 days immediately preceding study entry. These data will be used as a Baseline TSS.

In one aspect, the patient with ISM has moderate-to-severe symptoms characterized by a minimum TSS. In one aspect, the patient with ISM has moderate-to-severe symptoms characterized by a minimum TSS of ≥28 as assessed using the ISM-SAF. In one aspect, the patient with ISM or SSM with moderate-to-severe symptoms has a minimum TSS of ≥28 and >1 symptom in skin or GI domains of the ISM-SAF at baseline. In one aspect, the patient with ISM has a TSS<28. In one aspect, baseline is the 14-day period before cycle 1 day 1 (C1D1). In one aspect, the patient is not experiencing an acute flare of symptoms beyond their typical baseline symptoms. In one aspect, the patient has failed to achieve symptom control for 1 or more baseline symptoms, as determined by the investigator, with at least 2 of the following symptomatic therapies administered at optimal (approved) dose and for a minimum of 4 weeks (28 days) before starting the ISM-SAF for determination of eligibility: H1 blockers, H2 blockers, proton-pump inhibitors, leukotriene inhibitors, cromolyn sodium, corticosteroids, or omalizumab. In one aspect, the patient has a baseline serum tryptase of <20 ng/mL. In one aspect, the patient has a baseline serum tryptase of ≥20 ng/mL.

In one aspect, treating a patient with Compound (I) or a pharmaceutically acceptable salt thereof results in a reduction of TSS score in comparison the patient's TSS Baseline score as assessed by ISM-SAF. In one aspect, the patient's TSS reduces by 5, 6, 7, 8, 9, 10, 11, 12, 13, 14, 15, 16, 17, 18, 19, 20, 21, 22, 23.

In one aspect, the patient with ISM has KIT D816V mutation. The KIT D816V mutation can be detected by a high sensitivity assay such as a droplet digital polymerase chain reaction (ddPCR) assay with a limit of detection (LOD) of 0.022% mutant allele frequency (MAF).

As used herein “BSC” means best supportive care. More specifically, examples of best supportive care medications include:

| Drug Class | Generic/Trade Name |
|---|---|
| H1 antihistamines | Loratidine/Claritin |
| | Diphenhydramine/Benadryl |
| | Cetirizine/Zyrtec |
| | Fexofenadine/Allegra |
| | Hydroxyzine/Vistaril/Atarax |
| | Rupatadine/Rupafin |
| H2 antihistamines | Cimetidine/Tagamet |
| | Famotidine/Pepcid |
| | Ranitidine/Zantac |
| Proton-pump inhibitors | Omeprazole/Prilosec |
| | Pantoprazole/Protonix |
| | Rabeprazole/AcipHex |
| Leukotriene inhibitors | Montelukast/Singulair |
| | Zafirlukast/Accolate |
| Corticosteroids | Prednisone/Deltasone |
| Cromolyn sodium | Cromoglicic |
| | acid/Nasalcrom/Gastrocrom |
| Anti-IgE antibody | Omalizumab/Xolair |
| Bisphosphonates for osteoporosis | Alendronate/Aledronic |
| | acid//Fosamax |
| | Risedronate/Risedronic |
| | acid/Actenol/Atelvia |
| | Ibandronate/Ibandronic |
| | acid/Boniva |
| | Pamidronic acid/Aredia |
| | Zoledronic acid/Reclast/Zometa |
| Other drugs for osteoporosis | Denosumab/Prolia |
| | Raloxifene/Evista |
| | Teriparatide/Forteo |
| Epinephrine for allergic reactions | Adrenalin/EpiPen |

In some embodiment, the patient treated with Compound (I) or a pharmaceutically acceptable salt disclosed herein is co-administered a proton pump inhibitor. In some embodiments, the patient is treated with Compound (I) or a pharmaceutically acceptable salt herein and is co-administered a proton pump inhibitor under fasted conditions. In some embodiments, the patient is treated with Compound (I) or a pharmaceutically acceptable salt herein and is co-administered a proton pump inhibitor following a moderate-fat meal. In some embodiments, the patient is treated with Compound (I) or a pharmaceutically acceptable salt herein and is co-administered a proton pump inhibitor following a high-fat meal.

As used herein, an “adverse event” or “AE” is any untoward medical occurrence associated with the use of a drug in humans, regardless of whether it is considered drug-related. An AE (also referred to as an adverse experience) can be any unfavorable and unintended sign (e.g., an abnormal laboratory finding), symptom, or disease temporally associated with the use of a drug, without any judgement about causality. An AE can arise from any use of the drug (e.g., off-label use, use in combination with another drug) and from any route of administration, formulation, or dose, including an overdose.

As used herein, the terms “about” and “approximately,” when used in connection with doses, amounts, or weight percent of ingredients of a composition or a dosage form, include the value of a specified dose, amount, or weight percent or a range of the dose, amount, or weight percent that is recognized by one of ordinary skill in the art to provide a pharmacological effect equivalent to that obtained from the specified dose, amount, or weight percent.

The disclosure provides a method of treating indolent systemic mastocytosis comprising administering to a patient in need thereof an amount of 15 mg to 200 mg of Compound (I) or a pharmaceutically acceptable salt thereof in an amount equivalent to 55 mg to 200 mg of Compound (I), once a day. In some embodiments, the patient in need thereof is administered an amount of 10 mg to 200 mg of Compound (I) once a day. In some embodiments, the patient in need thereof is administered an amount of 15 mg, 25 mg, 50 mg, 100 mg, 125 mg, 150 mg, 175 mg, or 200 mg of Compound (I) (or a pharmaceutically acceptable salt thereof in an amount equivalent to 15 mg, 25 mg, 50 mg, 100 mg, 125 mg, 150 mg, 175 mg or 200 mg of Compound (I)) once a day. In some embodiments, the amount is from 50 mg to 200 mg once a day. In some embodiments, the amount is from 100 mg to 200 mg once a day. In some embodiments, the amount is from 50 mg to 100 mg once a day.

Salt and Solid Forms of the disclosure can be useful for treating hereditary alpha tryptasemia (HAT)(overexpression of TPSAB1 causing elevated tryptase)).

Other mast cell diseases include mast cell mediated asthma, anaphylaxis (including idiopathic, Ig-E and non-Ig-E mediated), urticaria (including idiopathic and chronic), atopic dermatitis, swelling (angioedema), irritable bowel syndrome, mastocytic gastroenteritis, mastocytic colitis, pruritus, chronic pruritis, pruritis secondary to chronic kidney failure and heart, vascular, intestinal, brain, kidney, liver, pancreas, muscle, bone and skin conditions associated with mast cells. In some embodiments, the mast cell disease is not associated with mutant KIT or mutant PDGFRα.

KIT and PDGFRA mutations have been extensively studied in GIST. Salt and Solid Forms of the disclosure can be useful for treating GIST associated with KIT mutations. Salt and Solid Forms of the disclosure can be useful for treating unresectable or metastatic GIST. Nearly 80% of metastatic GISTs have a primary activating mutation in either the extracellular region (exon 9) or the juxtamembrane (JM) domain (exon 11) of the KIT gene sequence. Many mutant KIT tumors respond to treatment with targeted therapy such as imatinib, a selective tyrosine kinase inhibitor that specifically inhibits BCR-ABL, KIT, and PDGFRA proteins. However, most GIST patients eventually relapse due to a secondary mutation in KIT that markedly decreases the binding affinity of imatinib. These resistance mutations invariably arise within the adenosine 5-triphosphate (ATP)-binding pocket (exons 13 and 14) or the activation loop (exons 17 and 18) of the kinase gene. Of the currently approved agents for GIST, none are selective targeted agents. Imatinib is currently approved for the treatment of GIST; multikinase inhibitors are used after imatinib. In many cases, these multikinase inhibitors, such as, e.g., sunitinib, regorafenib, and midostaurin, only weakly inhibit imatinib resistant mutants and/or the multikinase inhibitors are limited by a more complex safety profile and a small therapeutic window. In some embodiments, Salt and Solid Forms of the disclosure can be useful for treating GIST in patients who have been treated with imatinib. Salt and Solid Forms of the Disclosure can be useful for treating GIST as first line (1L), second line (2L), third line (3L) or fourth line (4L) therapy.

Salt and Solid Forms of the disclosure can be useful for treating GIST when particular mutations in KIT are absent or present. In some embodiments, Salt and Solid Forms of the disclosure are capable of treating GIST when particular mutations in KIT are absent. In certain embodiments, Salt and Solid Forms of the disclosure are not capable of treating GIST when particular mutations in KIT are present. In some embodiments, Salt and Solid Forms of the disclosure do not provide clinical benefit in patients harboring KIT ATP binding pocket mutations (KIT protein mutations V654A, N655T, and/or T670I).

Salt and Solid Forms of the disclosure can be useful for treating GIST associated with PDGFRA mutations. In 5 to 6% of unresectable of metastatic GIST patients, an activation loop mutation in exon 18 of the gene sequence of PDGFRA at the protein amino acid 842 occurs as the primary mutation.

Salt and Solid Forms of the disclosure can also be useful in treating AML. AML patients also harbor KIT mutations, with the majority of these mutations at the D816 position of the KIT protein.

Disclosed herein is an improved process for the preparation of crystalline phosphate salt Form A of Compound (I):

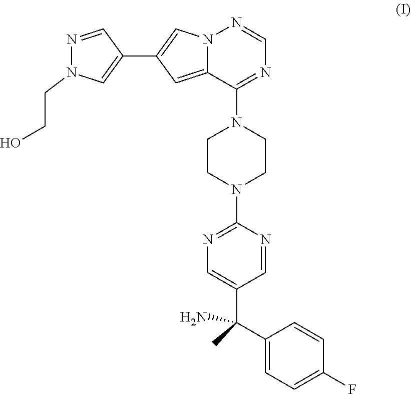 [SMILES: C[C@](N)(c1ccc(F)cc1)c1cnc(N2CCN(c3ncnn4cc(-c5cnn(CCO)c5)cc34)CC2)nc1]

comprising forming a phosphate salt of Compound (I) with phosphoric acid in an organic solvent mixture comprising 2-MeTHF/acetone/water. In one embodiment, the ratio of the organic solvent mixture is 1.0 volume 2-MeTHF: 1.0 volume acetone: 1.3-2.0 volume water. In one embodiment, the amount of phosphoric acid is 1.1 equivalents to 1.0 equivalents of Compound (I). In one embodiment, the process further comprises crystallizing phosphate salt Form A Compound (I) by adding acetone. In one embodiment, the acetone is added in over a period of 4-8 h.

The following examples are intended to be illustrative and are not intended to be limiting in any way to the scope of the disclosure.

### EXPERIMENTAL

### Abbreviations

| Abbre- | | Abbre- | |
|---|---|---|---|
| viation | Solvent | viation | Solvent |
| ACN | Acetonitrile | BA | Benzyl Alcohol |
| DCM | Dichloromethane | DEE | Diethyl ether |
| DMAc | N,N-Dimethylacetamide | | |
| DMF | N,N-Dimethylformamide | DMSO | Dimethylsulfoxide |
| EtOAc | Ethyl Acetate | EtOH | Ethanol |
| IPA | 2-Propanol | IPOAc/IPAc | Isopropyl acetate |
| MBK | Methyl Butyl Ketone | MCH | Methylcyclohexane |
| MEK | Methyl Ethyl Ketone | MeOAc | Methyl Acetate |
| MeOH | Methanol | MIBK | 4-Methyl-2-pentanone |
| MtBE | tert-Butyl Methyl Ether | NMP | N-Methyl Pyrrolidone |
| 1-PA | 1-Propanol | TFA | Trifluoroacetic Acid |
| IPA | 2-propanol | | |
| TFE | Trifluoroethanol | THF | Tetrahydrofuran |

Instruments

| Full Name | Abbreviation |
|---|---|
| Differential scanning calorimetry | DSC |
| High Performance Liquid Chromatography | HPLC |
| Karl Fischer Titration | KF |
| Nuclear Magnetic Resonance | NMR |
| X-ray Powder Diffraction | XRPD |
| Thermogravimetric Analysis | TGA |

Units

| Full Name | Abbreviation |
|---|---|
| Celsius | C. |
| Degrees | ° |
| Equivalents | eq. |
| Gram | g |
| Hour | H |
| Hertz (S⁻¹) | Hz |
| Milligrams | mg |
| Milliliters | mL |
| Minute | min |
| Relative Humidity | RH |
| Room temperature | RT |
| Second | sec |
| volume | vol. |
| Volume ratio | v/v |
| Weight | wt. |
| Weight Percentage | wt. % |

### Analysis Conditions

### X-Ray Powder Diffraction (XRPD)

### Bruker

XRPD of the freebase and phosphate salts disclosed herein was performed using a Bruker D8 Advance equipped with LYNXEYE detector in reflection mode (i.e. Bragg-Brentano geometry) except for Example 10 (conditions noted therein). Samples were prepared on Si zero-return wafers. The parameters for XRPD methods used are listed below:

| Parameter | Regular Scan | High Resolution Scan |
|---|---|---|
| X-ray wavelength | Cu Kα1, 1.540598 Å, | Cu Kα1, 1.540598 Å, |
| X-ray tube setting | 40 kV, 40 mA | 40 kV, 40 mA |
| Slit condition | 0.6 mm div. + 2.5° soller | 0.6 mm div. + 2.5° soller |
| Scan mode | Step | Step |
| Scan range (°2θ) | 4-30 | 4-40 |
| Step size (°2θ) | 0.03 | 0.02 |
| Dwell time (s/step) | 0.23 | 0.9 |
| Spin | Yes (0.5 Hz) | Yes (0.5 Hz) |

### Rigaku

X-ray powder diffraction of besylate, benzoate, and sulfate salts disclosed herein was performed using a Rigaku MiniFlex 600 in reflection mode (i.e. Bragg-Brentano geometry). Samples were prepared on Si zero-return wafers. The parameters for XRPD methods used are listed below:

| Parameter | Regular Scan | High Resolution Scan |
|---|---|---|
| X-ray wavelength | Cu Kα1, 1.540598 Å, | Cu Kα1, 1.540598 Å, |
| X-ray tube setting | 40 kV, 15 mA | 40 kV, 15 mA |
| Slit condition | 1.25° div., Ni kβ filter, | 1.25° div., Ni kβ filter, |
| | 0.3 mm rec. | 0.3 mm rec. |
| Scan mode | Continuous | Continuous |
| Scan range (°2θ) | 4-30 | 4-40 |
| Step size (°2θ) | 0.05 | 0.05 |
| Scan speed (°/min) | 5 | 1.25 |
| Spin | No | No |

### Simultaneous Thermogravimetric Analysis and Differential Scanning Calorimetry (TGA and DSC)

TGA and DSC were performed on the same sample simultaneously using a Mettler Toledo TGA/DSC³⁺. Protective and purge gas was nitrogen at a flowrate of 20-30 mL/min and 50-100 mL/min respectively. The desired amount of sample (5-10 mg) was weighed directly in a hermetic aluminum pan with pinhole and analyzed according to the parameters below:

Parameters

| Method | Ramp | |
|---|---|---|
| Sample size | 5-10 | mg |
| Heating rate | 10.0° | C./min |
| Temperature range | 30 to 300° | C. |

### Differential Scanning Calorimetry (DSC)

DSC was performed using a Mettler Toledo DSC³⁺. The sample (1-5 mg) was weighed directly in a 40 μL hermetic aluminum pan with a pinhole and analyzed according to the parameters below:

Parameters

| Method | Ramp | |
|---|---|---|
| Sample size | 3-5 | mg |
| Heating rate | 10.0° | C./min |
| Temperature range | 30 to 300° | C. |

| | |
|---|---|
| Method gas | N₂ at 60.00 mL/min |

Parameters

| Method | Modulation | |
|---|---|---|
| Sample size | 5-10 | mg |
| Amplitude | 1° | C. |
| Period | 60 | s |
| Heating rate | 2.0° | C./min |
| Temperature range | 30 to 300° | C. |

| | |
|---|---|
| Method gas | N₂ at 60.00 mL/min |

### ¹H-Nuclear Magnetic Resonance Spectroscopy (¹H-NMR)

Proton NMR was performed on a Bruker Avance 300 MHz spectrometer. Solids were dissolved in 0.75 mL deuterated solvent in a 4 mL vial, transferred to an NMR tube (Wilmad 5 mm thin wall 8″ 200 MHz, 506-PP-8) and analyzed according to the following parameters:

Parameters - Bruker Avance 300

| Instrument | Bruker Avance 300 MHz spectrometer | |
|---|---|---|
| Temperature | 300 | K |
| Probe | 5 mm PABBO BB-1H/DZ-GRD Z104275/0170 | |
| Number of scans | 16 | |
| Relaxation delay | 1.000 | s |
| Pulse width | 14.2500 | μs |
| Acquisition time | 2.9999 | s |
| Spectrometer frequency | 300.15 | Hz |

| | |
|---|---|
| Nucleus | ¹H |

### High Performance Liquid Chromatography (HPLC)

HPLC was conducted using an Agilent 1220 Infinity LC. Flow rate range of the instrument is 0.2-5.0 mL/min, operating pressure range is 0-600 bar, temperature range is 5° C. above ambient to 60° C., and wavelength range is 190-600 nm.

HPLC was conducted using an Agilent 1220 Infinity 2 LC equipped with diode array detector (DAD). Flow rate range of the instrument is 0.2-5.0 mL/min, operating pressure range is 0-600 bar, temperature range is 5° C. above ambient to 60° C., and wavelength range is 190-600 nm.

HPLC was conducted using a Hitachi LaChrom HPLC. Flow rate range of the instrument is 0.2-10.0 mL/min, operating pressure range is 0-412 bar, temperature range is 5° C. above ambient to 60° C., and wavelength range is 190-600 nm.

The HPLC method used in this study is shown below:

Parameters

| Mobile Phase A | 20 mM ammonium acetate solution in distilled | | |
|---|---|---|---|
| | water (pH 8.2) | | |
| Mobile Phase B | HPLC grade methanol | | |
| Diluent | ACN:water (85:15 vol) | | |
| Injection Volume | 5 μL | | |
| Monitoring Wavelength | 251 nm | | |
| Column | XBridge C-18, 4.6 × 150 mm, 3.5 μm | | |
| Column Temperature | 55° C. | | |
| Gradient Method | Time (min) | % A | Flow rate (mL/min) |
| | 0 | 75 | 0.8 |
| | 6 | 48 | 0.8 |
| | 20 | 48 | 0.8 |
| | 33 | 10 | 0.8 |
| | 39 | 10 | 0.8 |
| | 39.1 | 75 | 0.8 |
| | 45.0 | 75 | 0.8 |

### Karl Fischer Titration

KF titration for water determination was performed using a Mettler Toledo C20S Coulometric KF Titrator equipped with a current generator cell with a diaphragm, and a double-platinum-pin electrode. The range of detection of the instrument is 1 ppm to 5% water. Aquastar™ CombiCoulomat fritless reagent was used in both the anode and cathode compartments. Samples of approximately 0.03-0.10 g were dissolved in the anode compartment and titrated until the solution potential dropped below 100 mV. Hydranal 1 wt. % water standard was used for validation prior to sample analysis.

### Microscopy

Optical microscopy was performed using a Zeiss AxioScope A1 digital imaging microscope equipped with 2.5×, 10×, 20×, and 40× objectives and polarizer. Images were captured through a built-in Axiocam 105 digital camera and processed using ZEN 2 (blue edition) software provided by Zeiss. Optical microscopy was performed using a Hund Wetzlar Wilovert 30 inverted microscope equipped with a MiniVID USB 2.0, 5.1MP digital camera.

### Example 1: Preparation of Salts of Compound (I)

A stock solution with concentration of 85 mg/mL was prepared in TFE. Stock solutions of fourteen different counter ions (e.g., benzenesulfonic acid, benzoic acid, phosphoric acid, sulfuric acid, citric acid, glucuronic acid, glutamic acid, hydrochloric acid, malic acid, methanesulfonic acid, Succinic acid, tartaric acid, toluenesulfonic acid, and acetic acid) were prepared in EtOH. 352 μL (30 mg) of the freebase solution was added to each vial. 1.1 and/or 2.2 equivalents of the appropriate counter ion stock solution were added to each vial. The vials were left to stir at 45° C. for two hours before being uncapped and left to evaporate at 40° C. with stirring overnight in atmosphere. The vials were then placed under active vacuum at 50° C. for 3 hours to dry thoroughly. Approximately, 20 volumes (600 μL) of screening solvent were then added to each vial, and the samples were heated to 45° C. with stirring (450 rpm). Vials demonstrating significant precipitate, or gumming, were vortexed often to ensure thorough mixing. After two hours, the samples were left to stir at room temperature. When slurries were observed at room temperature, solids were filtered for characterization.

Similar experiments were also conducted in EtOAc and IPA:water (9:1 vol.).

From this salt screen, crystalline salts were observed for benzenesulfonic acid, benzoic acid, hydrochloric acid, methanesulfonic acid, sulfuric acid, succinic acid, and phosphoric acid.

Crystalline salts were characterized and evaluated for viability based on melting point, crystallinity, stability on drying and humidity exposure, water solubility, polymorphism, and acceptability of counter-ion.

Besylate, benzoate, and phosphate were all selected to be scaled up for further investigation as they were stable to drying and/or re-drying post humidification, and all demonstrated melting above 150° C. The sulfate was also chosen for further investigation.

In contrast, the solids isolated from screening with maleic acid and acetic acid were not selected as they were not confirmed to be salts. Although all gave stable crystalline patterns throughout screening, counter ion stoichiometry was not observed by NMR for both the acetate and malate. The malate also had a visibly different appearance after humidification and demonstrated low melting, as well as poor water solubility.

HCl was not selected due to poor physicochemical properties despite being a desirable counter-ion; both the observed crystalline pattern and amorphous solids deliquesced at high humidity. Mesylate and tosylate were not selected due to poor acceptability of counter-ion, their low crystallinity, as well as the solids (amorphous or crystalline) deliquescing upon humidification. Sulfuric acid demonstrated 2 patterns which were both stable to humidification, however, 9-A demonstrated poor thermal behavior, and 9-B became a hard tack-like solid post humidification.

### Example 2: Preparation of Amorphous Phosphate Salt of Compound (I)

Amorphous material was generated by lyophilization from water and t-BuOH:water (6:4 vol.). About 540 mg of phosphate salt of Compound (I) Form A (Example 5) was weighed into 20 mL scintillation vials followed by the addition of the solvent. For water, 20 mL was added in three steps while stirring at RT, but dissolution was not obtained. The vial was transferred onto a hot plate at 75° C. and complete dissolution was achieved within few minutes. However, the solution precipitated back after about 10 min of stirring. For t-BuOH:water, the initial mixture ratio was (9:1 vol.) but it was readjusted to (6:4 vol.) in order to achieve dissolution at 75° C. The solution was transferred to a 300 mL beaker and in total about 500 vol. of t-BuOH:water (6:4 vol.) were added.

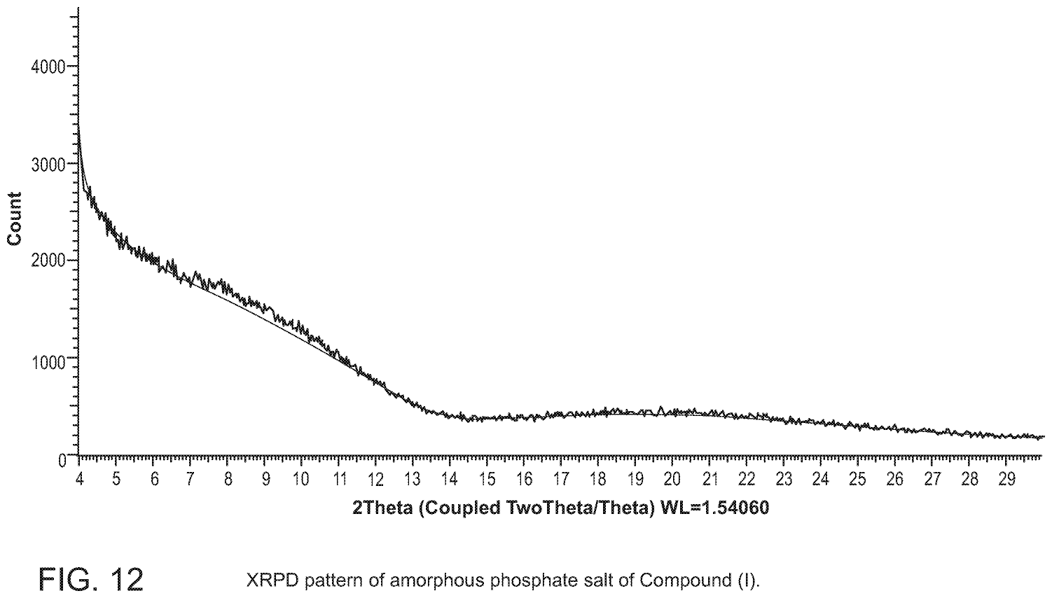

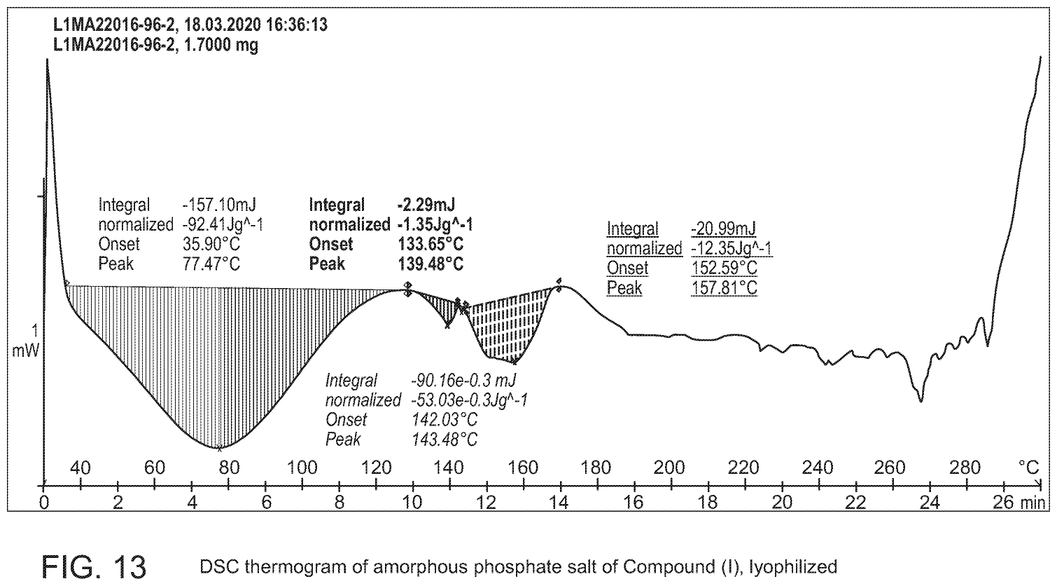

The solution was divided into 20 mL vials and each was dipped for approximately 2 min in liquid nitrogen to freeze. The solid was transferred to a freeze dryer and kept overnight. XRPD analysis of the solids generated by lyophilization confirmed amorphous pattern. See FIG. 12.

### Example 3: Preparation and Characterization of Phosphate Salt of Compound (I) Form G

### Amorphous Slurry Experiment:

About 62 mg of amorphous material (phosphate salt of Compound (I)) was weighed into a 4 mL vial and 1 mL of IPA:water (9:1 vol.) was added while stirring at RT. A clear solution with a gum at the bottom of the vial was formed and the gum was not broken by sonication or vortexing. The sample was left to stir at RT for 2 days after which it changed to a thick, flowable white slurry. An aliquot was taken and XRPD analysis confirmed Form G+Form A. After 9 days on the stir plate at RT. XRPD analysis confirmed a highly crystalline Form G. Form G became less crystalline after drying under static vacuum at 50° C. overnight and a sample of high-crystalline G left on the XRPD plate at ambient conditions became less crystalline after 4 days.

### Characterization

Simultaneous TGA/DSC thermograms of crystalline Pattern G (wet) showed a mass loss of 30 wt. % between 40 and 140° C. with three endothermic events within this temperature range, FIG. 2C. A fourth endothermic peak that corresponded to melting was observed with onset temperature at 149.6° C. and was in agreement with the endothermic peak of Pattern G obtained in the previous work with onset temperature at 144.7° C., FIG. 2D.

DSC and NMR analysis show that Form G is a hydrate (mass loss at <100° C. observed by DSC, and only 0.3 wt. % IPA by NMR). KF analysis determined 3.75 wt. % water in the sample confirming 1.3 molar equivalents of water within the sample.

Form G was subjected to 40° C. and 75% relative humidity for 7 days to test its stability. About 10 mg of phosphate salt of Compound (I) Form G was weighed into a 4 mL vial and covered with a KimWipe. The vial was placed into a 20 mL vial containing saturated aqueous NaCl. The system was placed on a hot plate at 40° C., creating an atmosphere of 75% relative humidity, for 7 days. The solid was then plated for XRPD analysis. After one week, some peak shifts in XRPD were observed, these shifts correspond well with Form O, although they are not as well resolved.

### Example 4: Preparation and Characterization of Phosphate Salt of Compound (I) Form O

Experiments using a polymer to promote precipitation were completed using the two solvent systems and two different polymers, Table 45. About 30 mg of phosphate salt of Compound (I) Form A was weighed into 2 mL vials and the solvent was added while stirring at RT. For acetone:water (7:3 vol.), 1.4 mL was added to achieve complete dissolution and for THF:water (7:3 vol.), 400 μL was added. Polyethylene glycol (PEG) and polyvinylpyrrolidone (PVP) polymers were added incrementally, one spatula tip at a time, while monitoring for precipitation. The solutions became more viscous with polymer addition and changed to thick, clear viscous solutions and no precipitation was observed in all four samples. The samples were left on the stir plate and after eight days a thick white slurry formed in the sample prepared in THF:water (7:3 vol.) with PEG. XRPD analysis on the wet cake collected from this sample confirmed Form O.

TABLE 45
Summary of polymer crystallization experiments.

| | Poly- | | |
|---|---|---|---|
| Solvent | mer | XRPD | Comments |
| Acetone:water | PEG | O (wet) | No precipitation after adding more than |
| (7:3 vol.) | | | 40 spatula tips of polymer. Clear, |
| | | | viscous solution. Changed to thick |
| | | | slurry after 8 days. |
| THF:water | PEG | | No precipitation after adding more than |
| (7:3 vol.) | | | 20 spatula tips of polymer. Clear, |
| | | | viscous solution. |
| Acetone:water | PVP | | No precipitation after adding more than |
| (7:3 vol.) | | | 40 spatula tips of polymer. Clear, |
| | | | viscous solution. |
| THF:water | PVP | | No precipitation after adding more than |
| (7:3 vol.) | | | 20 spatula tips of polymer. Clear, |
| | | | viscous solution. |

Blank cells indicate no data were available.

### Example 5: Preparation and Characterization of Phosphate Salt of Compound (I) Form A

### 5.1 Small Scale Preparation

399.4 mg of Compound (I) (free base) was weighed into a 20 mL scintillation vial and a stir bar was added. 10 volumes (4 mL) of TFE was added to dissolve the solid at 45° C. with stirring. 1.1 equivalents (3.5 mL) of phosphoric acid stock solution was added dropwise to the solution of freebase, white precipitate was visible immediately. The solution continued to stir for 1 hour at 45° C. prior to removing the cap, adjusting the temperature to 40° C., and allowing the solvent to evaporate over a weekend. While stirring at 45° C., the white precipitate disappeared and only a peach colored gum was present. The gum would break apart with sonication and vortexing, however, the “ball of gum” would return quickly while stirring. The vial was then placed under active vacuum at 50° C. for 3 hours. Once removed, the vial contained off-white solids. 20 volumes of EtOH (8.0 mL) was added to the vial, and an off-white slurry appeared almost instantaneously. The slurry was allowed to stir at 45° C. or 1 hour, then RT overnight.

The off-white slurry was sampled for XRPD analysis and Form A was confirmed. The slurry was filtered, washed with 2×1.5 volumes (599 μL) of EtOH, and placed under vacuum at 50° C. to dry.

385.2 mg (81% yield counter ion corrected) of white solid was collected, an loss on drying (LOD) of 63% was observed.

TGA analysis demonstrated a gradual mass loss of 0.3 wt. % up to 120° C., followed by a loss of 1.9 wt. % up to 210° C. in correspondence with melting (FIG. 1B). The DSC demonstrated two endothermic events; a small endotherm with an onset of 161.3° C., and a larger endotherm with an onset of 191.8° C. (FIG. 1C).

The API:CI (counter-ion) ratio was not confirmed by ¹H NMR; however, the residual solvent was found to be 0.45 wt. % EtOH. Microscopy revealed small needles.

Purity for the phosphate was found to be 98.20% a/a by HPLC.

### 5.2 Large Scale Preparation

### 5.2.1

To a solution of Compound (I) (free base) (21.8 kg) in THF (405 kg) at 45-55° C. was filtered through a carbon filter. The resulting solution was heated to 60-70° C. and distilled removing 321 kg THF. To this concentrated solution 16.5 wt % phosphoric acid solution in water (27 kg, 1.1 eq) was added with the mixture at 40-50° C. A rinse of water (15 kg) was charged followed by further vacuum distillation at 40-50° C. removing approximately 40 kg. To the resulting mixture was charged acetone (24 kg) at 40-50° C. and then Compound (I) phosphate seeds (26 g). An additional charge of acetone (260 kg) was added and then the mixture cooled to 15-25° C. The Compound (I) phosphate salt was isolated by filtration washing twice with a mixture of acetone/water/THF (47 kg/6.4 kg/7.3 kg) resulting in 26.1 kg, >99% yield and 99.9% purity.

### 5.2.2

To a reactor R1 at ambient temperature was charged, with stirring, 2-MeTHF (52 mL), acetone (52 mL), water (70 mL), followed by addition of Compound (I) free base (25 g, 1 equiv.) and concentrated H₃PO₄ (6.7 g, 76.3 wt % assay, 1.1 equiv.). The mixture in R1 was heated to 50° C., then polish filtered into a separate reactor R2, which was preheated at ˜47° C. Acetone (8.7 mL) was added to R2 at ˜47° C., followed by addition of Compound (I) phosphate seed (0.25 g, 1 wt %). The mixture was stirred at ˜47° C. for 0.5 h. The remainder of acetone (450 mL) was added, with slow stirring, at the same temperature in 6 h (240 mL was added in the initial 4 h, then remaining 210 mL was added in 2 h). The resulting slurry was cooled to 20-25° C. for 18 h and filtered. The wet cake was washed with a mixture of 2-MeTHF:acetone:water (9:80:11, 38 mL×2), then dried under vacuum at 50° C. to give 27.5 g of Compound (I) phosphate as a solid, with 93% yield and 99.94% HPLC purity. XRPD analysis shows that the obtained phosphate salt of Compound (I) is crystalline Form A.

The original process to make Compound (I) phosphate salt Form A was performed in the solvent mixture THF/acetone/water. As shown in Table A, however, the residual THF in Compound (I) phosphate salt Form A prepared using this solvent mixture was close to, or above, the maximum accepted limits according to International Council for Harmonization (ICH) residual solvent guidelines for THF (≤720 ppm). THF is a Class II solvent considered to be limited in pharmaceutical products because of inherent toxicity per ICH guidelines.

TABLE A
Residual THF in Compound (I) hosphate Salt
Form A cGMP Batches Prepared with THF

| Batch # | 1 | 2 | 3 | 4 | 5 |
|---|---|---|---|---|---|
| Residual THF | 695 | 793 | 585 | 509 | 484 |
| (Specification: ≤720 | ppm | ppm (out of | ppm | ppm | ppm |
| ppm) | | specification) | | | |

Therefore, an improved process of making Compound (I) phosphate Form A was developed using 2-MeTHF, instead of THF, in the crystallization process. 2-MeTHF is a class III solvent (solvent with a low toxic potential) per ICH guidelines, with maximum accepted limit being 5000 ppm. The R&D data demonstrates that the newly developed 2-MeTHF process produces good-quality Compound (I) phosphate salt Form A, with robust control of residual 2-MeTHF (Table B).

TABLE B
Residual 2-MeTHF in Compound (I) Phosphate Salt
Form A cGMP Batches Prepared with 2-MeTHF

| Batch # | 1 | 2 | 3 | 4 |
|---|---|---|---|---|
| Residual 2-MeTHF | <7 ppm | 34 ppm | <7 ppm | 151 |
| (Specification: ≤5000 | | | | |
| ppm) | | | | |

### 5.2 Ratio of API to Counter-Ion for Phosphate Salt of Compound (I) Form A

To determine the ratio API to counter-ion, known concentrations of both the freebase of Compound (I) and phosphate of Compound (I) Form A were made up in volumetric flasks to concentrations of approximately 0.5 mg/mL. These samples were then injected for HPLC analysis. The total area counts under the main API peak were compared for strength assessment. The strength was determined to be 88%, which equates to 0.8 molar equivalents of phosphoric acid. Inductively coupled plasma atomic emission spectroscopy (ICP OES) was also completed on a sample of Form A. The phosphorous content was determined to be 4.65 wt. %, which is in good agreement to the theoretical phosphorous content of a mono-phosphate, 4.94 wt. %.

### 5.3 Stability Test

### A. Short-Term Slurries

Short-term slurries were carried out following the gravimetric solubility assessment at two temperatures in 14 solvents. Vials containing slurries were centrifuged and settled solids were recovered and filtered for XRPD analysis. A summary of results is presented in Table 46. Samples prepared in NMP, DMAc, and DMSO at RT remained in solutions and no solids were collected. All other 23 samples formed slurries and Form A was obtained from 21 samples. Freebase Form A (FB-A)+Phosphate Salt Form A was obtained in water at 50° C., indicating a disproportionation of the phosphate salt. The pattern obtained in TFE at 50° C. was from a degradation by-product, as confirmed by HPLC analysis.

TABLE 46
Summary of XRPD patterns obtained from
the short-term slurry experiments.

XRPD (wet cake)

| Solvent | RT | 50° C. |
|---|---|---|
| MeOH | A | A |
| MeOAc | A | A |
| MEK | A | A |
| Water | A | FB-A + A |
| NMP | No solid collected | A |
| TFE | A | Pattern from a |
| | | degradation by- |
| | | product |
| DMF | A | A |
| DMAc | No solid collected | A |
| DMSO | No solid collected | — |
| Anisole | A | A |
| Chloroform | A | — |
| Chlorobenzene | A | A |
| Benzyl alcohol | A | A |
| EtOH:TFE (8:2 vol.) | A | A |

### B. Long-Term Slurries and Slow Evaporation

Slow evaporation and long-term slurries experiments were set up in 10 different solvents/solvent systems, Table 47. About 8 mg of phosphate salt of Compound (I) Form A was weighed into 2 mL HPLC vials and 1.5 mL of solvent was added while stirring at 35° C.

Solids in acetone:water (7:3 vol.) and ACN:water (8:2 vol.) were completely dissolved. These samples were set up for crystallization by slow evaporation. A small gauge needle was inserted in the vials caps and the solvent was left to evaporate while stirring at 35° C. Samples in neat solvents were thin slurries and solids did not completely dissolve. These samples were used for long-term slurries. Water (75 μL) was added to some samples (Table 47) and samples in ACN, THF, and MeOAc were transferred to a cold plate at 10° C. in order to probe for possible hydrate formation. After 10 days, the samples in neat solvents were filtered and analyzed by XRPD. Phosphate Form A was obtained for all samples. Free base Form FB-A was obtained from the two slow-evaporation samples.

TABLE 47
Summary of results from long-term slurries and slow evaporation.

| | T | | |
|---|---|---|---|
| | (° | XRPD | |
| Solvent | C.) | pattern | Comments/Observations |
| Dioxane | 35 | A | Slurry. Analyzed by XRPD after 11 days. |
| ACN:water | 10 | A | Slurry. About 75 μL water was added |
| (20:1 vol.) | | | after solvent addition. Analyzed by |
| | | | XRPD after 11 days. |
| Toluene:water | 35 | A | Slurry. About 75 μL water was added |
| (20:1 vol.) | | | after solvent addition. Analyzed by |
| | | | XRPD after 11 days. |
| THF:water | 10 | A | Slurry. About 75 μL water was added |
| (20:1 vol.) | | | after solvent addition. Analyzed by |
| | | | XRPD after 11 days. |
| EtOAc:water | 35 | A | Slurry. About 75 μL water was added |
| (20:1 vol.) | | | after solvent addition. Analyzed by |
| | | | XRPD after 11 days. |
| MtBE | 35 | A | Slurry. Analyzed by XRPD after 11 days. |
| 2-MeTHF | 35 | A | Slurry. Analyzed by XRPD after 11 days. |
| MeOAc:water | 10 | A | Slurry. About 75 μL water was added |
| (20:1 vol.) | | | after solvent addition. Analyzed by |
| | | | XRPD after 11 days. |
| Acetone:water | 35 | FB-A | Complete dissolution. Slow evaporation |
| (7:3 vol.) | | (small | to dryness. |
| | | sample | |
| | | amount) | |

### C. Milling

Dry and solvent drop milling was done using a Wig-L-Bug ball mill with ¼″ stainless steel ball as milling media. About 30 mg solid (phosphate salt of Compound (I) Form A) was weighed into each vessel and one volume solvent was added. The milling was carried out in 3×30 s increments at 3,800 rpm, scraping solids off vessel walls to minimize caking between millings. The summary of patterns obtained is shown in Table 48. XRPD analysis showed that the solid obtained from dry milling had amorphous+trace Form A.

TABLE 48
Results from dry and solvent drop milling experiments.

| | XRPD | XRPD | |
|---|---|---|---|
| | pattern | pattern | Observations on |
| Solvent | (initial) | (final) | XRPD plate |
| None (dry) | A | Am. + trace A | Dry solid |
| EtOH | | A | Thick paste, felt dry |
| DMAc | | A | Wet paste |
| MEK | | A | Dry solid |
| MeOAc | | A | Slightly wet solid |
| Dioxane | | A | Paste |
| Acetone:water | | A | Dry solid |
| (7:3 vol.) | | | |

Am., amorphous

### Example 6: Preparation and Characterization of Besylate Salt of Compound (I)

### 6.1 Preparation of Besylate Form 1-A

386.5 mg of Compound (I) freebase was weighed into a 20 mL scintillation vial and a stir bar was added. 10 volumes (4 mL) of TFE was added to dissolve the solid at 45° C. with stirring. 1.1 equivalents (4.3 mL) of benzenesulfonic acid stock solution was added dropwise to the solution of freebase, no visible changes occurred. The solution continued to stir for 1 hour at 45° C. prior to removing the cap, adjusting the temperature to 40° C., and allowing the solvent to evaporate over a weekend. The vial was then placed under active vacuum at 50° C. for 3 hours. Once removed, the vial contained a thin orange gel/film on the vial walls with no visible solids present. 20 volumes of EtOH (7.7 mL) was added to the vial, and an off-white slurry appeared almost instantaneously. Some orange crust was visible on the walls. The slurry was allowed to stir at 45° C. for 1 hour, then RT overnight.

The off-white slurry was sampled for XRPD analysis, besylate Form 1-A was confirmed. The slurry was filtered, washed with 2×1.5 volumes (580 μL) of EtOH, and placed under vacuum at 50° C. to dry. 98.4 mg (20% yield, counter ion corrected) of white solids were collected, a LOD (loss on drying) of 71% was observed.

TGA demonstrated a mass loss of 0.9 wt. % up to 120° C., and 0.8 wt. % up to 200° C. (FIG. 4B). Only one endothermic event was observed in the DSC thermogram, having an onset of 189.4° C. (FIG. 4C). Stoichiometry was confirmed to be approximately 1:2 API:CI by ¹H NMR. Due to several overlapping peaks, the exact stoichiometry is difficult to obtain. The residual solvent was found to be 0.17 wt. % EtOH.

Purity for the besylate scale up was found to be 93.12% a/a by HPLC.

### 6.2 Preparation of Besylate Form 1-B

Besylate Form 1-B was formed after besylate Forma 1-A was subjected to a humidity test and re-dried.

### Example 7: Preparation and Characterization of Benzoate Salt of Compound (I)

### 7.1 Preparation of Benzoate Form 2-A

389.8 mg of Compound (I) freebase was weighed into a 20 mL scintillation vial and a stir bar was added. 10 volumes (4 mL) of TFE was added to dissolve the solid at 45° C. with stirring. 1.1 equivalents (3.5 mL) of benzoic acid stock solution was added dropwise to the solution of freebase, no visible changes occurred. The solution continued to stir for 1 hour at 45° C. prior to removing the cap, adjusting the temperature to 40° C., and allowing the solvent to evaporate over a weekend. The vial was then placed under active vacuum at 50° C. for 3 hours. Once removed, the vial contained off-white solids as well as an orange crust on the vial walls. 20 volumes of EtOAc (7.8 mL) was added to the vial, and an off-white slurry appeared almost instantaneously. Some orange crust was visible on the walls. The slurry was allowed to stir at 45° C. or 1 hour, then RT overnight.

The off-white slurry was sampled for XRPD analysis, benzoate Form 2-A was confirmed. The slurry was filtered, washed with 2×1.5 volumes (585 μL) of EtOAc, and placed under vacuum at 50° C. to dry. 347.1 mg (72% yield, counter ion corrected) of white solids were collected, an LOD of 54.7% was observed.

TGA analysis demonstrated a mass loss of 0.2 wt. % up to 120° C., followed by a loss of 5.4 wt. % up to 200° C. (with melting), FIG. 6B. The DSC thermogram demonstrated two endotherms at 171.3° C. and 180.8° C., FIG. 6C. Stoichiometry was confirmed to be 1:1 API:CI by ¹H NMR, and the residual solvent was found to be 0.83 wt. % EtOAc. Microscopy revealed small rods and needles.

Purity for the benzoate was found to be 96.53% a/a by HPLC.

### 7.2 Preparation of Benzoate Form 2-B

Benzoate Form 2-B was formed after benzoate Forma 2-A was subjected to a humidity test and re-dried.

### Example 8: Preparation and Characterization of Sulfate Salt of Compound (I)

A sulfuric acid stock solution with concentration of 85 mg/mL in IPA:water (9:1 vol.) was prepared in TFE. 352 μL (30 mg) of the freebase solution was added to a vial. 1.1 equivalents of the sulfuric acid stock solution were added to the vial. The vials were left to stir at 45° C. for two hours before being uncapped and left to evaporate at 40° C. with stirring overnight in atmosphere. The vials were then placed under active vacuum at 50° C. for 3 hours to dry thoroughly. Approximately, 20 volumes (600 μL) of screening solvent were then added to the vial, and the sample was heated to 45° C. with stirring (450 rpm). The samples were left to stir at room temperature. Solids were filtered for characterization. XRPD analysis shows sulfate Form 9-A was formed.

### Example 9: Amorphous Free Base of Compound (I)

The solubility of Compound (I) free base was tested in a variety of solvents (Table 49).

TABLE 49

| | | | Solvent | | |
|---|---|---|---|---|---|
| | | | added | Solubility | |
| | Mass | | (# vol.) | (mg/mL) | XRPD |
| Sample | (mg) | Solvent | at 50° C. | at 50° C. | pattern |
| 82-1 | 16.5 | ACN:water | 96 | N/A | N/A |
| | | (1:1 vol.) | | | |
| 82-2 | 16.4 | ACN:water | 116 | 8.6 | Amorphous |
| | | (7:3 vol.) | | | |
| 82-3 | 16.7 | ACN:water | 96 | | N/A |
| | | (3:7 vol.) | | | |
| 83-1 | 30.4 | t-BuOH | 385 | <2.6 | Amorphous |
| | | | | | (+L.C) |
| 83-2 | 30.1 | t-BuOH:water | 385 | <2.6 | Amorphous |
| | | (9:1 vol.) | | | |

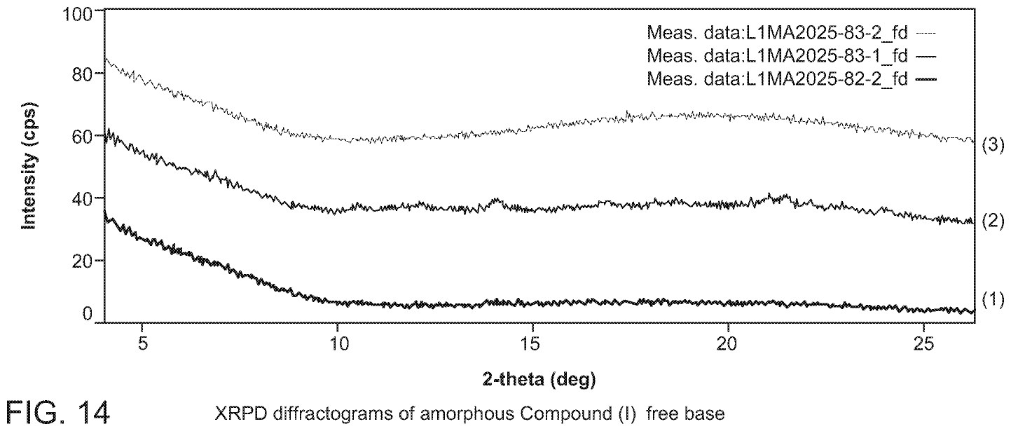

The solubility of Compound (I) free base was relatively better in acetonitrile (ACN):water (7:3 vol.), tert-butanol (t-BuOH), and t-BuOH:water (9:1 vol.). These solutions were filtered by syringe filtration (samples 83-1 and 83-2) before being used for the lyophilization experiment. The samples were first frozen in liquid nitrogen and then placed in the vacuum system of the freeze dryer (0.045 mbar, −89° C. collector temperature). The drying in vacuum at RT took place overnight and then samples were removed and prepared for XRPD analysis. The XRPD (see FIG. 14) were recorded after a day where the samples were stored in vials wrapped with a parafilm and left on the bench at RT.

### Example 10: Preparation of Compound (I) Free Base

### Synthesis of (S)-2-(4-(4-(4-(5-(1-amino-1-(4-fluorophenyl)ethyl)pyrimidin-2-yl)piperazin-1-yl)pyrrolo[1,2-f][1,2,4]triazin-6-yl)-1H-pyrazol-1-yl)ethanol

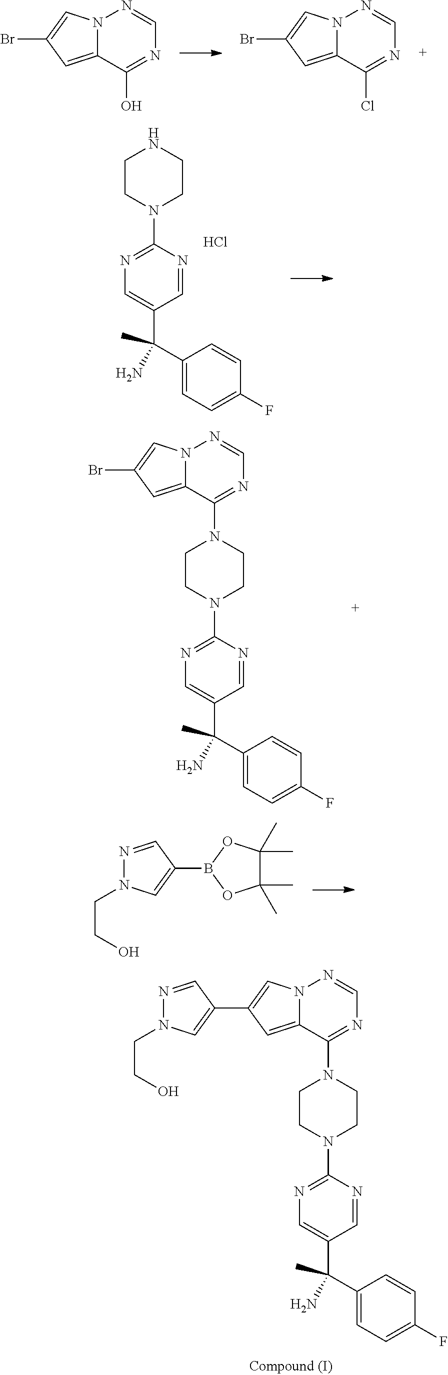

### Synthesis of 6-bromo-4-chloropyrrolo[2,1-f][1,2,4]triazine

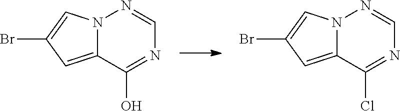 [SMILES: Oc1ncnn2cc(Br)cc12 ; Clc1ncnn2cc(Br)cc12]

A mixture of 6-bromopyrrolo[2,1-f][1,2,4]triazin-4-ol (10.0 g, 46.7 mmol), phosphorus oxychloride (13.0 mL, 140 mmol) and triethylamine (13.0 mL, 93.5 mmol) in toluene (100 mL) was stirred at 120° C. for 18 h. The reaction mixture was concentrated. The residue was purified by flash chromatography on silica gel eluting with EtOAc/petroleum ether 1:10 to 1:2 to give the title compound (9.4 g, 87% yield) as a pale yellow solid. MS (ES+) C₆H₃BrClN₃ requires: 231, found: 232, 234 [M+H]⁺.

### Synthesis of (S)-1-(2-(4-(6-bromopyrrolo[1,2-f][1,2,4]triazin-4-yl)piperazin-1-yl)pyrimidin-5-yl)-1-(4-fluorophenyl)ethanamine

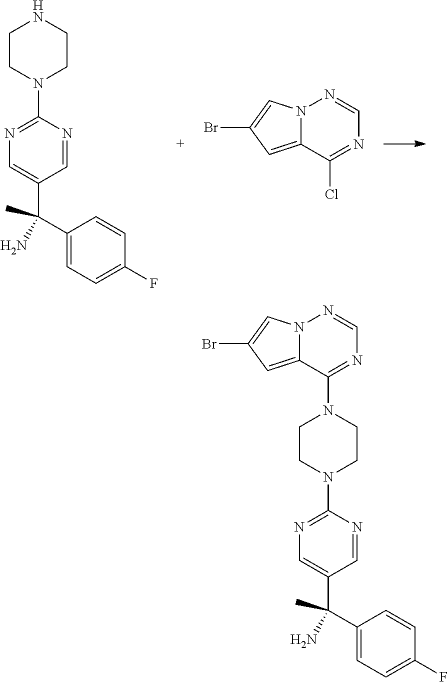 [SMILES: C[C@](N)(c1ccc(F)cc1)c1cnc(N2CCNCC2)nc1 ; Clc1ncnn2cc(Br)cc12 ; C[C@](N)(c1ccc(F)cc1)c1cnc(N2CCN(c3ncnn4cc(Br)cc34)CC2)nc1]

A mixture of (S)-1-(4-fluorophenyl)-1-(2-(piperazin-1-yl)pyrimidin-5-yl)ethanamine hydrochloride (2.00 g, 6.63 mmol), 6-bromo-4-chloropyrrolo[1,2-f][1,2,4]triazine (2.31 g, 9.94 mmol) and triethylamine (2.00 g, 19.8 mmol) in dioxane (20 mL) was stirred at RT for 2 h. LC-MS showed the reaction was full conversion. The mixture was purified by flash column chromatography (DCM/MeOH=20/1) to afford the title compound (1.51 g, 45% yield) as a white solid. MS (ES+) C₂₂H₂₂BrFN₈ requires: 496, found: 480 [M-16]⁺.

### Synthesis of (S)-2-(4-(4-(4-(5-(1-amino-1-(4-fluorophenyl)ethyl)pyrimidin-2-yl)piperazin-1-yl)pyrrolo[1,2-f][1,2,4]triazin-6-yl)-1H-pyrazol-1-yl)ethanol (Compound (I))

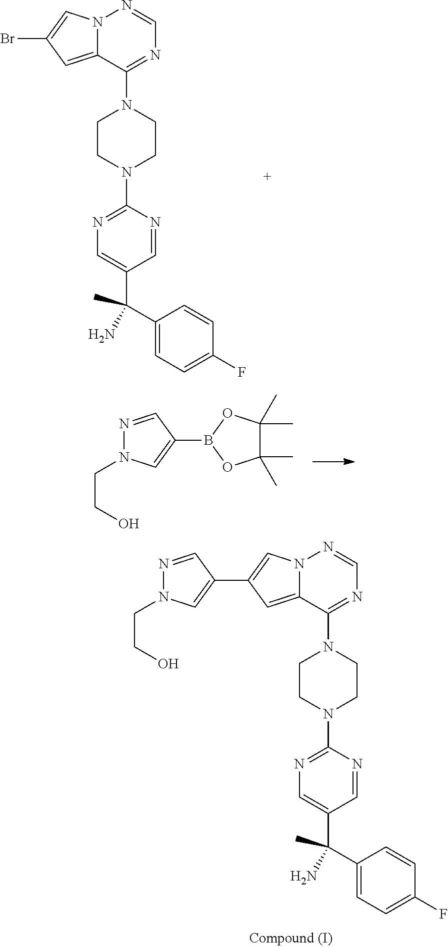

A mixture of (S)-1-(2-(4-(6-bromopyrrolo[1,2-f][1,2,4]triazin-4-yl)piperazin-1-yl)pyrimidin-5-yl)-1-(4-fluorophenyl)ethanamine (500 mg, 1.00 mmol), 2-(4-(4,4,5,5-tetramethyl-1,3,2-dioxaborolan-2-yl)-1H-pyrazol-1-yl)ethanol (285 mg, 1.20 mmol), Pd(dppf)Cl₂ (219 mg, 300 μmol) and Na₂CO₃ (317 mg, 3.00 mmol) in dioxane/water (20 mL/2 mL) was stirred at 100° C. for overnight under N₂. LC-MS showed the reaction was full conversion. The mixture was purified by flash column chromatography (DCM/MeOH=15/1) and Prep-HPLC (Mobile phase: A=water (0.1% NH₄HCO₃), B=acetonitrile; Gradient: B=15%-95% in 18 min; Column: Xtimate 10 um 150 A 21.2×250 mm) to afford the title compound (154.0 mg, 29% yield) as a white solid. MS (ES+) C₂₇H₂₉FN₁₀O requires: 528, found: 529 [M+H]⁺. ¹H-NMR (400 MHZ, 6d-DMSO) δ ppm 8.40 (s, 1H), 8.07 (s, 1H), 8.00 (s, 1H), 7.87 (s, 1H), 7.84 (s, 1H), 7.49-7.44 (m, 2H), 7.24 (s, 1H), 7.14-7.07 (m, 2H), 4.95 (t, 1H, J=5.2 Hz), 4.16-4.12 (m, 2H), 4.11-4.07 (m, 4H), 3.92-3.88 (m, 4H), 3.77-3.72 (m, 2H), 2.44 (br. S., 2H), 1.73 (s, 3H).

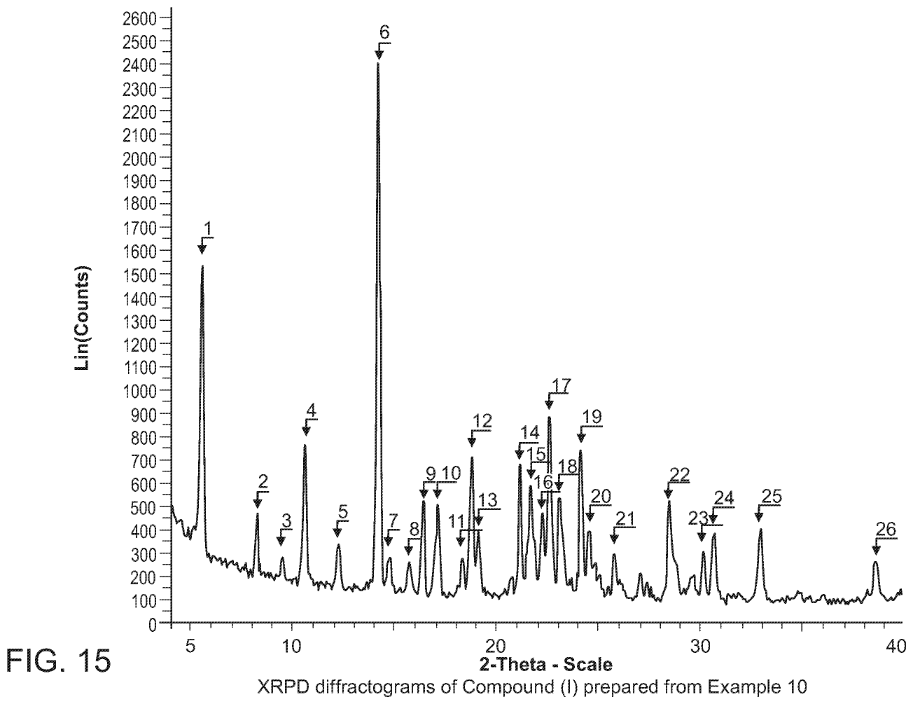

The XRPD spectrum (FIG. 15) and peaks (Table 50) of the obtained product are shown below.

TABLE 50

| 2-theta (deg) | d-Spacing (ang.) | Intensity (Count) |
|---|---|---|
| 5.48 | 16.11 | 1532 |
| 8.19 | 10.79 | 467 |
| 9.41 | 9.39 | 287 |
| 10.56 | 8.37 | 758 |
| 12.17 | 7.27 | 335 |
| 14.14 | 6.26 | 2403 |
| 14.69 | 6.03 | 284 |
| 15.69 | 5.64 | 262 |
| 16.35 | 5.42 | 519 |
| 17.11 | 5.18 | 503 |
| 18.30 | 4.84 | 276 |
| 18.78 | 4.72 | 712 |
| 19.08 | 4.65 | 403 |
| 21.15 | 4.20 | 672 |
| 21.69 | 4.09 | 584 |
| 22.26 | 3.99 | 471 |
| 22.58 | 3.93 | 881 |
| 23.07 | 3.85 | 533 |
| 24.17 | 3.68 | 739 |
| 24.55 | 3.62 | 391 |
| 25.80 | 3.45 | 289 |
| 28.55 | 3.12 | 517 |
| 30.22 | 2.95 | 303 |
| 30.71 | 2.91 | 380 |
| 33.02 | 2.71 | 399 |
| 38.73 | 2.32 | 262 |

### Example 11: Interconversion Among Freebase Crystalline Forms

The same crystalline form of free base Compound (I) may contain various amounts of water (i.e., channel hydrate) and is therefore characterized as different sub-forms, e.g., Forms FB-A-0 (monohydrate), FB-A-1 (metastable hydrate), and FB-A-2 (dehydrate). Crystalline Forms FB-A, FB-A-1, and FB-A-2 can be interconverted among each other. For example, Form FB-A-1 can be obtained from Form FB-A by slurrying in MeOH or EtOH at 50° C., or by slurrying and fast cooling in DCM:MeOH (40:60 vol.). Form FB-A-1 can be converted into Form FB-A under humidification at 75% RH at 40° C.

Form FB-A-2 can be obtained from Form FB-A-0 by drying at 50° C. under vacuum for three days. The nitrogen was purged when sample was removed from the oven and immediately the sample was covered with the airtight holder and XRPD analysis was conducted.

### Example 12: A Phase 1, Randomized, Double-Blind, Placebo-Controlled, Single and Multiple Ascending Dose Study to Evaluate the Safety, Tolerability, and Pharmacokinetics of Compound (I)

Parts 1 and 2 are randomized, double-blind, placebo-controlled investigation of SAD (Part 1) and MAD (Part 2) of orally administered Compound (I) in healthy adult subjects. Compound (I) was administered as the monophosphate salt. Screening of subjects occurred within 28 days prior to (first) dosing. Subjects participated in only 1 cohort in only 1 study part.

Part 1 (SAD): Six (6) cohorts of 8 subjects each (6 active and 2 placebo) were evaluated. Cohorts of subjects aged ≥18 years received either oral placebo (n=2/cohort) or single oral doses of Compound (I) (n=6/cohort) of 15, 25, 50, 100, or 200 mg under fasting conditions. The first cohort (Cohort S1) included a sentinel group (1 active and 1 placebo) which was dosed at least 24 hours before the remaining 6 subjects (5 active and 1 placebo). Dosing of the remaining 6 subjects in Cohort S1 was conducted following a safety evaluation of the sentinel group.

Plasma samples were collected pre-dose and through 96 hours following Compound (I) or placebo administration for pharmacokinetic (PK) assessment. Blood samples were collected pre-dose on Day-1, on Day 2, and on Day 5 to measure pharmacodynamic (PD) biomarker (tryptase) concentration. Cardiodynamic ECG sampling was collected pre-dose and through 24 hours following Compound (I) administration.

Dose escalation to the next cohort took place once the safety review committee (SRC) determined that adequate safety and tolerability had been demonstrated.

Part 2 (MAD): Four (4) cohorts of 8 subjects each (6 active and 2 placebo) were evaluated. In each cohort, subjects received a daily oral dose of Compound (I) at 25, 50, or 100 mg or placebo in a 3:1 ratio under fasting conditions for 10 consecutive days.

Plasma samples were collected pre-dose on Days 1-10 for PK assessment. Plasma samples were also collected through 24 hours post-dose on Day 1 and through 96 hours post-dose on Day 10 for PK assessment. Blood samples were collected pre-dose on Day 1, and pre-dose on Days 2, 5, and 9 to measure PD biomarker (tryptase) concentration. Cardiodynamic ECG sampling was collected pre-dose and through 24 hours following Compound (I) administration on Day 1 and Day 10. Safety was assessed according to incidence and severity of adverse events in recipients of Compound (I) as compared with placebo.

Compound (I) was well-tolerated across the five Compound (I) SAD cohorts and three MAD cohorts. Only Grade 1 adverse events, mostly not related to the drug, were reported, including abdominal pain, decreased appetite, fatigue, headache and nausea. No serious AEs were reported and there were no clinically relevant findings in laboratory or vital signs parameters reported. After administration of Compound (I) at single doses, the median Tmax ranged from 1.5 to 6 h post-dose. The mean half-life (t1/2) of Compound (I) ranged from 20 h to 28 h, indicating steady state is expected to be reached by Day 7, thereby supporting once-daily dosing. After 10 days of repeated oral administration of 25 to 100 mg Compound (I), the geometric mean accumulation ratio for AUC ranged from 1.6 to 1.8. The geometric mean Vz/F ranged from 753 to 973 L, indicating wide tissue distribution. Overall, a dose-proportional increase in systemic exposure to Compound (I) was observed across SAD and MAD cohorts. The pharmacokinetics of Compound (I) were linear across the dose ranges in SAD and MAD cohorts.

### Example 13: A Randomized, Double-Blind, Placebo-Controlled Phase 2/3 Study of Phosphate Salt of Compound (I) Form A (Referred to as “Compound (I)” in This Example 13) in Indolent Systemic Mastocytosis

This is a randomized, double-blind, placebo-controlled, Phase 2/3 study comparing the efficacy and safety of Compound (I)+Best Supportive Care (BSC) with placebo+BSC in patients with ISM whose symptoms are not adequately controlled by BSC. In Part 1, the recommended dose (RD) of Compound (I) is identified in patients with ISM who have an ISM-SAF TSS≥28. In Part 2, patients with ISM, regardless of ISM-SAF TSS, are randomly assigned to the RD of Compound (I) identified in Part 1+BSC or to matching placebo+BSC. In Part M, patients with mMCAS receive the RD of open-label Compound (I). In Part 3, patients who have completed Part 1 or Part 2 of the study participate in a long-term extension, receiving open-label at the RD+BSC.

### Screening (All Parts)

After provision of written informed consent, patients are evaluated for eligibility during the Screening period.

TSS eligibility for Part 1 is determined by the daily ISM-SAF averaged over a 14-day period. Patients meeting the threshold for symptom severity continue completing the ISM-SAF daily without interruption through screening and study participation if deemed otherwise eligible. After ISM-SAF symptom eligibility in Part 1 is confirmed, the remaining screening assessments begins.

Patients in Part 2 complete the ISM-SAF daily through screening to determine a baseline score, but inclusion in the study is not dependent upon a particular TSS. Accrual is stratified by TSS score (<28 and >28) and a minimum number of patients with TSS>28 are required.

Screening procedures for Parts 1, 2, and M include the following: BM biopsy (an archival sample obtained within the preceding 12 weeks or a fresh sample; for mMCAS patients a BM biopsy within the last 12 months is acceptable) and skin biopsy of lesional and non-lesional skin (in Part 1 or Part 2 patients with mastocytosis in skin). BM and skin biopsies are performed and sent to the Central Pathology Laboratory for confirmation of ISM or mMCAS diagnosis and quantification of MCs. Patients with mastocytosis in skin may opt to have skin photographs taken. Additional procedures include magnetic resonance imaging (MRI) or computed tomography scan of the brain, bone densitometry, serum tryptase, KIT D816V mutation testing, allele burden, routine laboratory testing, ECG, and physical examination. ISM or mMCAS diagnosis confirmation is required by the Central Laboratory.

Randomization in Part 1 and Part 2 occurs after patients are deemed eligible to participate following screening.

In Part 1 of the study, approximately 40 evaluable ISM patients with TSS≥28 are equally randomized (1:1:1:1 ratio) to 1 of 3 doses of Compound (I)+BSC or to placebo+BSC. The 3 dose levels (25 mg, 50 mg, or 100 mg) of Compound (I)+BSC and placebo+BSC are tested in parallel. Compound (I) is administered orally, once daily (QD) continuously. Patients are assessed weekly for the first 4 weeks for safety, laboratory monitoring, and quality of life (QoL) assessments. Pharmacokinetic sampling is performed in all patients. The pharmacokinetics data is unblinded after the completion of intensive PK collections (C1D1 and C1D15) of all patients. The ISM-SAF is completed daily. After completion of 12 weeks of treatment, BM and skin biopsy are repeated for MC quantification by the Central Pathology Laboratory and skin photographs (optional) may be taken in patients with baseline mastocytosis in skin. The RD is determined based on the efficacy, safety, and PK data at each dose level. The major efficacy criterion for selection of the RD is the dose of Compound (I) that produces the maximum reduction in TSS, as assessed using the ISM-SAF at Week 13 compared with Baseline. Other measures of efficacy (eg, change in serum tryptase) are also taken into consideration. Once Week 13 assessments are completed, patients continue on assigned therapy and dose until the RD is determined, at which time all Part 1 patients are unblinded and roll over to Part 3 where they receive Compound (I) in an open-label fashion at the RD.

In Part 2, of the study, up to 303 ISM patients are enrolled (at least 204 evaluable patients with TSS≥28 and up to 99 patients with TSS<28). Patients are randomly assigned to treatment at a 2:1 ratio to receive the RD of Compound (I)+BSC or matching placebo+BSC, respectively. Randomization are stratified based on TSS score (<28 and >28) and on centrally measured serum tryptase levels at screening (<20 ng/mL vs ≥20 ng/ml). In addition, enrollment of patients with <20 ng/mL serum tryptase are capped at approximately 20% for patients with TSS≥28 and approximately 20% for patients with TSS<28.

Compound (I) and placebo dosing are administered orally, QD, continuously. Patients are assessed every 4 weeks to Week 25 for safety, laboratory monitoring, and QoL assessments. Sparse PK sampling is performed in all patients. For patients with mastocytosis in skin who opt to do so, skin photographs are taken every 12 weeks. The ISM-SAF is completed daily.

After completion of 24 weeks of treatment, the ISM-SAF, BM, and skin biopsy are repeated for mast cell (MC) quantification by the Central Pathology Laboratory and skin photographs (optional) may be taken in patients with baseline mastocytosis in skin. Each patient completing the Week 25 assessments rolls over into the Part 3 long-term extension to receive the RD of Compound (I) QD in an open-label fashion. The Week 25 assessments are Baseline for Part 3. After all patients in Part 2 roll over into Part 3, all Part 2 treatment assignments are unblinded. At this point the primary endpoint of proportion of patients with a ≥30% reduction in TSS from Baseline to Week 25 and other efficacy endpoints is analyzed.

After all Part 1 patients have completed Part 1 and the RD has been determined or as each patient in Part 2 completes their Week 25 study assessments, patients roll over to Part 3. All patients receive open-label treatment with the RD of Compound (I).

In Part 3, Part 1 patients who received Compound (I) have study visits every 4 weeks until Week 25, then every 8 weeks until Week 49. After Week 49, patients have visits every 12 weeks for a total treatment duration of up to 5 years, inclusive of Part 1 or Part 2 as applicable. Part 1 patients who received placebo have weekly visits until Week 5.

In Part 3, patients rolling over from Part 2 have weekly visits until Week 5 and then follow the same schedule as Part 1 patients.

The ISM-SAF is completed daily for patients in Part 1 and Part 2, and QoL assessments are performed at study visits up to Part 3 Week 49 or End-of-Treatment (EOT), whichever occurs sooner. In patients who had or developed mastocytosis in their skin during Part 1 or Part 2, skin photographs (optional) may be taken at Part 3 Baseline (if not obtained within the prior 4 weeks in Part 1 or Part 2) and at other timepoints. Optional BM and skin biopsies are repeated for MC quantification by the Central Pathology Laboratory 1 year after the Part 1 Week 13 biopsies, or 1 year after the Part 2 Week 25 biopsies. For patients assigned to placebo in Part 1 or Part 2, the end of Part 1 or Part 2 study assessments, including BM and skin biopsies, serve as a baseline assessment for Part 3.

In Part M of the study, approximately 20 patients with mMCAS receive the RD of Compound (I) QD+BSC in an open-label single arm. The dose, formulation, and administration are the same as described for Part 2. This part does not include a placebo arm. After completion of 24 weeks of treatment, the BM biopsies are repeated for MC quantification by the Central Pathology. Patients continue with the long-term follow-up component of Part M.

1. A phosphate salt, a besylate salt, a sulfate salt, or a benzoate salt of Compound (I) represented by the following Formula: 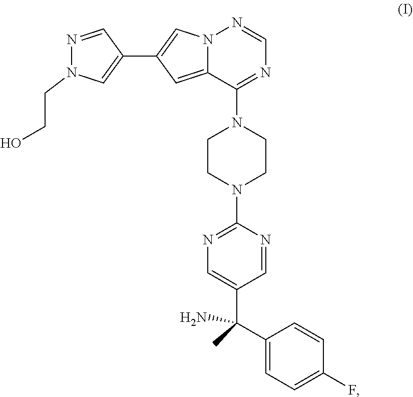 [SMILES: C[C@](N)(c1ccc(F)cc1)c1cnc(N2CCN(c3ncnn4cc(-c5cnn(CCO)c5)cc34)CC2)nc1]
wherein the molar ratio between Compound (I) and phosphoric acid is 1:1, the molar ratio between Compound (I) and benzene sulfonic acid is 2:1, the molar ratio between Compound (I) and sulfuric acid is 1:1, and the molar ratio between Compound (I) and benzoic acid is 1:1

2. The salt of claim 1, wherein the salt is unsolvated.

3. The salt of claim 2, wherein the salt is a phosphate salt.

4. (canceled)

5. The phosphate salt of claim 3, wherein the salt is a crystalline salt of Form A, characterized by an X-ray powder diffraction pattern which comprises peaks at 4.6°, 11.8°, 13.7°, 18.3°, and 19.8°±0.2 in 2θ.

6-10. (canceled)

11. The phosphate salt of claim 3, wherein the salt is a crystalline salt of Form G characterized by an X-ray powder diffraction pattern which comprises peaks at 4.1°, 4.4°, 7.4°, 15.6°, and 23.0°±0.2 in 2θ.

12-16. (canceled)

17. The phosphate salt of claim 3, wherein the salt is a crystalline salt of Form O, characterized by an X-ray powder diffraction pattern which comprises peaks at 4.2°, 15.2°, 21.6°, 21.9°, and 22.6°±0.2 in 2θ.

18-22. (canceled)

23. The salt of claim 2, wherein the salt is a besylate salt.

24-26. (canceled)

27. The besylate salt of claim 23, wherein the salt is a crystalline salt of Form 1-A, characterized by an X-ray powder diffraction pattern which comprises peaks at 5.7°, 7.1°, 9.7°, 15.4°, and 24.8°±0.2 in 2θ.

28-30. (canceled)

31. The besylate salt of claim 23, wherein the salt is a crystalline salt of Form 1-B, characterized by an X-ray powder diffraction pattern which comprises peaks at 5.5°, 10.4°, 14.0°, and 16.3°±0.2 in 2θ.

32-35. (canceled)

36. The salt of claim 2, wherein the salt is a sulfate salt.

37. (canceled)

38. The sulfate salt of claim 36, wherein the salt is a crystalline salt of Form 9-A, characterized by an X-ray powder diffraction pattern which comprises peaks at 9.0°, 10.4°, 13.6°, 18.1°, and 21.0°±0.2 in 2θ.

39-44. (canceled)

45. The salt of claim 2, wherein the salt is a benzoate salt.

46. (canceled)

47. The benzoate salt of claim 45, wherein the salt is a crystalline salt of Form 2-A, characterized by an X-ray powder diffraction pattern which comprises peaks at 4.1°, 10.6°, 16.8°, 18.1°, and 22.7°±0.2 in 2θ.

48-53. (canceled)

54. The benzoate salt of claim 45, wherein the salt is a crystalline salt of Form 2-B, characterized by an X-ray powder diffraction pattern which comprises peaks at 5.8°, 9.7°, 15.4°, and 17.8°±0.2 in 2θ.

55-57. (canceled)

58. An amorphous form of Compound (I) represented by the following Formula:

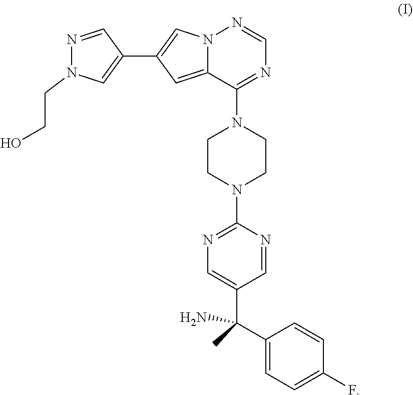 [SMILES: C[C@](N)(c1ccc(F)cc1)c1cnc(N2CCN(c3ncnn4cc(-c5cnn(CCO)c5)cc34)CC2)nc1]

59. (canceled)

60. A pharmaceutical composition comprising the salt of Compound (I) of claim 1, and a pharmaceutically acceptable carrier or diluent.

61. A method of treating a disease or condition in a patient in need thereof, wherein the method comprises administering to the patient the salt of Compound (I) of claim 1, wherein the disease or condition is chosen from systemic mastocytosis, gastrointestinal stromal tumors, acute myeloid leukemia, melanoma, seminoma, mediastinal B-cell lymphoma, Ewing's sarcoma, diffuse large B cell lymphoma, dysgerminoma, myelodysplastic syndrome, nasal NK/T-cell lymphoma, chronic myelomonocytic, and leukemia.

62. A method of treating indolent systemic mastocytosis (ISM) or monoclonal mast cell activation syndrome (mMCAS) comprising orally administering to a patient in need thereof an amount of 15 mg to 200 mg of Compound (I) 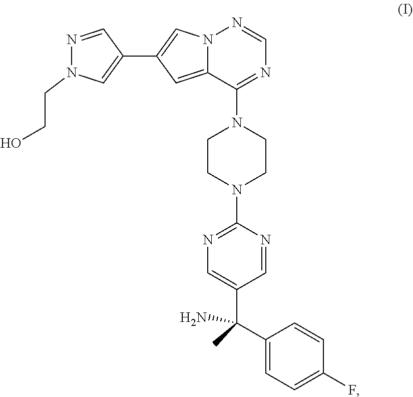 [SMILES: C[C@](N)(c1ccc(F)cc1)c1cnc(N2CCN(c3ncnn4cc(-c5cnn(CCO)c5)cc34)CC2)nc1]
or a pharmaceutically acceptable salt thereof in an amount equivalent to 15 mg to 200 mg of Compound (I), once a day.

63. A process for the preparation of crystalline phosphate salt Form A of Compound (I) of claim 5: 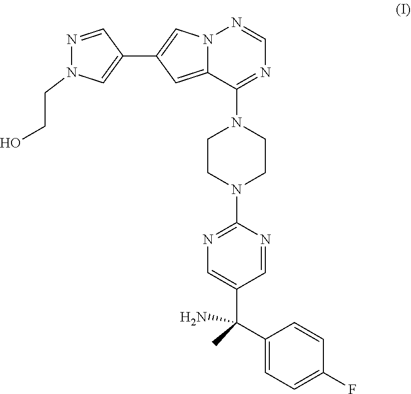 [SMILES: C[C@](N)(c1ccc(F)cc1)c1cnc(N2CCN(c3ncnn4cc(-c5cnn(CCO)c5)cc34)CC2)nc1]
comprising forming a phosphate salt of Compound (I) with phosphoric acid in an organic solvent mixture comprising 2-MeTHF/acetone/water.

64-65. (canceled)

66. The process of claim 63, further comprising crystallizing phosphate salt Form A Compound (I) by adding acetone.

67. (canceled)
